SEQUENCE LISTING (206 sequences)

SEQ ID NO: 1 - protein, 129 aa, Artificial Sequence
  AceL-TerL-11
CVYGDTMVETEDGKIKIEDLYKRLAMFRTNTNNIKILSPNGFSNFNGIQKVERNLYQHIIFDDDTEIKTSINHPFGKDKILARDVKVGDYLNSKKVLYNELVNENIFLYDPINVEKESLYITNGVVSHN

SEQ ID NO: 2 - protein, 24 aa, Artificial Sequence
  IntN WT AceL-TerL-3
CVDGNTIVETEDGKIKIEDLYKKL

SEQ ID NO: 3 - protein, 24 aa, Artificial Sequence
  IntN WT AceL-Terl4
CVDGNTIVETEDGKIKIEDLYKKM

SEQ ID NO: 4 - protein, 24 aa, Artificial Sequence
  IntN WT AceL-Terl5
CVDGNTIVETEDGKIKIEDLYKKL

SEQ ID NO: 5 - protein, 25 aa, Artificial Sequence
  AceL-TerL-11 IntN
CVYGDTMVETEDGKIKIEDLYKRLA

SEQ ID NO: 6 - protein, 104 aa, Artificial Sequence
  AceL-TerL-11 IntC
MFRTNTNNIKILSPNGFSNFNGIQKVERNLYQHIIFDDDTEIKTSINHPFGKDKILARDVKVGDYLNSKKVLYNELVNENIFLYDPINVEKESLYITNGVVSHN

SEQ ID NO: 7 - protein, 660 aa, Artificial Sequence
  MBP-AceL-TerLcis-FKBP-His6, pIT063
MEIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFEFECVYGDTMVETEDGKIKIEDLYKRLAMGMFRTNTNNIKILSPNGFSNFNGIQKVERNLYQHIIFDDDTEIKTSINHPFGKDKILARDVKVGDYLNSKKVLYNELVNENIFLYDPINVEKESLYITNGVVSHNCEFLSRNNGNGNGTRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSYGSRSHHHHHH

SEQ ID NO: 8 - protein, 665 aa, Artificial Sequence
  MBP-AceL-TerLcis-FKBP-His6 pIT064
MEIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFEFECVYGDTMVETEDGKIKIEDLYKRLAMGSGGSGMFRTNTNNIKILSPNGFSNFNGIQKVERNLYQHIIFDDDTEIKTSINHPFGKDKILARDVKVGDYLNSKKVLYNELVNENIFLYDPINVEKESLYITNGVVSHNCEFLSRNNGNGNGTRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSYGSRSHHHHHH

SEQ ID NO: 9 - protein, 660 aa, Artificial Sequence
  pIT065inaktiv(N129A,C+1A
MEIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFEFECVYGDTMVETEDGKIKIEDLYKRLAMGMFRTNTNNIKILSPNGFSNFNGIQKVERNLYQHIIFDDDTEIKTSINHPFGKDKILARDVKVGDYLNSKKVLYNELVNENIFLYDPINVEKESLYITNGVVSHAAEFLSRNNGNGNGTRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSYGSRSHHHHHH

SEQ ID NO: 10 - protein, 665 aa, Artificial Sequence
  pIT066inaktiv(N129A,C+1A)
MEIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFEFECVYGDTMVETEDGKIKIEDLYKRLAMGSGGSGMFRTNTNNIKILSPNGFSNFNGIQKVERNLYQHIIFDDDTEIKTSINHPFGKDKILARDVKVGDYLNSKKVLYNELVNENIFLYDPINVEKESLYITNGVVSHAAEFLSRNNGNGNGTRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSYGSRSHHHHHH

SEQ ID NO: 11 - protein, 25 aa, Artificial Sequence
  M1N
CVYGDTMVETEDGKIKIEDLYKRLT

SEQ ID NO: 12 - protein, 25 aa, Artificial Sequence
  M3N
CVSGDTMVETEDGKIKIEDLYKRLA

SEQ ID NO: 13 - protein, 104 aa, Artificial Sequence
  M1C
MFRTNTNNIKILSPNGFSNFDGIQKVERNQYQHIIFDDDTEIKTSINHPFGKDKILARDVKVGDYLNSKKVLYNELVNENIFLYDPINVEKESLYITNGVVSHN

SEQ ID NO: 14 - protein, 104 aa, Artificial Sequence
  M2C
MFRTNTNNIKILGPNGFSNFIGIQKVERDQYQHIIFDDDTEIKTSINHPFGKDKILARDVKVGDYLNSKKVLYNELVNENIFLYDPINVEKESLYITNGVVSHN

SEQ ID NO: 15 - protein, 104 aa, Artificial Sequence
  M3C
MFRTNTNNIKILSPNGFSNFNGIQKVERNLYQHIIFDDDTEIKTSINHPFGKDKILARDVKVGDYLNGKKVLYNELVNENIFLYDPINVEKESLYITNGVVSHN

SEQ ID NO: 16 - protein, 104 aa, Artificial Sequence
  M4C
MFRTNTNNIKILSPNGFSNFIGIQKVERNQYQHIIFDDDTEIKTSINHPFGKDKILARDVKVGDYLNSKKVLYNELVNENIFLYDPINVEKESLYITNGVVSHN

SEQ ID NO: 17 - protein, 104 aa, Artificial Sequence
  M5C
MFRTNTNNIKILSPNGFSNFDGIQKVERNLYQHIIFDDDTEIKTSINHPFGKDKILARDVKVGDYLNSKKVLYNELVNENIFLYDPINVEKESLYITNGVVSHN

SEQ ID NO: 18 - protein, 104 aa, Artificial Sequence
  M6C
MFRTNTNNIKILSPNGFSNFNGIQKVERNQYQHIIFDDDTEIKTSINHPFGKDKILARDVKVGDYLNSKKVLYNELVNENIFLYDPINVEKESLYITNGVVSHN

SEQ ID NO: 19 - DNA, 312 bp, Artificial Sequence
  DNA M1C
atgtttcgtaccaacaccaacaacattaaaattctgagcccgaacggctttagtaactttgacggcattcagaaagtggaacgtaaccagtatcagcatattatttttgatgatgataccgaaattaaaaccagcattaaccatccgtttggcaaagataaaattctggcgcgtgatgtgaaagtgggcgattatctgaacagcaaaaaagtgctgtataacgaactggtgaacgaaaacatttttctgtatgatccgattaacgtggaaaaagaaagcctgtatattaccaacggcgtggtgagccataac

SEQ ID NO: 20 - DNA, 312 bp, Artificial Sequence
  DNA  M2C
atgtttcgtaccaacaccaacaacattaaaattctgggcccgaacggctttagcaactttatcggcattcagaaagtggaacgtgaccagtatcagcatattatttttgatgatgataccgaaattaaaaccagcattaaccatccgtttggcaaagataaaattctggcgcgtgatgtgaaagtgggcgattatctgaacagcaaaaaagtgctgtataacgaactggtgaacgaaaacatttttctgtatgatccgattaacgtggaaaaagaaagcctgtatattaccaacggcgtggtgagccataac

SEQ ID NO: 21 - DNA, 312 bp, Artificial Sequence
  DNA M3C
atgtttcgtaccaacaccaacaacattaaaattctgagcccgaacggctttagcaactttaacggcattcagaaagtggaacgtaacctgtatcagcatattatttttgatgatgataccgaaattaaaaccagcattaaccatccgtttggcaaagataaaattctggcgcgtgatgtgaaagtgggcgattatctgaacggcaaaaaagtgctgtataacgaactggtgaacgaaaacatttttctgtatgatccgattaacgtggaaaaagaaagcctgtatattaccaacggcgtggtgagccataac

SEQ ID NO: 22 - DNA, 312 bp, Artificial Sequence
  DNA  M4C
atgtttcgtaccaacaccaacaacattaaaattctgagcccgaacggctttagcaactttatcggcattcagaaagtggaacgtaaccagtatcagcatattatttttgatgatgataccgaaattaaaaccagcattaaccatccgtttggcaaagataaaattctggcgcgtgatgtgaaagtgggcgattatctgaacagcaaaaaagtgctgtataacgaactggtgaacgaaaacatttttctgtatgatccgattaacgtggaaaaggaaagcctgtatattaccaacggcgtggtgagccataac

SEQ ID NO: 23 - DNA, 312 bp, Artificial Sequence
  DNA  M5C
atgtttcgtaccaacaccaacaacattaaaattctgagcccgaacggctttagcaactttgacggcattcagaaagtggaacgtaacctgtatcagcatattatttttgatgatgataccgaaattaaaaccagcattaaccatccgtttggcaaagataaaattctggcgcgtgatgtgaaagtgggcgattatctgaacagcaaaaaagtgctgtataacgaactggtgaacgaaaacatttttctgtatgatccgattaacgtggaaaaagaaagcctgtatattaccaacggcgtggtgagccataac

SEQ ID NO: 24 - protein, 5 aa, Artificial Sequence
  IntN WTAceL-TerL-11 flanking sequence
QTEFE

SEQ ID NO: 25 - protein, 5 aa, Artificial Sequence
  IntC WTAceL-TerL-11 flanking sequence
CEFLG

SEQ ID NO: 26 - protein, 152 aa, Artificial Sequence
  GS033_TerA-6intein
SISQESYINIEVNGKVETIKIGDLYKKLSFNERKFNEMKLPESVVKNNINLKIETPYGFENFYGVNKIKKDKYIHLEFTNGEKLKCSLDHPLSTIDGIVKAKDLDKYTEVYTKFGGCFLKKSKVINESIELYDIVNSGLKHLYYSNNIISHN

SEQ ID NO: 27 - protein, 115 aa, Artificial Sequence
  Wild type IntC of GS033_TerA-6
MKLPESVVKNNINLKIETPYGFENFYGVNKIKKDKYIHLEFTNGEKLKCSLDHPLSTIDGIVKAKDLDKYTEVYTKFGGCFLKKSKVINESIELYDIVNSGLKHLYYSNNIISHN

SEQ ID NO: 28 - protein, 37 aa, Artificial Sequence
  Wild type IntN of GS033_TerA-6
SISQESYINIEVNGKVETIKIGDLYKKLSFNERKFNE

SEQ ID NO: 29 - protein, 37 aa, Artificial Sequence
  IntN of GS033_TerA-6
CISQESYINIEVNGKVETIKIGDLYKKLSFNERKFNE

SEQ ID NO: 30 - protein, 28 aa, Artificial Sequence
  IntN of GS033_TerA-6 (minus 9aa)
SISQESYINIEVNGKVETIKIGDLYKKL

SEQ ID NO: 31 - protein, 28 aa, Artificial Sequence
  IntN of GS033_TerA-6 (minus 9aa synthetic)
SISQESYINIEVNGKVETIKIGDLYKKL

SEQ ID NO: 32 - protein, 34 aa, Artificial Sequence
  IntN of GS033_TerA-6 (minus 3aa)
SISQESYINIEVNGKVETIKIGDLYKKLSFNERK

SEQ ID NO: 33 - protein, 25 aa, Artificial Sequence
  IntNAceL-TerL-11 with C1S substitution
SVSGDTMVETEDGKIKIEDLYKRLA

SEQ ID NO: 34 - protein, 5 aa, Artificial Sequence
  IntN GS033_TerA-6 flanking sequence
KVEFE

SEQ ID NO: 35 - protein, 5 aa, Artificial Sequence
  Intc GS033_TerA-6 flanking sequence
CEFLG

SEQ ID NO: 36 - DNA, 128 bp, Artificial Sequence
  DNA  IntN  GS033_TerA-6
aggttgagtttgagtcaatttctcaagaatcttacataaatatcgaagttaatggtaaggtcgaaacaattaaaattggcgatttatataaaaaactttcatttaacgaaagaaaatttaatgagtga

SEQ ID NO: 37 - DNA, 360 bp, Artificial Sequence
  DNA  IntC  GS033_TerA-6
atgaaattaccagaatctgtagtaaaaaacaatatcaacttaaaaatagaaactccatatggatttgagaatttttatggagtaaataaaataaagaaggataagtatatacatttagaatttaccaatggtgaaaaactaaagtgctctttagatcatccattatcaacaattgatggaattgtaaaagcaaaagatttagacaaatatacagaagtatatacaaaatttggtggatgctttctaaaaaaatcaaaagttattaatgaatcaatagaattatatgatattgtaaactcgggactaaagcatttatattattcaaataatataatatctcacaactgcgaattcttaggg

SEQ ID NO: 38 - protein, 36 aa, Artificial Sequence
  IntN TerA-1_CP21-BP
CCLENTRVQVRNKYTNKIETLTIKELYARLQELKKS

SEQ ID NO: 39 - protein, 104 aa, Artificial Sequence
  IntC TerA-1_CP21-BP
MSEIQDINPYEILTPQGFKPFVDIIKSIQTTGITITLEDSREISVTLDHKFKHLNDYKEAKYFKVGDKLQCSKIIKIENIEGEFYEPLEVQDHEYIANDFINHN

SEQ ID NO: 40 - protein, 5 aa, Artificial Sequence
  IntN TerA-1_CP21-BP flanking sequence
FRGFS

SEQ ID NO: 41 - protein, 5 aa, Artificial Sequence
  IntC TerA-1_CP21-BP flanking sequence
CNIIV

SEQ ID NO: 42 - DNA, 126 bp, Artificial Sequence
  DNA IntN TerA-1_CP21-BP
tttaggggattcagttgctgtctcgaaaatactcgagtgcaggtaagaaataaatatactaataaaatagaaacgcttaccataaaggaattgtatgctaggttacaagaactcaaaaaatcttaa

SEQ ID NO: 43 - DNA, 327 bp, Artificial Sequence
  DNA IntC TerA-1_CP21-BP
gtgagtgaaatccaagatataaatccatatgaaatattaacaccacaaggatttaaaccttttgttgatatcattaaatcaattcaaacaactggcataacaataactttagaggattcaagagagatatcagttacattagatcacaaatttaaacacttaaatgattataaagaagccaaatattttaaagtaggtgataaattacagtgttcaaaaattattaaaattgaaaatattgaaggtgaattttatgaacctttagaagttcaagatcacgagtatatagccaacgactttataaatcataattgtaatataatcgtt

SEQ ID NO: 44 - protein, 31 aa, Artificial Sequence
  IntN  CP81-BP_TerA
CVAGDTKITVRNKKTGVIEDITMEELYNRIG

SEQ ID NO: 45 - protein, 100 aa, Artificial Sequence
  IntC CP81-BP_TerA
MYEVLTPNGFSDFDDISREKKDVYKVITEDDFIKVTKGHKFETPNGFKQLKHLKINDLIKYKNKFSKIVLIDYVGVEYVYDLINVHKNNEYYTNNFVSHN

SEQ ID NO: 46 - protein, 5 aa, Artificial Sequence
  IntN CP81-BP_TerA flanking sequence
IFIDE

SEQ ID NO: 47 - protein, 5 aa, Artificial Sequence
  Intc CP81-BP_TerA flanking sequence
CAFID

SEQ ID NO: 48 - DNA, 111 bp, Artificial Sequence
  DNA  IntN CP81-BP_TerA
atttttattgatgaatgtgtagctggtgacacaaaaattacagttagaaataagaaaacaggtgtcattgaagatataacaatggaagagttatataacagaataggataa

SEQ ID NO: 49 - DNA, 315 bp, Artificial Sequence
  DNA IntC CP81-BP_TerA
atgtatgaagtactaacaccaaatggatttagtgattttgatgatatatcaagagaaaaaaaagatgtatataaagtaataacagaagatgattttataaaagtaacaaaaggtcataaatttgaaacacctaatggttttaaacaattaaaacatcttaaaattaatgatttaataaaatataaaaataaattttcaaaaattgttttaatagattatgttggagtagaatatgtatatgatttaattaatgtacataaaaataacgagtattatacaaataattttgtttcacacaattgtgcgtttatagat

SEQ ID NO: 50 - protein, 33 aa, Artificial Sequence
  IntN AceL-1_ClpC-1
CFSKKTSIKLRNKKTGDLEEIDISDLIYELHIS

SEQ ID NO: 51 - protein, 125 aa, Artificial Sequence
  IntC AceL-1_ClpC-1
MIKLYNKKQNKKFTKSYDLGDYQILTDSGYIGLVSLHETIPYEVWKLKLSNGYELECADDHIIFDNEMNEIFVKNLELGDRVKVDDGYAVVIELVNTGLLESMYDFELVEDSNRRYYTNGILSHN

SEQ ID NO: 52 - protein, 5 aa, Artificial Sequence
  IntN AceL-1_ClpC-1 flanking sequence
SGVGK

SEQ ID NO: 53 - protein, 5 aa, Artificial Sequence
  IntC AceL-1_ClpC-1flanking sequence
TELAK

SEQ ID NO: 54 - DNA, 117 bp, Artificial Sequence
  DNA  IntN AceL-1_ClpC-1
agcggggttggtaaatgtttttctaaaaaaacatcaataaaattaaggaataaaaaaactggtgatttagaagaaattgatatttctgatctaatatatgaactacacattagctaa

SEQ ID NO: 55 - DNA, 390 bp, Artificial Sequence
  DNA IntC AceL-1_ClpC-1
atgataaaattatataataaaaaacaaaataaaaaattcaccaaatcttatgatttgggtgattaccaaatactaactgatagtggatatattggtttggtctcattacatgagacaataccatatgaagtttggaaattgaaattatctaatggatatgaattagagtgtgctgatgatcatattatttttgataatgaaatgaatgagatatttgtaaagaatctagaattaggagacagagtaaaagtagatgatggatatgctgttgttatagaattagtaaatactggtctattagaaagtatgtatgattttgagttagtagaagattcaaatagaaggtattatacaaatggtattttatcacacaacacagaactggctaaa

SEQ ID NO: 56 - protein, 30 aa, Artificial Sequence
  IntN AceL-1_ClpC-2
CVSPNTKIKIRNSSTGEISEVTIAEFNKMI

SEQ ID NO: 57 - protein, 115 aa, Artificial Sequence
  IntC AceL-1_ClpC-2
MKKIVKSVSVEGFEVLSDNGWVPIKNVHTTVPYELYNLRTANGLRLECADNHIVFTSKLKEVYVKDLNVDDKIMTEDGVSLVSSIEKTKAKVTMYDLEVDSEDHRYYTDGILSHN

SEQ ID NO: 58 - protein, 5 aa, Artificial Sequence
  IntN AceL-1_ClpC-2 flanking sequence
AGVGK

SEQ ID NO: 59 - protein, 5 aa, Artificial Sequence
  IntCAceL-1_ClpC-2 flanking sequence
TSLIE

SEQ ID NO: 60 - DNA, 108 bp, Artificial Sequence
  DNA I IntN AceL-1_ClpC-2
gcaggagtaggtaaatgcgttagtcctaatacgaagattaagattaggaacagtagcactggagaaatttcagaagttacgatagcggaattcaataagatgatttaa

SEQ ID NO: 61 - DNA, 360 bp, Artificial Sequence
  DNA IntC AceL-1_ClpC-2
atgaaaaaaattgttaagagtgtaagtgtagaaggatttgaggtactctctgataatggatgggtaccaattaaaaatgtacataccactgtaccctacgaactctataacctccgtacagccaacggtttgcggttagaatgtgcagacaatcatatcgtgtttacttctaagctaaaagaggtatatgttaaagacttaaatgttgacgataagattatgactgaggatggagtatctttagtatcatcaattgaaaagactaaagctaaagtaacgatgtatgatcttgaagtagatagtgaagatcatcgttactatactgacggtattctttcacataacacttctctaatagaa

SEQ ID NO: 62 - protein, 34 aa, Artificial Sequence
  IntN AceL-1_RadAl-1
CVHPNTLVKIKIDSTGEERTITVKDLHELIKSVK

SEQ ID NO: 63 - protein, 115 aa, Artificial Sequence
  IntC AceL-1_RadAl-1
MKRKFIESISADNISIMTDTGWEKVKGSHVTIEYKVFNLVTDRLSLQCADDHIVFKEDFSEVFVKDLEVGDLIQTVNGLESVTEVYETDDLVNMHDLEIDSKNHRYYTDGILSHN

SEQ ID NO: 64 - protein, 5 aa, Artificial Sequence
  IntN AceL-1_RadAl-1 flanking sequence
PGVGK

SEQ ID NO: 65 - protein, 5 aa, Artificial Sequence
  IntC AceL-1_RadAl-1 flanking sequence
TTLLL

SEQ ID NO: 66 - DNA, 120 bp, Artificial Sequence
  DNA IntN AceL-1_RadAl-1
ccaggagttggtaaatgcgtccatccaaatacattggtaaaaatcaaaattgattctactggtgaggagcgtactattacagtcaaagacctccacgaactaattaaatctgtaaaatga

SEQ ID NO: 67 - DNA, 360 bp, Artificial Sequence
  DNA IntC AceL-1_RadAl-1
atgaaacgtaaatttatagaaagtatttctgcagacaatatcagcatcatgacagatactggttgggaaaaagttaaaggtagtcacgttacaattgagtataaagtattcaaccttgtcactgacaggttatcactacaatgtgcagatgatcatatcgtttttaaagaggacttctcagaggtctttgtaaaggaccttgaggttggtgatttaatacaaacagtaaacggtttagaatcagttactgaagtatatgaaacagacgacttggtaaatatgcacgatttagaaattgattctaaaaaccataggtattatactgatggaattctttcacataatactacattattattg

SEQ ID NO: 68 - protein, 34 aa, Artificial Sequence
  IntN AceL-1_TerL-10
CVSGDTKVTLKDNDTGKIINVNIEEMVSVSSLDV

SEQ ID NO: 69 - protein, 105 aa, Artificial Sequence
  IntC AceL-1_TerL-10
MEVGKMSKSYKVLSPSGFVDFAGIQKITRSKYRHFIFDDGTEIKCSLNHRFGEEEIVASTLHHGTELQGKKILYAEDVEDDIDLYDLLNVANGNLYYTNGLVSHN

SEQ ID NO: 70 - protein, 5 aa, Artificial Sequence
  IntN AceL-1_TerL-10 flanking sequence
NTEFE

SEQ ID NO: 71 - protein, 5 aa, Artificial Sequence
  IntC AceL-1_TerL-10 flanking sequence
CEFLG

SEQ ID NO: 72 - DNA, 120 bp, Artificial Sequence
  DNA IntN AceL-1_TerL-10
aacacggagtttgagtgtgtttctggtgatacaaaggttactctcaaagacaatgatacaggaaagattattaatgtaaatattgaagaaatggtgagtgtgagttctttggatgtataa

SEQ ID NO: 73 - DNA, 330 bp, Artificial Sequence
  DNA IntC AceL-1_TerL-10
atggaagttggaaagatgtctaaaagttataaagtgttatcaccatcagggtttgtggattttgctggtattcaaaaaataacacgcagcaaatatcgacattttatttttgatgatggcacagaaatcaaatgttcgttaaatcatagatttggtgaagaggaaatagtagcctcaacactccatcacggcacagagcttcagggtaaaaaaatactgtatgcagaagatgttgaggatgatattgatttatatgatttgttaaatgttgccaatggaaatctttactacaccaacggattagtatcacacaattgtgagttccttggc

SEQ ID NO: 74 - protein, 55 aa, Artificial Sequence
  IntN AceL-1_TerL-2
CFFFNTIISVETNNQQYETRIGILYYSMVSKERNLTILEKIKIKLYDLLFILEKH

SEQ ID NO: 75 - protein, 150 aa, Artificial Sequence
  IntC AceL-1_TerL-2
MLRIFKRCLIYLIKKMIEFIELYEYKKISLDECDINKKILNSISLMDLKVETDTGYETSSNIHITQPFKHYNIETVDGYEIICADNHILFDEEFNEVFTKDLKIGDLIKTKNGNSVIKSIYIDTHKSSMFDLTIDHPNHRFYTNGILSHN

SEQ ID NO: 76 - protein, 5 aa, Artificial Sequence
  IntNAceL-1_TerL-2 flanking sequence
RQVGK

SEQ ID NO: 77 - protein, 5 aa, Artificial Sequence
  IntCAceL-1_TerL-2flanking sequence
TISSS

SEQ ID NO: 78 - DNA, 183 bp, Artificial Sequence
  DNA  IntN AceL-1_TerL-2
aggcaagttggaaaatgtttttttttcaatacaattatatccgttgaaaccaataatcagcaatatgaaactagaataggaattctttattattcaatggtttcaaaggaaagaaatttaactattttagagaaaattaaaataaaattatatgatttattattcattttagaaaaacattaa

SEQ ID NO: 79 - DNA, 465 bp, Artificial Sequence
  DNA  IntC AceL-1_TerL-2
atgcttagaatttttaaaaggtgtttaatttatttaattaaaaaaatgattgaatttattgaattatatgaatataaaaaaatctcattagatgagtgtgatataaataaaaaaatattaaactcaatatctcttatggatttaaaggtagagactgatacaggatatgaaacatcatctaatatacacataacacaaccatttaaacactataatattgaaacggttgatggttatgaaataatatgtgctgataatcatatattatttgatgaggaattcaatgaagtatttacaaaggatttaaaaataggagatttaattaaaacaaaaaatggcaacagtgttatcaagagtatttatatagacacacataagtcatccatgtttgacctaacaatagaccatccaaaccacaggttctatacaaatggtatactttcacataatacgatatcatcttct

SEQ ID NO: 80 - protein, 24 aa, Artificial Sequence
  IntN AceL-1_TerL-3
CVDGNTIVETEDGKIKIEDLYKKL

SEQ ID NO: 81 - protein, 104 aa, Artificial Sequence
  IntC AceL-1_TerL-3
MFITNTDNIKILSPSGFSNFNGIQKVERNLYQHIIFDDESEIKTSINHPFGKNKILARNVKVGDYLSSKKVLYNELVNEKIFLYDPINVEKENLYITNGVVSHN

SEQ ID NO: 82 - protein, 5 aa, Artificial Sequence
  IntN AceL-1_TerL-3 flanking sequence
QQEFE

SEQ ID NO: 83 - protein, 5 aa, Artificial Sequence
  IntC AceL-1_TerL-3 flanking sequence
CEFLG

SEQ ID NO: 84 - DNA, 90 bp, Artificial Sequence
  DNA  IntN  AceL-1_TerL-3
caacaagagtttgaatgtgttgacggtaatacgatagtcgaaacggaagatggtaaaataaaaatagaagatttatataaaaaattgtga

SEQ ID NO: 85 - DNA, 327 bp, Artificial Sequence
  DNA IntCAceL-1_TerL-3
atgtttataactaatacagataatataaaaatattaagtccaagtggattttctaattttaatggtattcaaaaggttgaaagaaacctttatcaacacattatctttgatgatgaatctgaaataaaaacttctattaaccacccttttggtaaaaataaaatattagcaagaaatgtaaaagtaggagattatttaagtagtaaaaaagtattatataatgagttggttaatgaaaaaatatttttatatgaccctataaatgtagaaaaagaaaacttatatattactaacggtgttgtttctcataattgtgagtttttaggt

SEQ ID NO: 86 - protein, 24 aa, Artificial Sequence
  IntN AceL-1_TerL-4
CVDGNTIVETEDGKIKIEDLYKKM

SEQ ID NO: 87 - protein, 104 aa, Artificial Sequence
  IntC AceL-1_TerL-4
MFRTNTDNIKILSPSGFSIFNGIQKVERDLYQHIIFDDKSEIKTSINHPFGKDKILARNIKVGDYLNSKKVLYNELVAEKITLYDPINVEKENLYITNGVISHN

SEQ ID NO: 88 - protein, 5 aa, Artificial Sequence
  IntN AceL-1_TerL4 flanking sequence
QQEFE

SEQ ID NO: 89 - protein, 5 aa, Artificial Sequence
  IntC AceL-1_TerL-4 flanking sequence
CEFLG

SEQ ID NO: 90 - DNA, 90 bp, Artificial Sequence
  DNA  IntN  AceL-1_TerL-4
caacaagagtttgagtgtgtggatggaaatacgatagtcgaaacggaagatggcaaaataaaaatagaagatttatataaaaaaatgtga

SEQ ID NO: 91 - DNA, 327 bp, Artificial Sequence
  DNA IntC AceL-1_TerL-4
atgtttagaacaaatacagataatataaaaattttaagtccaagtgggttttctatttttaatggcattcaaaaggttgaaagagacctctatcaacatattatctttgatgataaatctgaaataaagacttctatcaaccacccctttggtaaagataaaatattagcgagaaatataaaggttggtgattatttaaatagtaagaaagttttatataatgagttggtcgccgaaaagattactttatatgatcctataaatgtagaaaaagaaaatttatatatcactaacggtgttatttctcataattgtgagtttttaggt

SEQ ID NO: 92 - protein, 24 aa, Artificial Sequence
  IntN AceL-1_TerL-5
CVDGNTIVETEDGKIKIEDLYKKL

SEQ ID NO: 93 - protein, 104 aa, Artificial Sequence
  IntC AceL-1_TerL-5
MFRTNTDNIKILSPSGFSNFNGIQKVERDLYQHIIFDDKSEIKTSINHPFGKDKILARNIKVGDYLNSKKVLYNELVNEKITLYDPINVEKENLYITNGVISHN

SEQ ID NO: 94 - protein, 5 aa, Artificial Sequence
  IntN AceL-1_TerL5 flanking sequence
QQEFE

SEQ ID NO: 95 - protein, 5 aa, Artificial Sequence
  IntC AceL-1_TerL-5 flanking sequence
CEFLG

SEQ ID NO: 96 - DNA, 90 bp, Artificial Sequence
  DNA  IntN  AceL-1_TerL-5
caacaagagtttgaatgtgttgacggtaatacgatagttgaaacggaagatggtaaaataaaaatagaagatttatataaaaaattatag

SEQ ID NO: 97 - DNA, 327 bp, Artificial Sequence
  DNA  IntC AceL-1_TerL-5
atgtttagaaccaatacagataatataaaaatattaagtccaagtggattttctaattttaacggcattcaaaaggttgaaagagacctctatcaacatattatctttgatgataagtctgaaataaaaacttctattaaccacccttttggtaaagataaaatattagcgagaaatataaaagtaggagattatttaaatagtaagaaggttttatataatgagttggttaatgaaaaaattactttatatgaccctataaatgtagaaaaagaaaacttatatattactaacggtgttatttctcataattgtgagtttttaggt

SEQ ID NO: 98 - protein, 27 aa, Artificial Sequence
  IntN AceL-1_UvsW-3-1
CRTYDSTMDIDVGNSDFAEYLLNNSKK

SEQ ID NO: 99 - protein, 135 aa, Artificial Sequence
  IntC AceL-1_UvsW-3-1
MKFNIPIGELAESIAKYKGVLLNDNCEINIKDLDCKVNTPSGTATINIIIKKEKLEGIKLLLANGVEIKCANKHILRYNNADVFADSLAIGDSVETINGNVKVSSINNIDDTTFYDIGIDAPYLYYDADGVLHHN

SEQ ID NO: 100 - protein, 5 aa, Artificial Sequence
  IntN AceL-1_UvsW-3-1 flanking sequence
TGAGK

SEQ ID NO: 101 - protein, 5 aa, Artificial Sequence
  IntC AceL-1_UvsW-3-1 flanking sequence
TITTA

SEQ ID NO: 102 - DNA, 99 bp, Artificial Sequence
  DNA  IntN AceL-1_UvsW-3-1
actggtgcaggcaaatgtcgaacttatgattctacaatggatatagatgtaggtaattctgattttgctgaatatttgctaaataatagtaagaaatag

SEQ ID NO: 103 - DNA, 420 bp, Artificial Sequence
  DNA IntC AceL-1_UvsW-3-1
atgaaatttaacataccaataggggaactagcagagtcgatcgcgaagtacaaaggagtactattaaacgataactgcgaaattaatattaaagatcttgattgtaaagttaatacaccatcaggaactgctactattaatattataattaaaaaagaaaagttagaaggcataaaactattacttgcaaatggtgtagaaataaagtgtgctaataagcatatattaagatataataatgcagacgtatttgcagattcattagcaattggcgactcggtagaaactattaacgggaatgttaaggttagtagtattaacaatattgacgatactacattttacgatatcggaatagatgcaccgtacttatattatgatgcagacggagtattacatcataatacaattaccacagca

SEQ ID NO: 104 - protein, 37 aa, Artificial Sequence
  IntN AceL-1_gp41-1
CFFSDGEINTRNISNKEIKSIKIGKIFTNISKGHTNI

SEQ ID NO: 105 - protein, 148 aa, Artificial Sequence
  IntC AceL-1_gp41-1
MLDNYEIIEADSLLEGKYDRPLYDKFIEAYEVDNLEVDTPNGWIKIEGIGKTIEFYEWEIQTSGGKHLICADKHLLYRCDNMNFYNKKCDITEIYCQDLNIGDFIMTKDGPEMLMDIYKNGNKSNMYDLQLSEGSNKQYYTNDILSHN

SEQ ID NO: 106 - protein, 5 aa, Artificial Sequence
  IntN AceL-1_gp41-1flanking sequence
SLWMQ

SEQ ID NO: 107 - protein, 5 aa, Artificial Sequence
  IntC AceL-1_gp41-1flanking sequence
TNGGK

SEQ ID NO: 108 - DNA, 129 bp, Artificial Sequence
  DNA  IntN AceL-1_gp41-1
acaaacggaggaaaatgtttttttagtgatggtgagataaatactaggaatataagcaataaggaaataaaatcaattaaaataggtaaaatttttaccaacattagcaagggacatactaacatttaa

SEQ ID NO: 109 - DNA, 459 bp, Artificial Sequence
  DNA IntC AceL-1_gp41-1
atgttagataattatgaaataatagaagcagattctctattagaaggaaaatatgatagaccactatatgataaatttattgaagcttatgaagtagacaacttagaggttgatacaccaaatggttggataaagatagaaggaattggtaaaactattgaattttatgaatgggaaatacaaacatctggtggaaaacatctaatatgtgcagataaacatctattatataggtgtgataatatgaatttttataataaaaaatgtgacataacagaaatatactgccaagatttgaatataggtgattttataatgactaaggatggtcctgagatgttgatggatatttataaaaatggtaataaatcgaatatgtatgatttacaattatcagaaggctctaataaacaatactacacaaatgatatacttagtcataattcactttggatgcaa

SEQ ID NO: 110 - protein, 56 aa, Artificial Sequence
  IntN AceL-1_gp46-1
CVDESTLIDVQIIDFEPNLENLEFLDKTDEGKRIFLYIKKSNKSLYEKIEKFRKGQ

SEQ ID NO: 111 - protein, 122 aa, Artificial Sequence
  IntC AceL-1_gp46-1
MLTLKIGDLYELSKNINILESDIRVSTPGGLKKVFAVDITAKNSDVFSIKVNKHELLCSPDHLIRSEDMWVKSKDLKINSVIDTKYGKLTVKEISILDIKSDLMDLHVDGSEYYTNDIISHN

SEQ ID NO: 112 - protein, 5 aa, Artificial Sequence
  IntN AceL-1_gp46-1 flanking sequence
NGSGK

SEQ ID NO: 113 - protein, 5 aa, Artificial Sequence
  IntC AceL-1_gp46-1 flanking sequence
SSLLD

SEQ ID NO: 114 - DNA, 186 bp, Artificial Sequence
  DNA  IntN  AceL-1_gp46-1
aatggttctggtaagtgtgttgatgagtcaacactaatagatgtacaaataattgattttgagcctaatttagaaaatttagaatttttagacaaaacggatgagggaaagaggatttttctatatataaagaaatctaataaatccttgtatgaaaaaattgaaaaatttagaaaaggtcaataa

SEQ ID NO: 115 - DNA, 381 bp, Artificial Sequence
  DNA  IntC AceL-1_gp46-1
atgttaacattaaagataggtgatttatatgaattatcaaaaaatataaatattttagaatcagacatccgtgtatctaccccaggtggattgaaaaaggtttttgctgttgatataacagcaaaaaatagcgatgtgttttctataaaagttaataaacatgaactactttgctcaccagatcatctaataagatcagaagatatgtgggttaaatctaaagatttaaaaataaattccgtaatagatacaaaatatggcaaacttactgttaaggagatatcaattttggatataaagagtgatttgatggatttacatgtggatggtagtgaatattacactaatgatataattagtcacaactcatcactattagat

SEQ ID NO: 116 - protein, 15 aa, Artificial Sequence
  IntN VidaL_T4Lh-1
CVHPDTKVTIRRKLC

SEQ ID NO: 117 - protein, 155 aa, Artificial Sequence
  IntC VidaL_T4Lh-1
MKELLDLYTEKEINKLLERYTIDQIIDYSQPHVVSVGSIKEEMDSGNFIFVDSPDGYVAVSDFVDKGNFEEYRFTYDKKIIRTNEGHLFQTHLGWETSKNLYKMYLAGHPIYILHKNGGYKKIDIEKTGNVIPIVDIVVEHKNHRYYTDGLSSHN

SEQ ID NO: 118 - protein, 5 aa, Artificial Sequence
  IntN VidaL_T4Lh-1 flanking sequence
VFLAS

SEQ ID NO: 119 - protein, 5 aa, Artificial Sequence
  IntC VidaL_T4Lh-1 flanking sequence
TNVGK

SEQ ID NO: 120 - DNA, 63 bp, Artificial Sequence
  DNA  IntN  VidaL_T4Lh-1
gtatttttggctagttgtgtgcatccagatacaaaagtaacaattcgtagaaaactttgttag

SEQ ID NO: 121 - DNA, 480 bp, Artificial Sequence
  DNA IntC VidaL_T4Lh-1
atgaaagaattgcttgacttatacacagaaaaagaaataaataaattattagaaagatacacaatagaccagattatagactactcacaacctcatgtggtttctgtgggtagtataaaagaagaaatggattcaggaaatttcatttttgttgacagcccagatggttacgttgctgttagtgattttgtagacaaaggaaactttgaagaatataggtttacatatgataaaaaaataatccgaacaaacgaaggtcacttattccaaacacatttgggttgggagacttctaagaatttatataaaatgtacttagctggtcaccccatttatatattgcataaaaatggtggttataaaaagattgatatagaaaagaccggaaacgtgattcctatcgttgatattgtggtggaacacaaaaaccatagatattatacggatggattgtccagccataacacaaatgtgggcaag

SEQ ID NO: 122 - protein, 16 aa, Artificial Sequence
  IntN VidaL_UvsX-2
CLPKEAVVQIRLTKKG

SEQ ID NO: 123 - protein, 123 aa, Artificial Sequence
  IntC VidaL_UvsX-2
MIEEKKVTVQELRELYLSGEYTIEIDTPDGYQTIGKWFDKGVLSMVRVATATYETVCAFNHMIQLADNTWVQACELDVGVDIQTAAGIQPVMLVEDTSDAECYDFEVMHPNHRYYGDGIVSHN

SEQ ID NO: 124 - protein, 5 aa, Artificial Sequence
  IntN VidaL_UvsX-2 flanking sequence
SGKSY

SEQ ID NO: 125 - protein, 5 aa, Artificial Sequence
  IntC VidaL_UvsX-2 flanking sequence
AGESG

SEQ ID NO: 126 - DNA, 66 bp, Artificial Sequence
  DNA  IntN  VidaL_UvsX-2
gctggcgaaagtggctgtttgccaaaagaagcagtagtacagattcgattaacaaaaaaaggctag

SEQ ID NO: 127 - DNA, 384 bp, Artificial Sequence
  DNA  IntC  VidaL_UvsX-2
atgattgaagaaaagaaagtaacagtacaagagcttagagagctatatctcagcggcgagtatactattgagattgacacaccggacggatatcagactatcggaaaatggtttgacaaaggggtattgtccatggttagagttgccacagccacttacgaaacagtgtgtgcatttaatcatatgattcaactggctgacaatacgtgggtacaagcctgtgagttagatgtaggagtagatatacaaacggcggcaggcatccagcctgttatgttagtcgaagatacaagtgatgcagagtgttacgattttgaagtcatgcatccgaatcatagatattacggtgacggaattgtaagccataactcggggaaaagttat

SEQ ID NO: 128 - protein, 28 aa, Artificial Sequence
  IntN VidaL_TerL-6-1
SLAHETIVSINDNNTLTSMCIGDLYDYM

SEQ ID NO: 129 - protein, 132 aa, Artificial Sequence
  IntC VidaL_TerL-6-1
MDYHSNQVSRIFGVGMSKVHLGFKKNTKNLKVLTPNGHEEFYGINKIRVDEYIRIKFKEHKEIRCSIDHPFIQENDLPIKAKHIDKSKHIKCIDGFTTLEYSHVVNKQIELYDIVNSGSEYIYFSNGILSHN

SEQ ID NO: 130 - protein, 5 aa, Artificial Sequence
  IntN VidaL_TerL-6-1 flanking sequence
SVEYE

SEQ ID NO: 131 - protein, 5 aa, Artificial Sequence
  IntC VidaL_TerL-6-1 flanking sequence
CKFMG

SEQ ID NO: 132 - DNA, 102 bp, Artificial Sequence
  DNA  IntN  VidaL_TerL-6-1
tccgtggaatatgagagtttggcacacgaaactatagtaagtataaatgataataacacactaacaagtatgtgcattggagatttatatgactatatgtaa

SEQ ID NO: 133 - DNA, 411 bp, Artificial Sequence
  DNA Intc  VidaL_TerL-6-1
ttggattaccactctaatcaagtgtctcgaatttttggagttggtatgagcaaagtacatctagggtttaaaaagaacactaaaaatttaaaagtgttaacaccaaatggacacgaagaattctacggaataaacaaaatacgtgtcgatgaatatatacgaataaaattcaaagaacataaagaaatacgttgctcgattgaccacccgtttatacaagaaaatgatttaccaataaaagcaaaacatattgataaaagcaaacatataaaatgtattgatggatttactactttagagtattcgcatgttgttaataaacaaattgaactatatgatattgtaaactctggtagtgagtacatatatttttctaatgggatattaagtcacaactgtaaattcatgggt

SEQ ID NO: 134 - protein, 30 aa, Artificial Sequence
  IntN TerL-7-1_VidaL
CLWGASTVNVFDSLTGKNIDIKLEDLYQKL

SEQ ID NO: 135 - protein, 114 aa, Artificial Sequence
  IntC TerL-7-1_VidaL
MESYTFRKNTRYKIMTPAGYQNFGGIRKLNKNVHYIVELSNKKILKCSTTHPFIYNDREIFANKLKVGSLLDSTSKKKISVISIELDKSKIDLYDIVEVNNGNIFNVDGIVSHN

SEQ ID NO: 136 - protein, 5 aa, Artificial Sequence
  IntN TerL-7-1_VidaL flanking sequence
SQECD

SEQ ID NO: 137 - DNA, 1 bp, Artificial Sequence
  IntC TerL-7-1_VidaL flanking sequence
c

SEQ ID NO: 138 - DNA, 108 bp, Artificial Sequence
  DNA IntN TerL-7-1_VidaL
tcccaagaatgcgattgtttgtggggcgcatctactgtaaatgtatttgatagtttaactggaaaaaacattgatataaaactcgaagatttgtatcaaaaactttaa

SEQ ID NO: 139 - DNA, 345 bp, Artificial Sequence
  DNA  IntC  TerL-7-1_VidaL
atggaatcatatacttttagaaaaaacacaagatataaaataatgacaccagcaggatatcaaaactttggtggtattagaaaattgaataaaaatgtacattatatagttgaattatccaataaaaaaatattaaaatgttcaactacacatccatttatttataatgatagagagatatttgcaaataaattaaaagtcggtagtttacttgatagtactagtaaaaagaaaatttcagtaatatcaattgaattagataaatcaaaaatagatttatatgatatagtagaagtaaataatggtaatatttttaatgtagatggtattgtttcacataattgt

SEQ ID NO: 140 - protein, 31 aa, Artificial Sequence
  IntN VidaL_TerL-3
CVSASTIITLQDTHGNIFDSQIGDLYNTIGK

SEQ ID NO: 141 - protein, 114 aa, Artificial Sequence
  IntC VidaL_TerL-3
MSKIFKENTNGYKVLTPAGFQDFAGVSMMGIKPLLRLEFERGAYVECTYDHKFYIDLETCKPAQDIAVGNTVVTSEGDIKLLNKIELGYSEPVYDLIQVEGGHRYYTNKILSSN

SEQ ID NO: 142 - protein, 5 aa, Artificial Sequence
  IntN VidaL_TerL-3 flanking sequence
RREYG

SEQ ID NO: 143 - protein, 5 aa, Artificial Sequence
  IntC VidaL_TerL-3 flanking sequence
CEFLV

SEQ ID NO: 144 - DNA, 111 bp, Artificial Sequence
  DNA  IntN  VidaL_TerL-3
cgtcgtgagtacggttgtgtgagcgcatctacaatcattactctccaagacacacacggtaatatatttgactcacaaataggcgacttgtacaatacgataggtaaataa

SEQ ID NO: 145 - DNA, 357 bp, Artificial Sequence
  DNA  IntC  VidaL_TerL-3
atgagcaagatttttaaagagaatactaatggatataaggtgttaacaccagcggggtttcaagactttgctggtgttagcatgatgggaataaaaccgttgcttcggctagagttcgagcgaggcgcctacgtcgaatgcacctacgatcataaattttacatagacctagaaacttgtaagccagcccaagacattgcagtaggaaacactgtggttacttctgagggtgatataaaattactcaacaaaatagaactgggttattcagaacctgtttatgatcttatacaagttgaaggcggccaccgatattacacaaacaaaatactcagctcaaattgcgaatttttagta

SEQ ID NO: 146 - protein, 32 aa, Artificial Sequence
  IntN VidaL_TerL-1
CVQADTKYTIRNKISGDVLNVTAEEFHKMQKK

SEQ ID NO: 147 - protein, 121 aa, Artificial Sequence
  IntC VidaL_TerL-1
MKLSNFTNRKFIETIDASEWEVETCEGFKPIISSNKTIEYVVYKIELENGLSIKCADTHILIDKNLQEIYAKDSFNKIIFTKFGNSKVISVETLNISENMYDLSVDSEDHTYYTDDILSHN

SEQ ID NO: 148 - protein, 5 aa, Artificial Sequence
  IntN VidaL_TerL-1 flanking sequence
RQQGK

SEQ ID NO: 149 - protein, 5 aa, Artificial Sequence
  IntC VidaL_TerL-1 flanking sequence
TTTAA

SEQ ID NO: 150 - DNA, 114 bp, Artificial Sequence
  DNA  IntN VidaL_TerL-1
agacaacaaggtaagtgcgttcaagcagacactaaatacactataagaaacaaaattagtggtgatgtgttaaatgttacagcagaagaattccacaaaatgcagaaaaaataa

SEQ ID NO: 151 - DNA, 378 bp, Artificial Sequence
  DNA  IntC VidaL_TerL-1
atgaagctatccaatttcaccaatagaaaatttatagaaacaattgatgctagtgaatgggaagtagaaacatgcgaaggtttcaaacccatcattagttcaaataaaactattgaatatgtagtctataaaattgaactagaaaatggattatctattaaatgtgcagacactcatattttaatagataaaaatttgcaagaaatttatgcaaaagatagttttaataaaataatatttacaaagttcggaaactcaaaagttatttccgtagaaactttaaatatatctgaaaatatgtatgatctttctgttgattcagaagatcacacatactatacagatgatatcttatcacataataccacgaccgccgca

SEQ ID NO: 152 - protein, 32 aa, Artificial Sequence
  IntN VidaL_gp46-1
CLCINTIVKVKNTKTGVIYETTIGELYNGAME

SEQ ID NO: 153 - protein, 124 aa, Artificial Sequence
  IntC VidaL_gp46-1
MSTISQTVNRKFVNSFSLVDLEIETDSGWQPVTDIHKTIPYTVWHIETQSGLTLDCADTHILFDHNYNEIFVKDIIPNQTKIISKHGPELVLTVIEQSQQENMFDLTVDHPDHRFYSNNILSHN

SEQ ID NO: 154 - protein, 5 aa, Artificial Sequence
  IntN VidaL_gp46-1 flanking sequence
NGTGK

SEQ ID NO: 155 - protein, 5 aa, Artificial Sequence
  IntC VidaL_gp46-1 flanking sequence
TTVIN

SEQ ID NO: 156 - DNA, 114 bp, Artificial Sequence
  DNA  IntN  VidaL_gp46-1
aacggaacgggcaagtgcctttgtataaatactattgtaaaagtaaaaaacaccaaaactggggtaatttacgaaactacaataggagaattatacaatggcgcgatggaataa

SEQ ID NO: 157 - DNA, 387 bp, Artificial Sequence
  DNA  IntC VidaL_gp46-1
atgtctacaatttctcaaacagtaaatagaaaatttgttaatagtttcagcttagttgatcttgaaatcgaaacagattctggatggcagcctgttactgacatacacaagactattccatacacagtttggcatatcgaaacccaaagcgggctgactcttgactgtgccgacactcatatcttgtttgatcacaactacaacgagatctttgtcaaagatataatacctaaccagactaagataatatctaagcacggtcctgaattagtattaacagtaatcgaacagtctcagcaagaaaatatgtttgatctaacagttgatcatcctgatcatcgtttttactcaaacaatatcttatctcacaataccacagtgataaat

SEQ ID NO: 158 - protein, 43 aa, Artificial Sequence
  IntN VidaL_gp41-1
CVIAETEVKIIELYNIDSFIKTGILNSQGVVSWEETSSHGRTR

SEQ ID NO: 159 - protein, 154 aa, Artificial Sequence
  IntC VidaL_gp41-1
MNLLDKRVEWLSKWYPVDKLQQLSEDKLVLLYNNSQPKKVRMGSLEGVPTSSYRISSPDGYVTAHAWRNKGTKECVTLTTDSGNSITASTDHFFEMSDGKWKYAGCLFPGQCISTESGTETVTSVVAAGKHTVYDFYIDHENHRYYTNGISSHN

SEQ ID NO: 160 - protein, 5 aa, Artificial Sequence
  IntN VidaL_gp41-1 flanking sequence
IFAGG

SEQ ID NO: 161 - protein, 5 aa, Artificial Sequence
  IntC VidaL_gp41-1 flanking sequence
SGAGK

SEQ ID NO: 162 - DNA, 147 bp, Artificial Sequence
  DNA I IntN  VidaL_gp41-1
atatttgccggcggatgtgtaattgccgaaactgaggtaaaaataattgaattatacaacattgacagctttattaaaacaggaatacttaacagccaaggggtcgtttcgtgggaagaaacttcttcccacggtcgtactcgatag

SEQ ID NO: 163 - DNA, 477 bp, Artificial Sequence
  DNA IntC VidaL_gp41-1
atgaatttactagataagagagttgagtggttatctaagtggtacccagtagacaagttacaacaattatcagaagacaagttggtactcctatataacaatagtcagccaaaaaaagtccgcatgggatcgttggaaggtgttccgaccagcagttatcggatcagcagccccgacgggtatgttaccgcacatgcctggcgaaataaaggaactaaagagtgtgttactctaaccacagactctggaaattctataactgctagcacagatcattttttcgaaatgtccgacggcaagtggaaatatgcaggctgtttgtttccaggacagtgcattagcacagaatccggcaccgaaacagtgaccagcgtagtagcagcaggtaagcacacagtatacgacttctacatcgatcatgaaaatcacagatactataccaatggaataagcagtcataactctggtgcaggcaaa

SEQ ID NO: 164 - protein, 29 aa, Artificial Sequence
  IntN GS013_ter-3
CLGGDTEIEILDDNGIVQKTSMENLYERL

SEQ ID NO: 165 - protein, 103 aa, Artificial Sequence
  IntC GS013_ter-3
MFKINKNIKVKTPDGFKDFSGIQKVYKPFYHWIIFDDGSEIKCSDNHSFGKEKIKASTIKVDDILQEKKVLYNEIVEEGIYLYDLLDVGEDNLYYSNNIVSHN

SEQ ID NO: 166 - protein, 5 aa, Artificial Sequence
  IntN GS013_ter-3 flanking sequence
RVEFE

SEQ ID NO: 167 - protein, 5 aa, Artificial Sequence
  IntC GS013_ter-3 flanking sequence
CEFLG

SEQ ID NO: 168 - DNA, 105 bp, Artificial Sequence
  DNA  IntN  GS013_ter-3
cgggttgagtttgaatgtttgggtggtgatacagagattgaaattttggatgataatggaattgtacaaaaaacttctatggaaaatttatatgaacgattgtga

SEQ ID NO: 169 - DNA, 324 bp, Artificial Sequence
  DNA IntC GS013_ter-3
atgtttaagattaataaaaatattaaagtaaaaacacctgatggatttaaagatttttcaggaatacaaaaagtttataaacctttttaccattggataatatttgatgacggatcagaaataaaatgctccgataatcattctttcggaaaagaaaaaattaaggcatcaacaattaaagttgatgatattttacaagaaaagaaagtattatataatgaaatagtagaagaaggaatttatctttatgatttacttgatgttggcgaagacaatctttactattcaaacaatatagtatcacacaactgcgagttcttgggt

SEQ ID NO: 170 - protein, 29 aa, Artificial Sequence
  IntN GS013_ter-2
CLGGDTEIEILDDNGIVQKTSMENLYERL

SEQ ID NO: 171 - protein, 108 aa, Artificial Sequence
  IntC GS013_ter-2
MSVGKMFKINKNIKVKTPDGFKDFSGIQKVYKPFYHWIIFDDGSEIKCSDNHSFGKEKIKASTIKVDDILQEKKVLYNEIVEEGIYLYDLLDVGEDNLYYSNNIVSHN

SEQ ID NO: 172 - protein, 5 aa, Artificial Sequence
  IntN GS013_ter-2 flanking sequence
RVEFE

SEQ ID NO: 173 - protein, 5 aa, Artificial Sequence
  IntC GS013_ter-2 flanking sequence
CEFLG

SEQ ID NO: 174 - DNA, 105 bp, Artificial Sequence
  DNA  IntN  GS013_ter-2
cgtgttgagtttgaatgtttgggtggtgatacagagattgaaattttggatgataatggaatagtacaaaaaacttctatggaaaatttatatgaacgattgtga

SEQ ID NO: 175 - DNA, 339 bp, Artificial Sequence
  DNA I IntC  GS013_ter-2
atgagtgttggaaaaatgtttaagattaataaaaatattaaagtaaaaacacctgatggatttaaagatttttcaggaatacaaaaagtttataaacctttttaccattggataatatttgatgacggatcagaaataaaatgctccgataatcattctttcggaaaagaaaaaattaaggcatcaacaattaaagttgatgatattttacaagaaaagaaagtattatataatgaaatagtagaagaaggaatttatctttatgatttacttgatgttggcgaagacaatctttactattcaaacaatatagtatcacacaactgcgaattcttaggt

SEQ ID NO: 176 - protein, 39 aa, Artificial Sequence
  IntN GS013_ter-1
CFNTNTTVRLRNKLTGEIIEVTIGEFYEKIKKESNTDLP

SEQ ID NO: 177 - protein, 115 aa, Artificial Sequence
  IntC GS013_ter-1
MSKFIEEXXTDEWEVETPSGWQSFSGVGKTIEYEEWEVVTETGKSLICADKHILLNDKWQEVYCEDCSIDDCIQTKNXAEKILQLKKTSRIXNMYDLLDVDNGNIFYSNEIVSHN

SEQ ID NO: 178 - protein, 5 aa, Artificial Sequence
  IntN GS013_ter-1 flanking sequence
RQTGK

SEQ ID NO: 179 - protein, 5 aa, Artificial Sequence
  IntC GS013_ter-1 flanking sequence
STTVV

SEQ ID NO: 180 - DNA, 135 bp, Artificial Sequence
  DNA  IntN  GS013_ter-1
cgtcagacgggtaaatgttttaatacaaatacaacggtaaggttaaggaataaacttactggagaaattattgaagtgactattggagaattttatgaaaaaatcaagaaagaaagtaatactgatttgccttga

SEQ ID NO: 181 - DNA, 360 bp, Artificial Sequence
  DNA IntC GS013_ter-1
atgtctaaatttattgaagaartamaaactgatgaatgggaagtagaaactccttctggatggcaatctttttctggggtaggaaaaactatagaatatgaagaatgggaggttgtaaccgaaactggaaaatctcttatatgtgcagataaacacatcttattaaatgataaatggcaagaagtttattgtgargattgttccattgatgactgtatacaaacaaaaaatkgcgcagaaaaaatattacaattaaaaaaaacatcaagaattyytaatatgtatgatcttcttgatgttgataatggtaatatattttacagtaatgaaatagtttcacacaattctacaactgttgtc

SEQ ID NO: 182 - protein, 31 aa, Artificial Sequence
  IntN GS020_ter-7
CVDGSSIITIKNKETNLIEKITIEELYNKLL

SEQ ID NO: 183 - protein, 108 aa, Artificial Sequence
  IntC GS020_ter-7
MKTNTKYEILGPEGFVDFKGIQKLKKKTRQIFFECGLTLRASYNHKIYDYFGDEIIIKDVVIGSKIKSHNGYLIVNSIKDFDYESDVYDVIDSGDSHLYYTNNIVSHN

SEQ ID NO: 184 - protein, 5 aa, Artificial Sequence
  IntN GS020_ter-7 flanking sequence
SQELE

SEQ ID NO: 185 - protein, 5 aa, Artificial Sequence
  IntC GS020_ter-7 flanking sequence
CNFLG

SEQ ID NO: 186 - DNA, 111 bp, Artificial Sequence
  DNA  IntN GS020_ter-7
tcgcaggaattagagtgtgttgatggttcctcaattataactataaaaaacaaagagacaaatttaatagaaaaaataacaatagaagaattatacaataaattgttatag

SEQ ID NO: 187 - DNA, 339 bp, Artificial Sequence
  DNA  IntC GS020_ter-7
atgaaaactaacacaaaatatgaaattttaggtcctgaaggattcgtcgatttcaaaggtattcaaaaattaaaaaagaaaactagacaaattttttttgagtgtggactaacattacgagcaagttataaccacaagatttacgattattttggggatgaaattataattaaagacgtagttattggtagtaaaatcaaatcacataatggttatttaattgttaatagtatcaaggattttgattatgaaagtgacgtatatgacgttattgattcaggtgattcacatttatactacacaaacaacattgtttctcataattgtaattttcttggg

SEQ ID NO: 188 - DNA, 75 bp, Artificial Sequence
  IntN WT AceL-TerL-11 original DNA sequence
tgtgtttatggtgatacaatggttgaaacagaagatggtaaaataaaaatagaagatttatataaaaggttggca

SEQ ID NO: 189 - DNA, 315 bp, Artificial Sequence
  IntC WT AceL-TerL-11 original DNA sequence
atgtttagaactaatacaaataatataaaaatattaagtccaaatggattttctaattttaatggtattcaaaaggttgaaagaaacctttatcaacacattatctttgatgatgatactgaaataaaaacttccattaatcatccttttggtaaagataaaatattagcaagagatgtaaaagtaggagattatttaaatagtaaaaaggtattatataatgagttggttaatgaaaatatatttttatatgatcctataaatgtagaaaaagaaagtttatatattactaatggtgttgtttctcataattgt

SEQ ID NO: 190 - DNA, 75 bp, Artificial Sequence
  IntN WT AceL-TerL-11 codon optimised DNA sequence
tgcgtgtatggcgatactatggtggaaaccgaagatggcaaaattaaaattgaagatctgtataaacgtctggcc

SEQ ID NO: 191 - DNA, 315 bp, Artificial Sequence
  IntC WT AceL-TerL-11 codon optimised DNA sequence
ggcatgtttcgtaccaacaccaacaacattaaaattctgagcccgaacggctttagcaactttaacggcattcagaaagtggaacgtaacctgtatcagcatattatttttgatgatgataccgaaattaaaaccagcattaaccatccgtttggcaaagataaaattctggcgcgtgatgtgaaagtgggcgattatctgaacagcaaaaaagtgctgtataacgaactggtgaacgaaaacatttttctgtatgatccgattaacgtggaaaaagaaagcctgtatattaccaacggcgtggtgagccataac

SEQ ID NO: 192 - DNA, 111 bp, Artificial Sequence
  DNA  IntN  GS033_TerA-6 codon optimised DNA sequence
agcattagccaggaatcctatatcaacattgaggtgaacgggaaagtggaaaccatcaaaatcggcgacctgtataaaaaactgtccttcaacgagcgtaaattcaacgag

SEQ ID NO: 193 - DNA, 345 bp, Artificial Sequence
  DNA  IntC  GS033_TerA-6 codon optimised DNA sequence
atgaaactgccggagagcgtggtgaaaaacaacatcaacctgaaaatcgaaaccccgtatggctttgagaacttctatggtgtgaacaaaatcaaaaaagacaaatatatccacctggagtttaccaacggcgaaaaactgaaatgctccctggatcatcctctgtctaccattgacggcatcgttaaagcgaaagatctggacaaatataccgaggtctatacgaaatttggtggctgctttctgaaaaaatccaaagtgatcaacgagtccatcgagctgtatgatatcgtgaactctgggctgaaacacctgtattattccaacaatattatcagtcacaac

SEQ ID NO: 194 - protein, 104 aa, Artificial Sequence; identical to SEQ ID NO: 81
  IntC Wt AceL-TerL3

SEQ ID NO: 195 - protein, 104 aa, Artificial Sequence; identical to SEQ ID NO: 87
  IntC WTAceL Terl4

SEQ ID NO: 196 - protein, 104 aa, Artificial Sequence; identical to SEQ ID NO: 93
  IntC WT AceL-TerL5

SEQ ID NO: 197 - protein, 353 aa, Artificial
  SBP-(VidaL_T4Lh-1)C-Trx-His6
MDEKTTGWRGGHVVEGLAGELEQLRARLEHHPQGQREPGASGGGGSSSNNNNNNNNNNLGIEGRISEFKELLDLYTEKEINKLLERYTIDQIIDYSQPHVVSVGSIKEEMDSGNFIFVDSPDGYVAVSDFVDKGNFEEYRFTYDKKIIRTNEGHLFQTHLGWETSKNLYKMYLAGHPIYILHKNGGYKKIDIEKTGNVIPIVDIVVEHKNHRYYTDGLSSHNTNVGGSGGTGMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSVDRSHHHHHH

SEQ ID NO: 198 - DNA, 870 bp, Artificial
  SBP-(VidaL_T4Lh-1)C-Trx-His6  nt
atgggcactagtaaagaactgctggatctgtataccgaaaaagaaattaacaaactgctggaacgctataccattgatcagattattgattatagccagccgcatgtggtgagcgtgggcagcattaaagaagaaatggatagcggcaactttatttttgtggatagcccggatggctatgtggcggtgagcgattttgtggataaaggcaactttgaagaatatcgctttacctatgataaaaaaattattcgcaccaacgaaggccatctgtttcagacccatctgggctgggaaaccagcaaaaacctgtataaaatgtatctggcgggccatccgatttatattctgcataaaaacggcggctataaaaaaattgatattgaaaaaaccggcaacgtgattccgattgtggatattgtggtggaacataaaaaccatcgctattataccgatggcctgagcagccataacaccaacgtgggcggcagcggcggtaccggtatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggctctgtcgacagatctcatcaccatcaccatcactaa

SEQ ID NO: 199 - protein, 18 aa, Artificial
  (VidaL_T4Lh-1)N peptide
LASCVHPDTKVTIRRKLC

SEQ ID NO: 200 - protein, 289 aa, Artificial
  (VidaL_T4Lh-1)C-Trx-His6 aa
MGSTKELLDLYTEKEINKLLERYTIDQIIDYSQPHVVSVGSIKEEMDSGNFIFVDSPDGYVAVSDFVDKGNFEEYRFTYDKKIIRTNEGHLFQTHLGWETSKNLYKMYLAGHPIYILHKNGGYKKIDIEKTGNVIPIVDIVVEHKNHRYYTDGLSSHNTNVGGSGGTGMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSVDRSHHHHHH

SEQ ID NO: 201 - DNA, 870 bp, Artificial; identical to SEQ ID NO: 198
  (VidaL_T4Lh-1)C-Trx-His6 nt

SEQ ID NO: 202 - protein, 467 aa, Artificial
  MBP-(VidaL_T4Lh-1)N-linker-SBP aa
MGTKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRGTLELASCVHPDTKVTIRRKLCSEFGSPRKVIKMESEERSMDEKTTGWRGGHVVEGLAGELEQLRARLEHHPQGQREP

SEQ ID NO: 203 - DNA, 1404 bp, Artificial
  MBP-(VidaL_T4Lh-1)N-linker-SBP
atgggtaccaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggggtacgctcgagctggcgagctgcgtgcatccggataccaaagtgaccattcgccgcaaactgtgcagcgaattcggatccccgcgtaaagtgattaaaatggaatctgaagaaagatctatggacgaaaaaaccaccggttggcgtggtggtcacgttgttgaaggtctggctggtgaactggaacagctgcgtgctcgtctggaacaccacccgcagggtcagcgtgaaccctaa

SEQ ID NO: 204 - protein, 256 aa, Artificial
  (VidaL_UvsX-2)C-Trx-His6 aa
MGTSIEEKKVTVQELRELYLSGEYTIEIDTPDGYQTIGKWFDKGVLSMVRVATATYETVCAFNHMIQLADNTWVQACELDVGVDIQTAAGIQPVMLVEDTSDAECYDFEVMHPNHRYYGDGIVSHNSGKGSGGTGMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSVDRSHHHHHH

SEQ ID NO: 205 - DNA, 771 bp, Artificial
  (VidaL_UvsX-2)C-Trx-His6  nt
atgggcactagtattgaagaaaaaaaagtgaccgtgcaggaactgcgcgaactgtatctgagcggcgaatataccattgaaattgataccccggatggctatcagaccattggcaaatggtttgataaaggcgtgctgagcatggtgcgcgtggcgaccgcgacctatgaaaccgtgtgcgcgtttaaccatatgattcagctggcggataacacctgggtgcaggcgtgcgaactggatgtgggcgtggatattcagaccgcggcgggcattcagccggtgatgctggtggaagataccagcgatgcggaatgctatgattttgaagtgatgcatccgaaccatcgctattatggcgatggcattgtgagccataacagcggcaaaggcagcggcggtaccggtatgagcgataaaattattcacctgactgacgacagttttgacacggatgtactcaaagcggacggggcgatcctcgtcgatttctgggcagagtggtgcggtccgtgcaaaatgatcgccccgattctggatgagatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggctctgtcgacagatctcatcaccatcaccatcactaa

SEQ ID NO: 206 - protein, 20 aa, Artificial
  VidaL_UvsX-2)N peptide
ESGCLPKEAVVQIRLTKKGA

(19) United States
(12) Patent Application Publication
(10) Pub. No.: US 2016/0319287 A1
(43) Pub. Date: Nov. 3, 2016

(54) ATYPICAL INTEINS

(71) Applicants: WESTFÄLISCHE WILHELMS-UNIVERSITÄT MÜNSTER, Münster (DE); YEDA RESEARCH & DEVELOPMENT CO. LTD., Rehovot (IL)

(72) Inventors: Henning Dieter MOOTZ, Münster (DE); Ilka THIEL, Münster (DE); Gerrit VOLKMANN, Münster (DE); Shmuel PIETROKOVSKI, Rehovot (IL); Anne-Lena BACHMANN, Münster (DE)

(73) Assignees: WESTFÄLISCHE WILHELMS-UNIVERSITÄT MÜNSTER, Münster (DE); YEDA RESEARCH & DEVELOPMENT CO. LTD., Rehovot (IL)

(21) Appl. No.: 15/103,839

(22) PCT Filed: Dec. 12, 2014
(86) PCT No.: PCT/EP2014/077578

§ 371 Date: Jun. 10, 2016

(30) Foreign Application Priority Data
Dec. 12, 2013 (EP) 13196924.8

(51) Int. Cl. C12N 15/62; C12N 9/22; C07K 14/00; C12N 15/10; C07K 14/245; C07K 7/06

(52) U.S. Cl. CPC C12N 15/62; C07K 14/245; C07K 7/06; C07K 14/00; C12N 15/102; C12N 9/22; C07K 2319/92; C07K 2319/21; C12N 2795/10122

(57) ABSTRACT

The present invention relates to an isolated polypeptide comprising at least one intein or at least one intein fragment of said intein, wherein said intein is a naturally split intein with a N-terminal intein fragment split after 14-60 amino acids from the intein's N-terminal end and methods of using the same.

### BACKGROUND OF THE INVENTION

Inteins are internal protein sequences that excise themselves out of a precursor protein in an autocatalytic reaction called protein splicing. In protein trans-splicing the intein domain is split and located on two separate polypeptides. Protein trans-splicing catalysed by split inteins is a powerful technique to assemble a polypeptide backbone from two separate parts. During the reaction the N- and C-terminal intein fragments (also termed Int^{N} and Int^{C}) first associate and fold into the active intein domain and then link the flanking sequences, also termed the N- and C-terminal exteins (Ext^{N} and Ex^{C}), with a peptide bond while at the same time precisely excising the intein sequence. Apart of their homologous N- and C-terminal exteins, inteins will generally also excise themselves out of heterologous sequence flanks. Moreover, an intein is a self-contained entity, that is, it does not require any additional cofactors or energy sources to perform the protein splicing reaction.

The split intein based trans-splicing reaction has found various applications in basic protein research and biotechnology, e.g. for segmental isotope labelling of proteins, preparation of cyclic polypeptides, transgene expression, as well as more recently for chemical modification of proteins and protein semi-synthesis (R. Borra, J. A. Camarero, Biopolymers 2013; T. C. Evans, Jr., M. Q. Xu, S. Pradhan, Annu. Rev. Plant Biol. 2005, 56, 375; C. J. Noren, J. Wang, F. B. Perler, Angew. Chem. 2000, 112, 458; Angew. Chem. Int. Ed. Engl. 2000, 39, 450; M. Vila-Perello, T. W. Muir, Cell 2010, 143, 191-200; G. Volkmann, H. Iwai, Mol. Biosyst. 2010, 6, 2110; G. Volkmann, H. D. Mootz, Cell. Mol. Life. Sci. 2013, 70, 118).

However, split inteins are rare, and especially for chemical modification of proteins and protein semi-synthesis special properties are required. Specifically, one of the intein fragments should be as short as possible to facilitate its efficient and inexpensive assembly by solid-phase peptide synthesis. All naturally occurring split inteins reported so far show the break-point at the position of the homing endonuclease domain in the related contiguous maxi-inteins. This split site gives rise to an Int^{N} of about 100 amino acids (aa) and an Int^{C} of about 35 aa (I. Giriat, T. W. Muir, J. Am. Chem. Soc. 2003, 125, 7180-7181; H. Wu, Z. Hu, X. Q. Liu, Proc. Natl. Acad. Sci. USA 1998, 95, 9226). Split inteins with shorter Int^{N} or Int^{C} fragments have been created artificially from naturally contiguous inteins (J. H. Appleby, K. Zhou, G. Volkmann, X. Q. Liu, J. Biol. Chem. 2009, 284, 6194; A. S. Aranko, S. Zuger, E. Buchinger, H. lwai, PloS One 2009, 4, e5185; Y. T. Lee, T. H. Su, W. C. Lo, P. C. Lyu, S. C. Sue, PloS One 2012, 7, e43820; C. Ludwig, M. Pfeiff, U. Linne, H. D. Mootz, Angew. Chem. 2006, 118, 5343; Angew. Chem. Int. Ed. Engl. 2006, 45, 5218; W. Sun, J. Yang, X. Q. Liu, J. Biol. Chem. 2004, 279, 35281; G. Volkmann, X. Q. Liu, PloS One 2009, 4, e8381), but these generally show lower splicing yields and rates and tend to associate and fold less efficiently. Moreover, another drawback of known split inteins is a limited compatibility with diverse target proteins due to the solubility and expression issues of the recombinant split intein fusion constructs.

Hence, there exists need in the art for alternative split inteins that ameliorate or overcome the known problems.

### SUMMARY OF THE INVENTION

The present invention is based on the unexpected finding that specific inteins or fragments of said inteins have the property that the N-terminal intein fragment of said intein is split after only 14-60 amino acids from the intein's N-terminal end, with these inteins being naturally split inteins. These inteins provide for the shortest naturally occurring N-terminal intein fragments discovered so far. Moreover, they exhibit excellent splicing yields and rates.

Thus, in a first aspect, the present invention relates to an isolated polypeptide comprising at least one intein or at least one fragment of said intein, wherein said intein is a naturally split intein with a N-terminal intein fragment split after 14-60 amino acids from the intein's N-terminal end.

Due to the very short N-terminal parts, these inteins or their Int^{N} fragments, respectively, can be easily generated via solid peptide synthesis, which is faster, more reliable and robust than protein generation via recombinant protein expression techniques.

Hence, these novel split inteins are ideally suited for all kinds of efficient protein modifications.

Moreover, it was surprisingly found possible to further modify some of these natural split inteins to even increase splicing yields and rates and thus render the novel split inteins suited for an even wider range of applications and assay conditions.

In various aspects, the present invention relates to an isolated polypeptide as described above wherein said at least one intein or intein fragment is selected from the group comprising:
a) an N-terminal intein fragment having at least 70%, at least 80, at least 85%, at least 90%, at least 95% or 100% sequence identity with the amino acid sequence set forth in any one of SEQ ID Nos. 2, 3, 4, 5, 28, 38, 44, 50, 56, 62, 68, 74, 80, 86, 92, 98, 104, 110, 116, 122, 128, 134, 140, 146, 152, 158, 164, 170, 176 and 182, and/or
b) a C-terminal intein fragment having at least 70%, at least 80, at least 85%, at least 90%, at least 95% or 100% sequence identity with the amino acid sequence set forth in any one of SEQ ID Nos. 6, 27, 39, 45, 51, 57, 63, 69, 75, 81, 87, 93, 99, 105, 111, 117, 123, 129, 135, 141, 147, 153, 159, 165, 171, 177, 183, 194, 195 and 196, or
c) an intein having at least 70%, at least 80%, at least 85%, at least 90%, at least 95% or 100% sequence identity with the amino acid sequence set forth in SEQ ID NO:1 or SEQ ID NO:26.

In various further aspects, the present invention relates to an isolated polypeptide as described above, wherein said polypeptide comprises at least one N-terminal intein fragment and at least one C-terminal intein fragment, wherein
1) the at least one N-terminal intein fragment comprises or consists of the amino acid sequence set forth in SEQ ID NO:5 and the at least one C-terminal intein fragment comprises or consists of the amino acid sequence set forth in SEQ ID NO:6, or
2) the at least one N-terminal intein fragment comprises or consists of the amino acid sequence set forth in any one of SEQ ID Nos. 5, 28-33 and the at least one C-terminal intein fragment comprises or consists of the amino acid sequence set forth in SEQ ID NO:27, or
3) the at least one N-terminal intein fragment comprises or consists of the amino acid sequence set forth in SEQ ID NO:38 and the at least one C-terminal intein fragment comprises or consists of the amino acid sequence set forth in SEQ ID NO:39, or
4) the at least one N-terminal intein fragment comprises or consists of the amino acid sequence set forth in SEQ ID NO:44 and the at least one C-terminal intein fragment comprises or consists of the amino acid sequence set forth in SEQ ID NO:45, or
5) the at least one N-terminal intein fragment comprises or consists of the amino acid sequence set forth in SEQ ID NO:50 and the at least one C-terminal intein fragment comprises or consists of the amino acid sequence set forth in SEQ ID NO:51, or
6) the at least one N-terminal intein fragment comprises or consists of the amino acid sequence set forth in SEQ ID NO:56 and the at least one C-terminal intein fragment comprises or consists of the amino acid sequence set forth in SEQ ID NO:57, or
7) the at least one N-terminal intein fragment comprises or consists of the amino acid sequence set forth in SEQ ID NO:62 and the at least one C-terminal intein fragment comprises or consists of the amino acid sequence set forth in SEQ ID NO:63, or
8) the at least one N-terminal intein fragment comprises or consists of the amino acid sequence set forth in SEQ ID NO:68 and the at least one C-terminal intein fragment comprises or consists of the amino acid sequence set forth in SEQ ID NO:69, or
9) the at least one N-terminal intein fragment comprises or consists of the amino acid sequence set forth in SEQ ID NO:74 and the at least one C-terminal intein fragment comprises or consists of the amino acid sequence set forth in SEQ ID NO:75, or
10) the at least one N-terminal intein fragment comprises or consists of the amino acid sequence set forth in SEQ ID NO:80 and the at least one C-terminal intein fragment comprises or consists of the amino acid sequence set forth in SEQ ID NO:81, or
11) the at least one N-terminal intein fragment comprises or consists of the amino acid sequence set forth in SEQ ID NO:86 and the at least one C-terminal intein fragment comprises or consists of the amino acid sequence set forth in SEQ ID NO:87, or
12) the at least one N-terminal intein fragment comprises or consists of the amino acid sequence set forth in SEQ ID NO:92 and the at least one C-terminal intein fragment comprises or consists of the amino acid sequence set forth in SEQ ID NO:93, or
13) the at least one N-terminal intein fragment comprises or consists of the amino acid sequence set forth in SEQ ID NO:98 and the at least one C-terminal intein fragment comprises or consists of the amino acid sequence set forth in SEQ ID NO:99, or
14) the at least one N-terminal intein fragment comprises or consists of the amino acid sequence set forth in SEQ ID NO:104 and the at least one C-terminal intein fragment comprises or consists of the amino acid sequence set forth in SEQ ID NO:105, or
15) the at least one N-terminal intein fragment comprises or consists of the amino acid sequence set forth in SEQ ID NO:110 and the at least one C-terminal intein fragment comprises or consists of the amino acid sequence set forth in SEQ ID NO:111, or
16) the at least one N-terminal intein fragment comprises or consists of the amino acid sequence set forth in SEQ ID NO:116 and the at least one C-terminal intein fragment comprises or consists of the amino acid sequence set forth in SEQ ID NO:117, or
17) the at least one N-terminal intein fragment comprises or consists of the amino acid sequence set forth in SEQ ID NO:122 and the at least one C-terminal intein fragment comprises or consists of the amino acid sequence set forth in SEQ ID NO:123, or
18) the at least one N-terminal intein fragment comprises or consists of the amino acid sequence set forth in SEQ ID NO:128 and the at least one C-terminal intein fragment comprises or consists of the amino acid sequence set forth in SEQ ID NO:129, or
19) the at least one N-terminal intein fragment comprises or consists of the amino acid sequence set forth in SEQ ID NO:134 and the at least one C-terminal intein fragment comprises or consists of the amino acid sequence set forth in SEQ ID NO:135, or
20) the at least one N-terminal intein fragment comprises or consists of the amino acid sequence set forth in SEQ ID NO:140 and the at least one C-terminal intein fragment comprises or consists of the amino acid sequence set forth in SEQ ID NO:141, or
21) the at least one N-terminal intein fragment comprises or consists of the amino acid sequence set forth in SEQ ID NO:146 and the at least one C-terminal intein fragment comprises or consists of the amino acid sequence set forth in SEQ ID NO:147, or
22) the at least one N-terminal intein fragment comprises or consists of the amino acid sequence set forth in SEQ ID NO:152 and the at least one C-terminal intein fragment comprises or consists of the amino acid sequence set forth in SEQ ID NO:153, or
23) the at least one N-terminal intein fragment comprises or consists of the amino acid sequence set forth in SEQ ID NO:158 and the at least one C-terminal intein fragment comprises or consists of the amino acid sequence set forth in SEQ ID NO:159, or
24) the at least one N-terminal intein fragment comprises or consists of the amino acid sequence set forth in SEQ ID NO:164 and the at least one C-terminal intein fragment comprises or consists of the amino acid sequence set forth in SEQ ID NO:165, or
25) the at least one N-terminal intein fragment comprises or consists of the amino acid sequence set forth in SEQ ID NO:170 and the at least one C-terminal intein fragment comprises or consists of the amino acid sequence set forth in SEQ ID NO:171, or
26) the at least one N-terminal intein fragment comprises or consists of the amino acid sequence set forth in SEQ ID NO:176 and the at least one C-terminal intein fragment comprises or consists of the amino acid sequence set forth in SEQ ID NO:177, or
27) the at least one N-terminal intein fragment comprises or consists of the amino acid sequence set forth in SEQ ID NO:182 and the at least one C-terminal intein fragment comprises or consists of the amino acid sequence set forth in SEQ ID NO:183
28) the at least one N-terminal intein fragment comprises or consists of the amino acid sequence set forth in SEQ ID NO:2 and the at least one C-terminal intein fragment comprises or consists of the amino acid sequence set forth in SEQ ID NO:194, or
29) the at least one N-terminal intein fragment comprises or consists of the amino acid sequence set forth in SEQ ID NO:3 and the at least one C-terminal intein fragment comprises or consists of the amino acid sequence set forth in SEQ ID NO:195, or
30) the at least one N-terminal intein fragment comprises or consists of the amino acid sequence set forth in SEQ ID NO:4 and the at least one C-terminal intein fragment comprises or consists of the amino acid sequence set forth in SEQ ID NO:196.

In various further aspects, the present invention relates to an isolated polypeptide as described above wherein said polypeptide at the N-terminal end of the at least one N-terminal intein fragment and/or at the C-terminal end of the at least one C-terminal intein fragment further comprises a flanking amino acid sequence, wherein said flanking amino acid sequence is selected from:
1) in the case of SEQ ID NO:5 and/or 6 SEQ ID NO:25 and/or 26, respectively,
2) in the case of SEQ ID NO:5, 28-33 and/or 27 SEQ ID NO:34 and/or 35, respectively,
3) in the case of SEQ ID NO:38 and/or 39 SEQ ID NO:40 and/or 41, respectively,
4) in the case of SEQ ID NO:44 and/or 45 SEQ ID NO:46 and/or 47, respectively,
5) in the case of SEQ ID NO:50 and/or 51 SEQ ID NO:52 and/or 53, respectively,
6) in the case of SEQ ID NO:56 and/or 57 SEQ ID NO:58 and/or 59, respectively,
7) in the case of SEQ ID NO:62 and/or 63 SEQ ID NO:64 and/or 65, respectively,
8) in the case of SEQ ID NO:68 and/or 69 SEQ ID NO:70 and/or 71, respectively,
9) in the case of SEQ ID NO:74 and/or 75 SEQ ID NO:76 and/or 77, respectively,
10) in the case of SEQ ID NO:80 and/or 81 SEQ ID NO:82 and/or 83, respectively,
11) in the case of SEQ ID NO:86 and/or 87 SEQ ID NO:88 and/or 89, respectively,
12) in the case of SEQ ID NO:92 and/or 93 SEQ ID NO:94 and/or 95, respectively,
13) in the case of SEQ ID NO:98 and/or 99 SEQ ID NO:100 and/or 101, respectively,
14) in the case of SEQ ID NO:104 and/or 105 SEQ ID NO:106 and/or 107, respectively,
15) in the case of SEQ ID NO:110 and/or 111 SEQ ID NO:112 and/or 113, respectively,
16) in the case of SEQ ID NO:116 and/or 117 SEQ ID NO:118 and/or 119, respectively,
17) in the case of SEQ ID NO:122 and/or 123 SEQ ID NO:124 and/or 125, respectively,
18) in the case of SEQ ID NO:128 and/or 129 SEQ ID NO:130 and/or 131, respectively,
19) in the case of SEQ ID NO:134 and/or 135 SEQ ID NO:136 and/or 137, respectively,
20) in the case of SEQ ID NO:140 and/or 141 SEQ ID NO:142 and/or 143, respectively,
21) in the case of SEQ ID NO:146 and/or 147 SEQ ID NO:148 and/or 149, respectively,
22) in the case of SEQ ID NO:152 and/or 153 SEQ ID NO:154 and/or 155, respectively,
23) in the case of SEQ ID NO:158 and/or 159 SEQ ID NO:160 and/or 161, respectively,
24) in the case of SEQ ID NO:164 and/or 165 SEQ ID NO:166 and/or 167, respectively,
25) in the case of SEQ ID NO:170 and/or 171 SEQ ID NO:172 and/or 173, respectively,
26) in the case of SEQ ID NO:176 and/or 177 SEQ ID NO:178 and/or 179, respectively,
27) in the case of SEQ ID NO:182 and/or 183 SEQ ID NO:184 and/or 185, respectively.

In a further aspect, the present invention relates to an isolated polypeptide as described above, wherein the N-terminal intein fragment has (or comprises an amino acid sequence that has) at least 90%, at least 95% or 100% amino acid sequence identity with an amino acid sequence as set forth in any one of SEQ ID Nos. 5, 11 and 12, and/or wherein the C-terminal intein fragment has (or comprises an amino acid sequence that has) at least 90%, at least 95% or 100% amino acid sequence identity with an amino acid sequence set forth in any one of SEQ ID Nos. 6 and 13-18.

In a still further aspect, the invention relates to an isolated polypeptide as described above, wherein the N-terminal intein fragment has an amino acid sequence comprising or consisting of any one of the amino acid sequences set forth in SEQ ID Nos. 5, 11 and 12 and/or, wherein the C-terminal intein fragment has an amino acid sequence comprising or consisting of any one of the amino acid sequences set forth in SEQ ID Nos. 6 and 13-18.

In various further aspects, the invention also encompasses an isolated polypeptide as described above, wherein the polypeptide comprises an N-terminal intein fragment having the amino acid sequence set forth in SEQ ID NO:5 or SEQ ID NO:12 and a C-terminal intein fragment having the amino acid sequence set forth in SEQ ID NO:13.

In still other aspects, the invention relates to an isolated polypeptide as described above, wherein the N-terminal intein fragment has at least 90%, at least 95% or 100% amino acid sequence identity with an amino acid sequence as set forth in any one of SEQ ID Nos. 5, 28-33, and/or wherein the C-terminal intein fragment has at least 90%, at least 95% or 100% amino acid sequence identity with an amino acid sequence as set forth in SEQ ID NO:27.

In a further aspect, the present invention relates to an isolated polypeptide as described above, wherein the polypeptide further comprises at least one C-terminal extein and/or at least one N-terminal extein sequence.

In a further aspect, the present invention relates to two isolated polypeptides as described above or a combination of two polypeptides as described above or a composition comprising those, wherein the first isolated polypeptide comprises at least one heterologous N-terminal extein fused to an N-terminal intein fragment having at least 70%, at least 80%, at least 85%, at least 90%, at least 95%, at least 99% or 100% sequence identity to any one of the amino acid sequences set forth in SEQ ID Nos. 5, 28, 38, 44, 50, 56, 62, 68, 74, 80, 86, 92, 98, 104, 110, 116, 122, 128, 134, 140, 146, 152, 158, 164, 170, 176 and 182, and wherein the second isolated polypeptide comprises at least one C-terminal extein sequence fused to a C-terminal intein fragment having at least 70%, at least 80%, at least 85%, at least 90%, at least 95%, at least 99% or 100% sequence identity to any one of the amino acid sequences set forth in SEQ ID Nos. 6, 27, 39, 45, 51, 57, 63, 69, 75, 81, 87, 93, 99, 105, 111, 117, 123, 129, 135, 141, 147, 153, 159, 165, 171, 177 and 183.

In a further aspect, the present invention relates to an isolated polypeptide as described above, wherein the polypeptide or any one of the two polypeptides further comprises at least one component selected from a solubility factor, a marker, a linker, an epitope, an affinity tag, a fluorophore or a fluorescent protein, a toxic compound or protein and a small-molecule or a small-molecule binding protein.

In a further aspect, the present invention relates to an isolated nucleic acid molecule comprising a nucleotide sequence encoding for at least one polypeptide as described herein or a homolog, variant or complement thereof.

In a further aspect, the present invention relates to a method using an isolated polypeptide or a nucleic acid molecule as described above, wherein the method is selected from the group consisting of modification of a protein, protein lipidation, protein immobilization, protein backbone semi-synthesis, regioselective protein side chain modification and artificial control of protein splicing by light.

In a further aspect, the present invention relates to the use of a polypeptide or a nucleic acid molecule according as described above, wherein the use is selected from the group consisting of modification of a protein, protein lipidation, protein immobilization, protein backbone semisynthesis and use as molecular switch.

In a further aspect, the present invention relates to a kit comprising at least one polypeptide or a nucleic acid molecule as described above.

### BRIEF DESCRIPTION OF THE DRAWINGS

The invention will be better understood with reference to the detailed description when considered in conjunction with the non-limiting examples and the accompanying drawings.

In FIG. 1 the protein trans-splicing mediated by inteins is schematically depicted. In detail, the Int^{N} and Int^{C} fragments ligate their flanking sequences with a native peptide bond. These can either be their native exteins or unrelated peptides or proteins.

FIG. 2 shows a model of the of AceL-TerL (SEQ ID NO:1) sequence. In detail, the probable location of the split site is indicated by scissors and selected mutations are shown. Moreover, the active site is indicated by the dotted circle and the unstructured loop representing the position of the removed homing endonuclease domain is represented by the dashed line. It can be seen that the AceL-TerL inteins have a novel split site corresponding to a probable surface loop region of the intein with no defined secondary structure following β-strand 3 and α-helix 1.

FIG. 3 shows results of the characterization of the AceL-TerL intein (SEQ ID NO:1). Top: WT^{C} (SEQ ID NO:6)-Trx-His6 (15 μM) was incubated with pepWT^{N} (SEQ ID NO:5, 45 μM) at 25° C. for 24 h and the reaction mixture was analyzed by SDS-PAGE using UV illumination or Coomassie Brilliant Blue staining. Calculated molecular masses are: WT^{C}-Trx=26.4 kDa; SP=15.2 kDa; Int^{N}=2.9 kDa; Int^{C}=12.2 kDa; C-terminal cleavage product (Trx)=14.1 kDa. Bottom: Time-courses of SP and C-terminal cleavage product (C-cl.) formation at the indicated temperatures.

FIG. 4 demonstrates the temperature dependence of the AceL-TerL intein (SEQ ID NO:1). In detail, the intein construct WT^{C}-Trx-His6 (15 μM; =educt) was incubated with pepWT^{N} (45 μM) at 37° C., 25° C., and 8° C. Aliquots were removed at indicated time points and analysed by SDS-PAGE using Coomassie Brilliant Blue staining. Positions of expected protein products are indicated. The Int^{C} fragment can appear as a double band due to succinimide hydrolysis over time.

FIG. 5 shows cis-splicing of the AceL-TerL intein (SEQ ID NO:1). In detail, the Int^{N} and Int^{C} fragments of the AceL-TerL intein were artificially fused and inserted into a recombinant construct with MBP and FKBP as extein sequences (MBP-AceL-TerLcis-FKBP-His6). Two different linkers between the intein fragments were evaluated, a short linker of only two residues (MG) and a long linker of seven amino acids (MGSGGSG) (SEQ ID NO:7 and 8, respectively). Control constructs with mutations of two catalytically essential amino acid residues at the C-terminal splice junction (N129A, C+1A, SEQ ID NO:9 and 10, respectively) were also prepared for control experiments. All constructs were expressed in E. coli for 3 h at 28° C. following induction with IPTG. Samples were removed after 3 h and total cell lysates were used for Western Blot analysis. Calculated molecular masses are 73.5 kDa for the precursor protein (MBP-AceL-TerLcis-FKBP-His6) and 58.4 kDa for the splice product (MBP-FKBP-His6).

FIG. 6 shows cis-splicing of the selected AceL-TerL mutants in the KanR protein. Selected colonies that conferred kanamycin resistance in the selection scheme were cultivated in liquid medium supplemented with 50 μg/mL kanamycin and 100 μg/mL ampicillin at 37° C., and total cell lysates were used for Western Blot analysis (anti-His). Calculated molecular masses are 47.5 kDa for the precursor protein (His6-KanR (1-114)-AceL-TerLcis library-KanR(115-268)) and 32.4 kDa for the splice product (His6-KanR(1-114)-SGEFECEFL-KanR(115-268)). The positive control (pos.) shows the His6-Kanr protein without an intein insertion.

FIG. 7 details kinetic parameters of AceL-TerL mutant inteins with the Int^{C}-Trx-H6 constructs.

FIG. 8 details kinetic parameters of AceL-TerL mutant inteins with the MBP-Int^{C}-POI-His6 fusion proteins. Also depicted are the results obtained with the highly evolved M86 mutant of the artificially split Ssp DnaB intein, which represents the current benchmark intein for split intein-mediated N-terminal chemical modification of proteins.

FIG. 9 shows results of the characterization of the improved AceL-TerL intein mutants. In detail, time-courses of splice reactions were monitored at 37° C. and rate constants were determined by fitting product formation to pseudo-first order kinetics.

Top: Rate constants and product yields of the AceL-TerL intein (SEQ ID NO:1)
Bottom: Rate constants and product yields of the mutants M1-M6.

FIG. 10 shows more results of the characterization of the improved AceL-TerL intein mutants. In detail, rate constants of combinations of the indicated Int^{N} and Int^{C} constructs are depicted.

FIG. 11 shows further results of the characterization of the improved AceL-TerL intein mutants. In these experiments the AceL-TerL mutants were prepared as split inteins with the indicated Int^{C} fragment in the fusion constructs Int^{C}-Trx-His6 and incubated with synthetic peptides containing the Int^{N} fragment. Formation of splice and cleavage products was determined from densitometric analyses of Coomassie-stained SDS-PAGE gels. Time-courses of the splice product formation of the six mutants (native combinations of Int^{N} and Int^{C} fragments from wild-type and mutants M1-M6, SEQ ID NO:11-18) at 37° C. are shown.

FIG. 12 shows further results of the characterization of the improved AceL-TerL intein mutants. In these experiments the AceL-TerL mutants were prepared as split inteins with the indicated Int^{C} fragment in the fusion constructs Int^{C}-Trx-His6 and incubated with synthetic peptides containing the Int^{N} fragment. Formation of splice and cleavage products was determined from densitometric analyses of Coomassie-stained SDS-PAGE gels. In detail, splice product formation (top) and C-terminal cleavage product formation (bottom) of reactions combining each of the indicated Int^{C} fragments with pepWT^{N} are shown.

FIG. 13 shows further results of the characterization of the improved AceL-TerL intein mutants. In these experiments the AceL-TerL mutants were prepared as split inteins with the indicated Int^{C} fragment in the fusion constructs Int^{C}-Trx-His6 and incubated with synthetic peptides containing the Int^{N} fragment. Formation of splice and cleavage products was determined from densitometric analyses of Coomassie-stained SDS-PAGE gels. In detail, different splicing and C-terminal cleavage yields are shown. This figure illustrates, for example, that the M1 mutant has a more favourable ratio between splicing and cleavage than the M2 mutant.

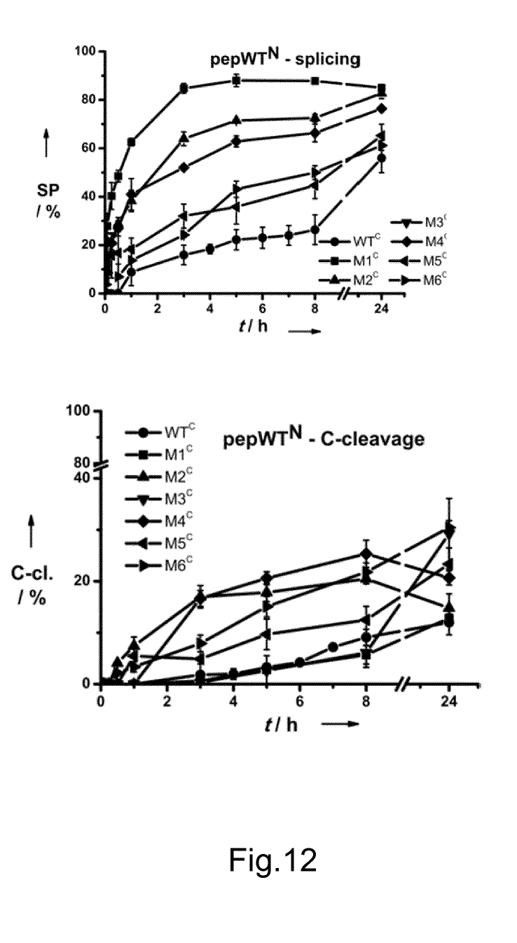

FIG. 14 depicts results demonstrating that, e.g. AceL-TerL MX1 can be generally used for protein labelling. The indicated proteins of interest (POI) were expressed and purified as fusion constructs in the format MBP-M1^{C}-POI-H6 (indicated as squares; MBP=maltose-binding protein) and incubated with pepWT^{N} at 8° C. Reactions were analyzed by SDS-PAGE using UV illumination (bottom) and Coomassie staining (top). The fluorescently labelled splice products are marked by a triangle and the MBP-M1^{C} by-products are marked by circles. Note that for each protein the lanes were normalized to the migration of the precursor protein (Abbreviations: green fluorescent protein=EGFP, red fluorescent protein=mRFP, Gaussia princeps luciferase=Gluc, murine E2 conjugating enzyme=Ubc9, human protease from the SUMO pathway=SENP1).

FIGS. 15-17

The results presented in FIGS. 15-17 also demonstrate that, e.g. AceL-TerL MX1 can be generally used for protein labelling. The AceL TerL mutant MX1 (consisting of WT-Int^{N} (SEQ ID NO:5) and M1-Int^{C} (SEQ ID NO:13) fragments) was applied in all cases. Proteins of interest (POI) were expressed and purified in the format MBP-M1^{C}-POI-His6. Precursor proteins 1-5 (15 μM) were incubated with 45 μM pepWT^{N} at 8° C., and samples were removed at the indicated time points for SDS-PAGE analysis. (EN=ExteinN sequence KKEFE).

Splicing with constructs containing the POIs

FIG. 15: eGFP and mRFP

FIG. 16: Gluc and Ubc9

FIG. 17: SENP1

FIG. 18 shows a SENP1 cleavage assay. In detail, the substrate protein SBP-HA-gpD-PML11*SUMO1 (10 μM) was incubated for 10 min at 37° C. with increasing concentrations (1 nM, 10 nM, 100 nM, 1 μM, 10 μM) of GST-SENP1 cat (positive control) or N-terminally fluorescein-labeled FI-SENP1cat (protein 10) in 20 mM HEPES, 150 mM NaCl, 1 mM DTT (pH 8). Reactions were quenched by addition of reducing SDS sample buffer, and loaded to a 15% SDS gel. Thus, this result demonstrates that the enzyme SENP1 fluorescently labeled via novel intein AceL-TerL MX1 is fully catalytically active.

FIG. 19 demonstrates the activity of the GS033_TerA-6 intein (SEQ ID NO:26). In detail, the intein construct GS033_TerA-6-Int^{C}-Trx-His6 (i.e. comprising the Int^{C} fragment with SEQ ID NO:27) was incubated with MBP-GS033_TerA-6-Int^{N}-GG-His6 (i.e. the Int^{N} fragment with SEQ ID NO:28). Aliquots were removed at indicated time points and analysed by SDS-PAGE using Coomassie Brilliant Blue staining. Positions of expected protein products are indicated.

FIG. 20 demonstrates the activity of the GS033_TerA-6 intein with a Seri Cys mutation in the Int^{N} fragment. In detail, the intein construct GS033_TerA-6-Int^{C}-Trx-His₆ (i.e. comprising the Int^{C} fragment with SEQ ID NO:27) was incubated with MBP-GS033_TerA-6-Int^{N} (S1C)-GG-His6 (i.e., the Int^{N} fragment with SEQ ID NO:29). Aliquots were removed at indicated time points and analysed by SDS-PAGE using Coomassie Brilliant Blue staining. Positions of expected protein products are indicated.

FIG. 21 demonstrates the activity of the GS033_TerA-6 intein with a truncation of 9 amino acids in the Int^{N} fragment. In detail, the intein construct GS033_TerA-6-Int^{C}-Trx-His₆ (i.e. comprising the Int^{C} fragment with SEQ ID NO:27) was incubated with MBP-GS033_TerA-6-Int^{N} (□9aa)-GG-His6 (i.e. comprising the Int^{N} fragment with SEQ ID NO:30). Aliquots were removed at indicated time points and analysed by SDS-PAGE using Coomassie Brilliant Blue staining. This experiment was repeated using a synthetic peptide comprising the Int^{N} fragment with SEQ ID NO:31 in a FI-KKEFE-Int^{N} moiety (data not shown).

FIG. 22 demonstrates the activity of the GS033_TerA-6 intein with a truncation of 3 amino acids in the Int^{N} fragment and using a synthetic peptide containing the Int^{N} fragment. In detail, the intein construct GS033_TerA-6-Int^{C}-Trx-His6 (i.e. comprising the Int^{C} fragment with SEQ ID NO:27) was incubated with FI-GS033_TerA-6-Int^{N} (□3aa) His6 (i.e. comprising the Int^{N} fragment with SEQ ID NO:32 in a FI-KKEFE-Int^{N} (delta3aa)-A moiety). Aliquots were removed at indicated time points and analysed by SDS-PAGE using UV illumination (top) and Coomassie Brilliant Blue staining (bottom). Positions of expected protein products are indicated.

FIG. 23 demonstrates the ability of the Int^{C} fragment of GS033_TerA-6 intein to trans-splice with the Int^{N} fragment of the AceL-TerL intein (cross-splicing) (i.e. the Int^{N} fragment with SEQ ID NO:5). In detail, the intein construct GS033_TerA-6-Int^{C}-Trx-His₆ (i.e. comprising the Int^{C} fragment with SEQ ID NO:27) was incubated with MBP-AceL-TerL-Int^{N}-MGGY-H₅ (ie. comprising the Int^{N} fragment with SEQ ID NO:5). Aliquots were removed at indicated time points and analysed by SDS-PAGE using Coomassie Brilliant Blue staining. Positions of expected protein products are indicated.

FIG. 24 demonstrates the ability of the Int^{C} fragment of GS033_TerA-6 intein to trans-splice with the Int^{N} fragment of the AceL-TerL intein (cross-splicing) containing an C1S amino acid substitution of the catalytic first amino acid of the intein and a Y3S mutation. In detail, the intein construct GS033_TerA-6-Int^{C}-Trx-His6 (i.e. comprising the Int^{C} fragment with SEQ ID NO:27) was incubated with FI-KKEFE-AceL-TerL-Int^{N} (C1S, Y3S) (i.e. comprising the Int^{N} fragment with SEQ ID NO:33) a construct having altered flanking residues when compared to the wild type sequence. Aliquots were removed at indicated time points and analysed by SDS-PAGE using UV illumination (data not shown) and Coomassie Brilliant Blue staining. Positions of expected protein products are indicated.

FIG. 25 shows an analysis of samples containing a mixture of the Int^{C} fragment as encoded by an expression vector coding for the construct SBP-(VidaL_T4Lh-1)^{C}-Trx-His₆ and expressed and purified from E. coli and the corresponding Int^{N} fragment of the intein as synthesized by solid-phase peptide synthesis with N-terminal 5,6-Carboxyfluoresceine (concentrations for Int^{N}-construct 9 μM, for Int^{C}-fragment 9 μM) in splice buffer (50 mM Tris/HCl, 300 mM NaCl, 1 mM EDTA, pH 7.0) with 2 mM TCEP (tris-carboxyethylphosphine) and after incubation at 25° C., quenching by mixing with SDS PAGE sample buffer and boiling at 95° C. for 5 min on a Coomassie-stained SDS PAGE gel (Mw=molecular weight marker). Before staining, the gel was also photographed under UV illumination, which revealed for fluorescently labeled band of the splice product (lower panel in FIG. 25). Formation of the expected new protein bands demonstrated the activity of the intein in semisynthetic protein trans-splicing.

FIG. 26 shows a Coomassie-stained SDS PAGE gel, in which the expression of the individual (VidaL_T4Lh-1)^{C}-Trx-His₆ construct, the expression of the individual MBP-(VidaL_T4Lh-1)^{N}-linker-SBP construct (SBP=streptavidin binding peptide), and the co-expression of both constructs is shown (from left to right; Mw=molecular weight marker). The new band appearing at 57.3 kDa is the splice product MBP-Trx-His₆. The two lanes labeled with (1) and (2) show the purified splice product after an amylose column (1) and a Ni-NTA column (2).

FIG. 27 shows an analysis by mass spectrometry of the protein sample shown in lane (2) of FIG. 26. The results further confirmed the identity of the splice product MBP-Trx-His₆ (all masses shown all average masses).

FIG. 28 shows an analysis of samples on a Coomassie-stained SDS PAGE gel (*=protein contamination; Mw=molecular weight marker). The samples were prepared as follows: The Int^{C} encoding fragment was cloned into an expression vector coding for the construct (VidaL_UvsX-2)^{C}-Trx-His₆ (Trx=thioredoxin, His₆=hexahistidine tag). The protein was produced by overexpression in E. coli and purified using Ni-NTA-chromatography. The Int^{N} fragment of the intein was synthesized by solid-phase peptide synthesis with N-terminal 5,6-Carboxyfluoresceine. Following mixing of both fragments (concentrations for Int^{N}-construct 15 μM, for Int^{C}-fragment 15 μM) in splice buffer (50 mM Tris/HCl, 300 mM NaCl, 1 mM EDTA, pH 7.0) with 2 mM TCEP (tris-carboxyethylphosphine) incubation was carried out at 25° C. Aliquots were removed at the indicated time points and quenched by mixing with SDS PAGE sample buffer and boiling at 95° C. for 5 min. Formation of the expected new protein bands demonstrated the activity of the intein in semisynthetic protein trans-splicing.

FIG. 29 shows an analysis by mass spectrometry of the samples shown in FIG. 28 confirming the molecular mass of the splice product FI-Trx-H6 (average masses are given).

### DETAILED DESCRIPTION

As stated above, the present invention is based on the unexpected finding of novel naturally split inteins that split after 14-60 amino acids from the intein's N-terminal position. “N-terminal position”, as used in this context, refers to the numbering starting from the utmost N-terminal amino acid, which is assigned position number 1. These inteins provide the shortest naturally occurring N-terminal intein fragments discovered so far. Moreover, they exhibit excellent splicing yields and rates.

In a first aspect the present invention thus relates to an isolated polypeptide comprising at least one intein or at least one fragment of said intein, wherein said intein is a naturally split intein with a N-terminal intein fragment split after 14-60 amino acids from the intein's N-terminal end.

As used herein, the term “isolated polypeptide” refers to a polypeptide, peptide or protein segment or fragment, which has been separated from other cellular components with which it may naturally associate and, in certain embodiments, which has been excised out of sequences, which flank it in a naturally occurring state. In other words, the isolated polypeptide may be a polypeptide fragment, which has been excised from a longer polypeptide sequences, in particular sequences which are normally adjacent to the fragment in the naturally occurring protein. As such, the isolated polypeptides may be artificial polypeptides. As mentioned above, the term is also used here to designate a polypeptide, which has been substantially purified from other components, which naturally accompany the polypeptide, e.g., proteins, RNA or DNA which naturally accompany it in the cell. The term therefore includes, for example, a recombinant polypeptide, which is encoded by a nucleic acid incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant polypeptide, which is part of a hybrid polypeptide comprising additional amino acids.

Moreover, the isolated polypeptide described herein or the nucleic acid encoding it may comprise in addition to all features described below regulatory sequences, i.e. segments that on nucleic acid level are capable of increasing or decreasing the expression of specific genes within an organism or segments that on protein level regulate posttranslational processing, cellular localization and the like.

The term “intein” as used herein refers to a segment of a protein capable of catalysing a protein splicing reaction that excises the intein sequence from a precursor protein and joins the flanking sequences (N- and C-exteins) with a peptide bond. Hundreds of intein and intein-like sequences have been found in a wide variety of organisms and proteins. They are typically 150-550 amino acids in size and may also contain a homing endonuclease domain.

The term “split intein” as used herein refers to any intein, in which one or more peptide bond breaks exists between the N-terminal and C-terminal amino acid sequences such that the N-terminal and C-terminal sequences become separate fragments that can non-covalently reassociate, or reconstitute, into an intein that is functional for trans-splicing reactions. In other word, a split intein, is an intein consisting of two separate polypeptides that can non-covalently associate to perform the intein function, with one of said polypeptides comprising the N-terminal part and the other comprising the C-terminal part. In case the respective polypeptides are coupled to exteins, these exteins are covalently linked by said association of the intein parts.

The term “intein fragment” as used herein refers to a separate molecule resulting from peptide bond breaks between the N-terminal and C-terminal amino acid sequences in (split) inteins. In other words, the term “intein fragment”, as used herein, relates to one of the separate parts of a split intein, in particular either the N-terminal or the C-terminal part. Such a fragment can associate with its counterpart fragment to form the active split integrin.

As the N-terminal intein fragments of the inteins described herein are comparably short, the isolated polypeptides are ideally suited for use over a wide range of protein modification techniques, such as modification of therapeutic proteins, since the protein of interest-Int^{N} (POI-Int^{N}) peptide complex or, more generally, the modifying moiety-Int^{N} peptide complex can be easily obtained using solid-phase peptide synthesis and, optionally, synthetic chemistry. Moreover, since these inteins are natural split inteins generated by evolution, they exhibit high splicing yields and rates, without exhibiting the problems encountered with split inteins artificially engineered to have short Int^{N} fragments.

In a preferred embodiment of this aspect of the present invention the N-terminal intein fragment is split after 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 amino acids as calculated from the intein's N-terminal end. In an especially preferred embodiment the N-terminal intein fragment is split after 24, 25 or 36 amino acids from the intein's N-terminal end.

In various embodiments of this aspect of the present invention the intein is a naturally split intein with a N-terminal intein fragment split after 24-37 amino acids from the intein's N-terminal position. Thus, the N-terminal intein fragment of such an intein and/or the protein of interest-Int^{N} peptide complex is even shorter and hence better suited for the chemical synthesis, e.g. for solid peptide synthesis, which is faster, easier to perform and much more reliable than protein generation via recombinant protein expression.

In a further aspect the present invention relates to an isolated polypeptide as described above, wherein said at least one intein fragment, is selected from the group comprising:
a) an N-terminal intein fragment having at least 70%, at least 80, at least 85%, at least 90%, at least 95% or 100% sequence identity with the amino acid sequence set forth in any one of SEQ ID Nos. 2, 3, 4, 5, 28, 38, 44, 50, 56, 62, 68, 74, 80, 86, 92, 98, 104, 110, 116, 122, 128, 134, 140, 146, 152, 158, 164, 170, 176 and 182, and/or
b) a C-terminal intein fragment having at least 70%, at least 80, at least 85%, at least 90%, at least 95% or 100% sequence identity with the amino acid sequence set forth in any one of SEQ ID Nos. 6, 27, 39, 45, 51, 57, 63, 69, 75, 81, 87, 93, 99, 105, 111, 117, 123, 129, 135, 141, 147, 153, 159, 165, 171, 177, 183, 194, 195 and 196, or
c) an intein having at least 70%, at least 80%, at least 85%, at least 90%, at least 95% or 100% sequence identity with the amino acid sequence set forth in SEQ ID NO:1 or SEQ ID NO:26.

In a preferred embodiment of the invention, the split intein is formed by two separate polypeptides that non-covalently associate, i.e. there is one C-terminal intein fragment and one N-terminal intein fragment.

As interchangeably used herein, the terms “N-terminal split intein, “N-terminal intein fragment” and “N-terminal intein sequence” (abbreviated “Int^{N}”)” refer to any intein sequence that comprises an N-terminal amino acid sequence that is functional for trans-splicing reactions. It thus also comprises a sequence that is spliced out when trans-splicing occurs. It can comprise a sequence that is a modification of the N-terminal portion of a naturally occurring intein sequence. For example, it can comprise additional amino acid residues and/or mutated residues so long as the inclusion of such additional and/or mutated residues does not render the Int^{N} non-functional in trans-splicing. Preferably, the inclusion of the additional and/or mutated residues improves or enhances the trans-splicing activity of the Int^{N}.

As interchangeably used herein, the terms “C-terminal split intein”, “C-terminal intein fragment” and “C-terminal intein sequence” (abbreviated “Int^{C}”)” refer to any intein sequence that comprises a C-terminal amino acid sequence that is functional for trans-splicing reactions. An Int^{C} thus also comprises a sequence that is spliced out when trans-splicing occurs. An Int^{C} can comprise a sequence that is a modification of the C-terminal portion of a naturally occurring intein sequence. For example, it can comprise additional amino acid residues and/or mutated residues so long as the inclusion of such additional and/or mutated residues does not render the Int^{C} non-functional in trans-splicing. Preferably, the inclusion of the additional and/or mutated residues improves or enhances the trans-splicing activity of the Int^{C}.

The term “sequence identity” as used herein refers to peptides that share identical amino acids at corresponding positions or nucleic acids sharing identical nucleotides at corresponding positions. In order to take into account the fact that peptides may exist which do not have significant “sequence identity”, as they may not have similar amino acids at corresponding positions, but have the same function, because they contain, e.g., conservative substitutions, the amino acid sequences herein are referred to in the context of percent identity.

The determination of percent identity described herein between two amino acid or nucleotide sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs and can be accessed, for example, at the National Center for Biotechnology Information (NCBI) world wide web site having the universal resource locator “www.ncbi.nlm.nih.gov/BLAST”. Blast nucleotide searches can be performed with BLASTN program, whereas BLAST protein searches can be performed with BLASTX program or the NCBI “blastp” program.

The term “mutant” as used herein refers to polypeptide the sequence of which has one or more amino acids added, deleted, substituted or otherwise chemically modified in comparison to a reference polypeptide, for example one of the claimed sequences, provided that the mutant retains substantially the same properties as the reference polypeptide. “Substantially the same properties”, in various embodiments, relates to the fact that a given mutant has at least 50%, preferably at least 75% or more of the activity of the reference polypeptide.

In various embodiments, the isolated polypeptide comprises at least an N-terminal intein fragment having at least 70%, or 80% or 85% or 90% or 95% or 100% sequence identity with the amino acid sequence set forth in any one of SEQ ID Nos. 2, 3, 4, 5, 28, 38, 44, 50, 56, 62, 68, 74, 80, 86, 92, 98, 104, 110, 116, 122, 128, 134, 140, 146, 152, 158, 164, 170, 176 and 182.

In various embodiments, the isolated polypeptide comprises at least a C-terminal intein fragment having at least 70%, or 80% or 85% or 90% or 95% or 100% sequence identity with the amino acid sequence set forth in any one of SEQ ID Nos. 6, 27, 39, 45, 51, 57, 63, 69, 75, 81, 87, 93, 99, 105, 111, 117, 123, 129, 135, 141, 147, 153, 159, 165, 171, 177, 183, 194, 195 or 196.

To form the functional split intein, the polypeptide comprising at least one N-terminal intein fragment as defined above and the polypeptide comprising at least one C-terminal intein fragment may be combined. It is understood that the functional split intein is formed, in various embodiments, by two of the isolated polypeptides described herein, one comprising the N-terminal and the other the C-terminal part, with both being separate molecules, i.e. not being covalently linked by a peptide bond.

The isolated polypeptides described herein can advantageously be used for example for labelling of a protein. Due to the small size of the N-terminal intein fragment the protein of interest-Int^{N} (POI-Int^{N}) peptide complex can be obtained by using solid-phase peptide synthesis. The label e.g. EGFP attached to the Int^{C} fragment (Int^{C}-EGFP) can be generated by recombinant protein expression. Upon combining the two chimeric protein complexes, i.e. POI-Int^{N} and Int^{C}-EGFP, the trans splicing reaction could take place generating POI-EGFP. Of course, also encompassed are all embodiments wherein N- and C-terminal intein fragments are exchanged, i.e. by coupling the label or any other modifying moiety (that need not be a peptide or protein but only needs to be coupled to an amino acid or amino acid oligomer) to the N-terminal intein fragment and synthesizing the protein of interest as a recombinant fusion protein with the C-terminal intein fragment. It is thus understood that while embodiments may be described herein with reference to only one of these possibilities the present invention is intended to also cover the respective counterpart where the two intein fragments are exchanged.

In various embodiments, the two separate intein fragments are useful by themselves. For example, it is possible, to pre-assemble—possibly in form of a kit—the Int^{C}-EGFP fusion protein or merely the Int^{C} fragment, e.g., for easy protein labelling. The ready Int^{C}-EGFP fusion proteins could be then used as soon as a protein of interest is decided upon for easy and robust protein labelling. Of course, the reverse scenario is also possible, where the protein of interest is known and pre-generated fused to the Int^{N} fragment. As soon as it is decided upon which labels should be used the Int^{C}-label fusion proteins could be prepared and protein labelling could be carried out.

Moreover, the EGFP of the fusion protein in this example can of course be readily replaced by any protein of interest or any other non-peptide, non-protein moiety. In case non-peptide, non-protein moieties are used for modification of proteins or any other purpose, these are used in form of conjugates with at least one amino acid or a short peptide sequence to facilitate the covalent linkage with the corresponding other extein part by a peptide bond.

In various embodiments, including the afore-mentioned, it is preferred that the Int^{C}-protein fusion protein, or more generally the intein fragment-protein of interest fusion protein, is generated via recombinant expression.

In various embodiments of this aspect of the invention, one of the intein fragments can be attached to a short PEG linker with a thiol group and then bound to (immobilized on) a maleimido-coated glass surface. Upon addition of the protein of interest fused to the complementary intein fragment and trans-splicing the protein of interest would remain bound to the glass surface. Thus, it is possible to preassemble such a glass surface, e.g. with the Int^{N} fragment, in order to later on immobilize any protein of interest fused to the complementary Int^{C} fragment. Moreover, Int^{N} fragment preassembled in such a fashion could act as capture probe array.

In various embodiments of this aspect of the present invention an isolated polypeptide comprises at least one N-terminal intein fragment and at least one C-terminal intein fragment, wherein:
1) the at least one N-terminal intein fragment is selected from SEQ ID NO:5 and the at least one C-terminal intein fragment is selected from SEQ ID N 6, or
2) the at least one N-terminal intein fragment is selected from SEQ ID NO:5, 28-33 and the at least one C-terminal intein fragment is selected from SEQ ID NO:27, or
3) the at least one N-terminal intein fragment is selected from SEQ ID NO:38 and the at least one C-terminal intein fragment is selected from SEQ ID NO:39, or
4) the at least one N-terminal intein fragment is selected from SEQ ID NO:44 and the at least one C-terminal intein fragment is selected from SEQ ID NO:45, or
5) the at least one N-terminal intein fragment is selected from SEQ ID NO:50 and the at least one C-terminal intein fragment is selected from SEQ ID NO:51, or
6) the at least one N-terminal intein fragment is selected from SEQ ID NO:56 and the at least one C-terminal intein fragment is selected from SEQ ID NO:57, or
7) the at least one N-terminal intein fragment is selected from SEQ ID NO:62 and the at least one C-terminal intein fragment is selected from SEQ ID NO:63, or
8) the at least one N-terminal intein fragment is selected from SEQ ID NO:68 and the at least one C-terminal intein fragment is selected from SEQ ID NO:69, or
9) the at least one N-terminal intein fragment is selected from SEQ ID NO:74 and the at least one C-terminal intein fragment is selected from SEQ ID NO:75, or
10) the at least one N-terminal intein fragment is selected from SEQ ID NO:80 and the at least one C-terminal intein fragment is selected from SEQ ID NO:81, or
11) the at least one N-terminal intein fragment is selected from SEQ ID NO:86 and the at least one C-terminal intein fragment is selected from SEQ ID NO:87, or
12) the at least one N-terminal intein fragment is selected from SEQ ID NO:92 and the at least one C-terminal intein fragment is selected from SEQ ID NO:93, or
13) the at least one N-terminal intein fragment is selected from SEQ ID NO:98 and the at least one C-terminal intein fragment is selected from SEQ ID NO:99, or
14) the at least one N-terminal intein fragment is selected from SEQ ID NO:104 and the at least one C-terminal intein fragment is selected from SEQ ID NO:105, or
15) the at least one N-terminal intein fragment is selected from SEQ ID NO:110 and the at least one C-terminal intein fragment is selected from SEQ ID NO:111, or
16) the at least one N-terminal intein fragment is selected from SEQ ID NO:116 and the at least one C-terminal intein fragment is selected from SEQ ID NO:117, or
17) the at least one N-terminal intein fragment is selected from SEQ ID NO:122 and the at least one C-terminal intein fragment is selected from SEQ ID NO:123, or
18) the at least one N-terminal intein fragment is selected from SEQ ID NO:128 and the at least one C-terminal intein fragment is selected from SEQ ID NO:129, or
19) the at least one N-terminal intein fragment is selected from SEQ ID NO:134 and the at least one C-terminal intein fragment is selected from SEQ ID NO:135, or
20) the at least one N-terminal intein fragment is selected from SEQ ID NO:140 and the at least one C-terminal intein fragment is selected from SEQ ID NO:141, or
21) the at least one N-terminal intein fragment is selected from SEQ ID NO:146 and the at least one C-terminal intein fragment is selected from SEQ ID NO:147, or
22) the at least one N-terminal intein fragment is selected from SEQ ID NO:152 and the at least one C-terminal intein fragment is selected from SEQ ID NO:153, or
23) the at least one N-terminal intein fragment is selected from SEQ ID NO:158 and the at least one C-terminal intein fragment is selected from SEQ ID NO:159, or
24) the at least one N-terminal intein fragment is selected from SEQ ID NO:164 and the at least one C-terminal intein fragment is selected from SEQ ID NO:165, or
25) the at least one N-terminal intein fragment is selected from SEQ ID NO:170 and the at least one C-terminal intein fragment is selected from SEQ ID NO:171, or
26) the at least one N-terminal intein fragment is selected from SEQ ID NO:176 and the at least one C-terminal intein fragment is selected from SEQ ID NO:177, or
27) the at least one N-terminal intein fragment is selected from SEQ ID NO:182 and the at least one C-terminal intein fragment is selected from SEQ ID NO:183, or
28) the at least one N-terminal intein fragment is selected from SEQ ID NO:2 and the at least one C-terminal intein fragment is selected from SEQ ID NO:194, or
29) the at least one N-terminal intein fragment is selected from SEQ ID NO:3 and the at least one C-terminal intein fragment is selected from SEQ ID NO:195, or
30) the at least one N-terminal intein fragment is selected from SEQ ID NO:4 and the at least one C-terminal intein fragment is selected from SEQ ID NO:196.

In these embodiments, the two fragments that naturally occur in form of separate molecules may be combined in one molecule. Alternatively, the two fragments may still be parts of separate molecules. In the latter case, the isolated polypeptide is a combination of at least two, preferably two, isolated polypeptides, one of which comprises the N-terminal intein fragment, as defined above, and the other comprising the C-terminal intein fragment, also as defined above. The present invention therefore also covers combinations of two isolated polypeptides as described herein, wherein an isolated polypeptide comprises at least one N-terminal intein fragment and at least one C-terminal intein fragment, wherein the first polypeptide comprises at least one N-terminal intein fragment and the second polypeptide comprises at least one C-terminal intein fragment, wherein:
1) the at least one N-terminal intein fragment is selected from SEQ ID NO:5 and the at least one C-terminal intein fragment is selected from SEQ ID N 6, or
2) the at least one N-terminal intein fragment is selected from SEQ ID NO:5, 28-33 and the at least one C-terminal intein fragment is selected from SEQ ID NO:27, or
3) the at least one N-terminal intein fragment is selected from SEQ ID NO:38 and the at least one C-terminal intein fragment is selected from SEQ ID NO:39, or
4) the at least one N-terminal intein fragment is selected from SEQ ID NO:44 and the at least one C-terminal intein fragment is selected from SEQ ID NO:45, or
5) the at least one N-terminal intein fragment is selected from SEQ ID NO:50 and the at least one C-terminal intein fragment is selected from SEQ ID NO:51, or
6) the at least one N-terminal intein fragment is selected from SEQ ID NO:56 and the at least one C-terminal intein fragment is selected from SEQ ID NO:57, or
7) the at least one N-terminal intein fragment is selected from SEQ ID NO:62 and the at least one C-terminal intein fragment is selected from SEQ ID NO:63, or
8) the at least one N-terminal intein fragment is selected from SEQ ID NO:68 and the at least one C-terminal intein fragment is selected from SEQ ID NO:69, or
9) the at least one N-terminal intein fragment is selected from SEQ ID NO:74 and the at least one C-terminal intein fragment is selected from SEQ ID NO:75, or
10) the at least one N-terminal intein fragment is selected from SEQ ID NO:80 and the at least one C-terminal intein fragment is selected from SEQ ID NO:81, or
11) the at least one N-terminal intein fragment is selected from SEQ ID NO:86 and the at least one C-terminal intein fragment is selected from SEQ ID NO:87, or
12) the at least one N-terminal intein fragment is selected from SEQ ID NO:92 and the at least one C-terminal intein fragment is selected from SEQ ID NO:93, or
13) the at least one N-terminal intein fragment is selected from SEQ ID NO:98 and the at least one C-terminal intein fragment is selected from SEQ ID NO:99, or
14) the at least one N-terminal intein fragment is selected from SEQ ID NO:104 and the at least one C-terminal intein fragment is selected from SEQ ID NO:105, or
15) the at least one N-terminal intein fragment is selected from SEQ ID NO:110 and the at least one C-terminal intein fragment is selected from SEQ ID NO:111, or
16) the at least one N-terminal intein fragment is selected from SEQ ID NO:116 and the at least one C-terminal intein fragment is selected from SEQ ID NO:117, or
17) the at least one N-terminal intein fragment is selected from SEQ ID NO:122 and the at least one C-terminal intein fragment is selected from SEQ ID NO:123, or
18) the at least one N-terminal intein fragment is selected from SEQ ID NO:128 and the at least one C-terminal intein fragment is selected from SEQ ID NO:129, or
19) the at least one N-terminal intein fragment is selected from SEQ ID NO:134 and the at least one C-terminal intein fragment is selected from SEQ ID NO:135, or
20) the at least one N-terminal intein fragment is selected from SEQ ID NO:140 and the at least one C-terminal intein fragment is selected from SEQ ID NO:141, or
21) the at least one N-terminal intein fragment is selected from SEQ ID NO:146 and the at least one C-terminal intein fragment is selected from SEQ ID NO:147, or
22) the at least one N-terminal intein fragment is selected from SEQ ID NO:152 and the at least one C-terminal intein fragment is selected from SEQ ID NO:153, or
23) the at least one N-terminal intein fragment is selected from SEQ ID NO:158 and the at least one C-terminal intein fragment is selected from SEQ ID NO:159, or
24) the at least one N-terminal intein fragment is selected from SEQ ID NO:164 and the at least one C-terminal intein fragment is selected from SEQ ID NO:165, or
25) the at least one N-terminal intein fragment is selected from SEQ ID NO:170 and the at least one C-terminal intein fragment is selected from SEQ ID NO:171, or
26) the at least one N-terminal intein fragment is selected from SEQ ID NO:176 and the at least one C-terminal intein fragment is selected from SEQ ID NO:177, or
27) the at least one N-terminal intein fragment is selected from SEQ ID NO:182 and the at least one C-terminal intein fragment is selected from SEQ ID NO:183, or
28) the at least one N-terminal intein fragment is selected from SEQ ID NO:2 and the at least one C-terminal intein fragment is selected from SEQ ID NO:194, or
29) the at least one N-terminal intein fragment is selected from SEQ ID NO:3 and the at least one C-terminal intein fragment is selected from SEQ ID NO:195, or
30) the at least one N-terminal intein fragment is selected from SEQ ID NO:4 and the at least one C-terminal intein fragment is selected from SEQ ID NO:196.

Advantageously the resulting polypeptide has split intein activity exhibiting excellent splicing yields and rates.

Furthermore, of course all application envisaged for one of the intein fragments also apply for both together.

In preferred embodiments of this aspect of the present invention the isolated polypeptide comprises exactly one N-terminal intein fragment and exactly one C-terminal intein fragment selected as described above. This similarly applies in case two separate isolated polypeptides are used.

In yet another aspect the present invention relates to an isolated polypeptide as described above, wherein said polypeptide at the N-terminal end of the at least one N-terminal intein fragment and/or at the C-terminal end of the at least one C-terminal intein fragment further comprises a flanking amino acid sequence, wherein said flanking amino acid sequence is selected from:
1) in the case of SEQ ID NO:5 and/or 6 SEQ ID NO:25 and/or 26, respectively
2) in the case of SEQ ID NO:5, 28-33 and/or 27 SEQ ID NO:34 and/or 35, respectively
3) in the case of SEQ ID NO:38 and/or 39 SEQ ID NO:40 and/or 41, respectively
4) in the case of SEQ ID NO:44 and/or 45 SEQ ID NO:46 and/or 47, respectively
5) in the case of SEQ ID NO:50 and/or 51 SEQ ID NO:52 and/or 53, respectively
6) in the case of SEQ ID NO:56 and/or 57 SEQ ID NO:58 and/or 59, respectively
7) in the case of SEQ ID NO:62 and/or 63 SEQ ID NO:64 and/or 65, respectively
8) in the case of SEQ ID NO:68 and/or 69 SEQ ID NO:70 and/or 71, respectively
9) in the case of SEQ ID NO:74 and/or 75 SEQ ID NO:76 and/or 77, respectively
10) in the case of SEQ ID NO:80 and/or 81 SEQ ID NO:82 and/or 83, respectively
11) in the case of SEQ ID NO:86 and/or 87 SEQ ID NO:88 and/or 89, respectively
12) in the case of SEQ ID NO:92 and/or 93 SEQ ID NO:94 and/or 95, respectively
13) in the case of SEQ ID NO:98 and/or 99 SEQ ID NO:100 and/or 101, respectively
14) in the case of SEQ ID NO:104 and/or 105 SEQ ID NO:106 and/or 107, respectively
15) in the case of SEQ ID NO:110 and/or 111 SEQ ID NO:112 and/or 113, respectively
16) in the case of SEQ ID NO:116 and/or 117 SEQ ID NO:118 and/or 119, respectively
17) in the case of SEQ ID NO:122 and/or 123 SEQ ID NO:124 and/or 125, respectively
18) in the case of SEQ ID NO:128 and/or 129 SEQ ID NO:130 and/or 131, respectively
19) in the case of SEQ ID NO:134 and/or 135 SEQ ID NO:136 and/or 137, respectively
20) in the case of SEQ ID NO:140 and/or 141 SEQ ID NO:142 and/or 143, respectively
21) in the case of SEQ ID NO:146 and/or 147 SEQ ID NO:148 and/or 149, respectively
22) in the case of SEQ ID NO:152 and/or 153 SEQ ID NO:154 and/or 155, respectively
23) in the case of SEQ ID NO:158 and/or 159 SEQ ID NO:160 and/or 161, respectively
24) in the case of SEQ ID NO:164 and/or 165 SEQ ID NO:166 and/or 167, respectively
25) in the case of SEQ ID NO:170 and/or 171 SEQ ID NO:172 and/or 173, respectively
26) in the case of SEQ ID NO:176 and/or 177 SEQ ID NO:178 and/or 179, respectively
27) in the case of SEQ ID NO:182 and/or 183 SEQ ID NO:184 and/or 185, respectively.

In various embodiments, the above is to be understood such that, for example, the N-terminal intein fragment of SEQ ID NO:5 is N-terminally flanked by SEQ ID NO: 25, i.e. SEQ ID NO:25 is located N-terminal to SEQ ID NO:5, and the C-terminal fragment of SEQ ID NO:6 is C-terminally flanked by SEQ ID NO:26, i.e. SEQ ID NO:26 is located C-terminal to SEQ ID NO:6.

Such an isolated polypeptide has the advantage that the autocatalytic reaction—the protein splicing—proceeds with higher efficiency, if 1-5 of the wild type extein residues (also termed flanking sequences, i.e. sequences flanking the intein) are present.

If the intein fragments are part of different polypeptides, the respective flanking sequences may be comprised in the respective polypeptide.

In this context, it is emphasized that in general the sequences in this application are shown without the +1 residue following the Int^{C}, as this residue is strictly not part of the intein. However, it should be noted that this residue is usually involved in the intein's activity and forms part of the intein active site.

In case of an isolated polypeptide/isolated polypeptides comprising one N-terminal intein fragment and one C-terminal intein fragment, wherein the N-terminal intein fragment has at least 70%, or 80% or 85% or 90% or 95% or 100% sequence identity with SEQ ID NO:5 and the C-terminal intein fragment has at least 70%, or 80% or 85% or 90% or 95% or 100% sequence identity with SEQ ID NO:6, the resulting intein has an activity maximum at low temperatures such as 8° C. This is ideal to preserve potentially fragile proteins of interest for example in in vitro protein labelling experiments.

Moreover, it was surprisingly possible to further modify the AceL-TerL intein (SEQ ID NO:1) to increase splicing yields and rates even at 37° C.

Thus, in a further aspect the present invention relates to an isolated polypeptide wherein the N-terminal intein fragment has 100% sequence identity with a sequence selected from sequences comprising SEQ ID NO:5, 11 and 12, or has at least 90% or 95% sequence identity with a sequence selected from sequences comprising SEQ ID NO:5, 11 or 12 and/or, wherein the C-terminal intein fragment has 100% sequence identity with a sequence selected from sequences comprising SEQ ID NO:6 or 13-18 or has at least 90% or 95% sequence identity with a sequence selected from sequences comprising SEQ ID NO:6 or 13-18.

Such isolated polypeptides provide novel split inteins ideally suited for protein modification and semi-synthesis due to their superior splicing yields and rates.

In various embodiments of this aspect of the invention the isolated polypeptide comprises at least one N-terminal intein fragment and at least one C-terminal intein fragment wherein the N-terminal intein fragment is selected from sequences comprising SEQ ID NO:5, 11 or 12 and the C-terminal intein fragment is selected from sequences comprising SEQ ID NO:6 or 13-18. Alternatively, the N-terminal intein fragment and the C-terminal intein fragment may be part of separate isolated polypeptides.

Besides their superior splicing yields and rates the novel split inteins are capable of efficient splicing at 37° C. This characteristic is advantageous as it renders the inteins more thermostable, better suited for expression of fusion proteins in organisms such as E. coli and in general broadens their practical utility for a range of applications.

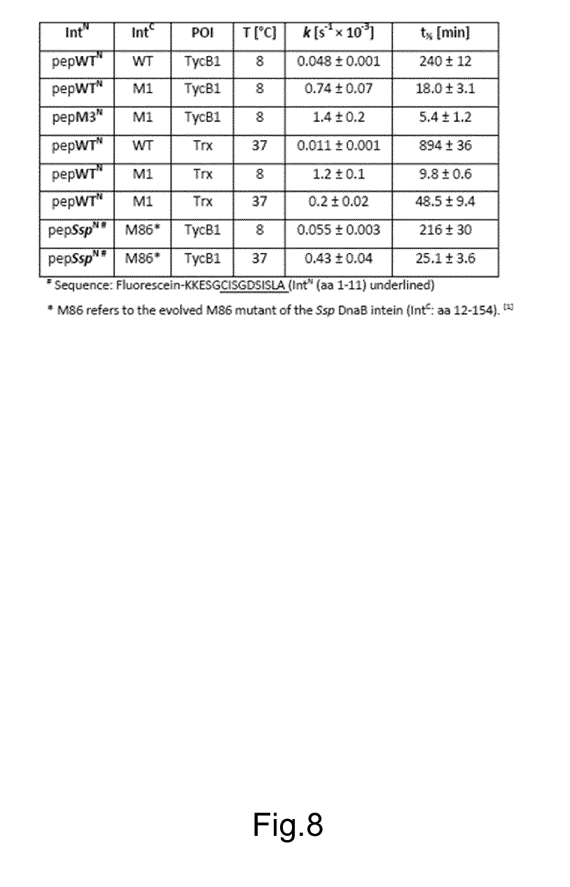

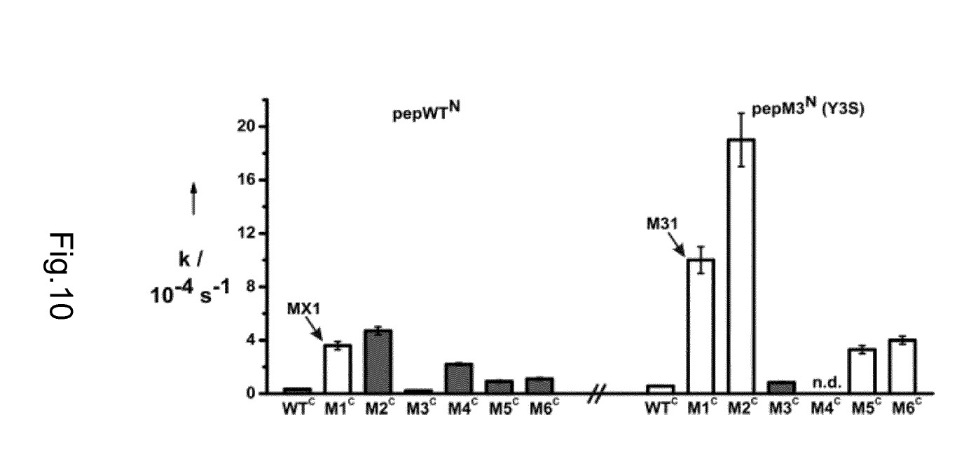

In detail, a clear improvement over the wild-type AceL-TerL intein trans-splicing reactions at 37° C. could be observed for all the mutants. Rates were increased by about 2- to 14-fold (FIG. 9 and FIG. 11) and yields were increased to 65-85% after 24 h compared to about 60% for the wild-type intein, with concomitant decrease of the C-cleavage product (FIG. 9).

Especially preferred combinations of N-terminal intein fragments selected from WT^{N}, M1^{N}, M3^{N} and C-terminal intein fragments selected from sequences WT^{C}, M1^{C}, M2^{C}, M3^{C}, M4^{C}, M5^{C}, M6^{C} are listed in Table 1.

As used herein the term “wild-type” refers to the phenotype of the typical form of a species as it occurs in nature. In relation to the provided intein sequences it should be noted that the term can also refer to artificially joined sequences, which themselves possess the wild-type succession of amino acids or nucleotides, respectively.

The term “splicing yield” as used herein refers to the amount of splice product produced. Ideally the intein mediated trans-splicing reaction would only result in splice product comprising the extein sequences attached to the intein fragments prior to the reaction. However, under unfavourable conditions or when using an intein, which is not suitable, the formation of cleavage side-product may occur. This lowers the overall splicing yield.

The term “splicing rate” as used herein refers to the change in concentration of the products per unit time. It is expressed using a rate constant k. The rate constant, k, which is temperature dependent, is a proportionality constant for a given reaction.

In still a further aspect the present invention relates to an isolated polypeptide wherein the polypeptide comprises the N-terminal intein fragment with SEQ ID NO:5 or 12 and the C-terminal intein fragment with SEQ ID NO:13. Alternatively, the N-terminal intein fragment and the C-terminal intein fragment may be part of separate isolated polypeptides.

Such an isolated polypeptide provides a novel split intein with even better splicing yields and rates.

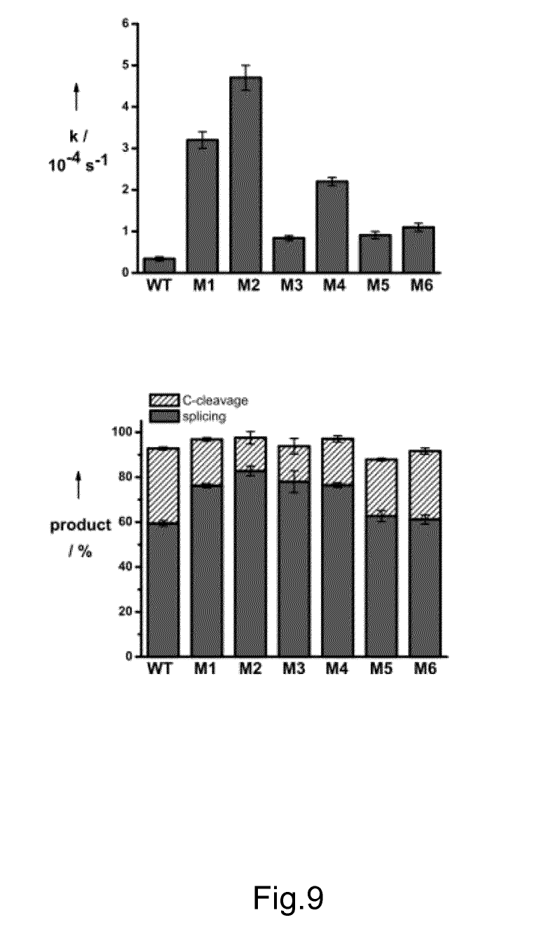

In detail, these combinations spliced significantly faster with a beneficial ratio between splicing and cleavage yields (FIG. 10).

In yet a further aspect the present invention relates to an isolated polypeptide as described above wherein the N-terminal intein fragment has 100% sequence identity with a sequence selected from sequences comprising SEQ ID NOs: 5, 28-33 or has at least 90% or 95% sequence identity with a sequence selected from sequences comprising SEQ ID NOs: 28-33 and/or, wherein the C-terminal intein fragment has 100% sequence identity with SEQ ID NO:27 or has at least 90% or 95% sequence identity with with SEQ ID NO:27. Alternatively, the N-terminal intein fragment and the C-terminal intein fragment may be part of separate isolated polypeptides.

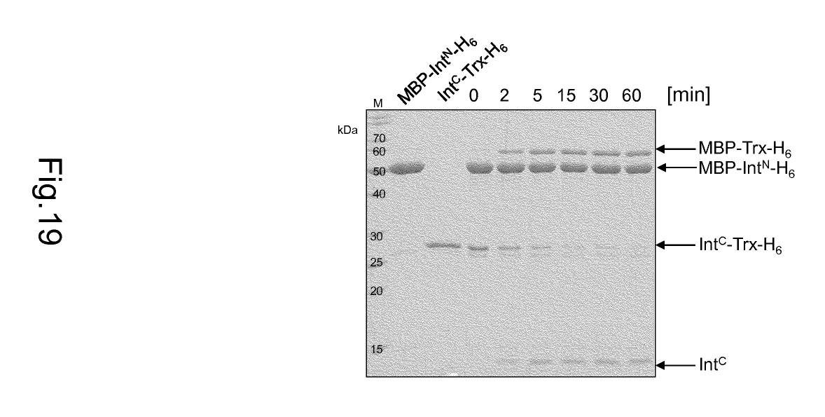

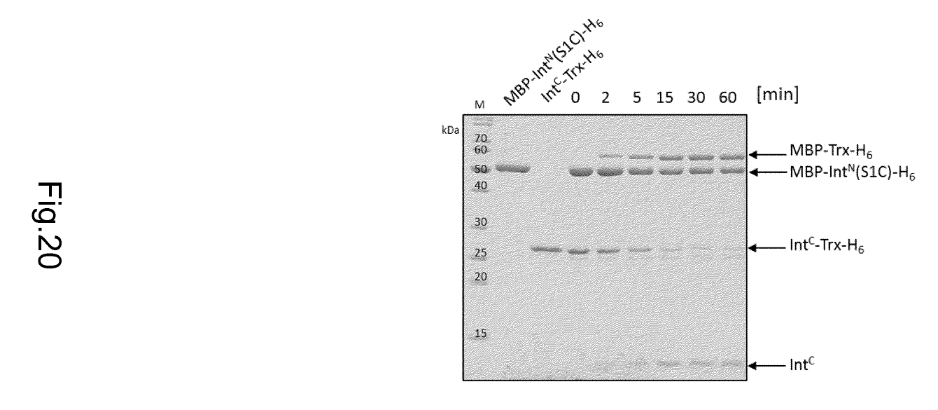

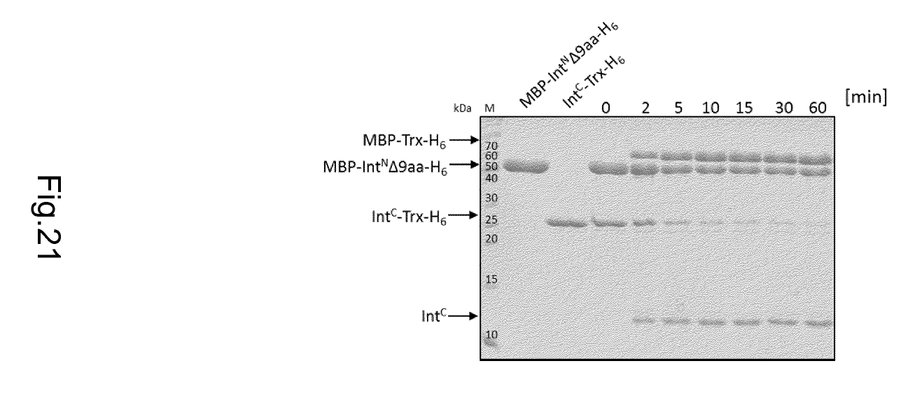

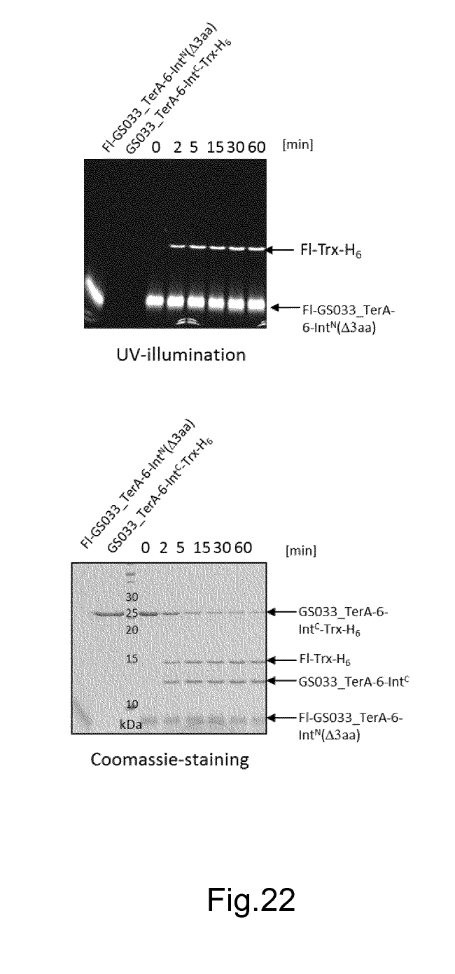

As shown in FIGS. 19-22 these polypeptides exhibit high splicing yields and rates, even if the Int^{N} and/or Int^{C} sequences were slightly modified.

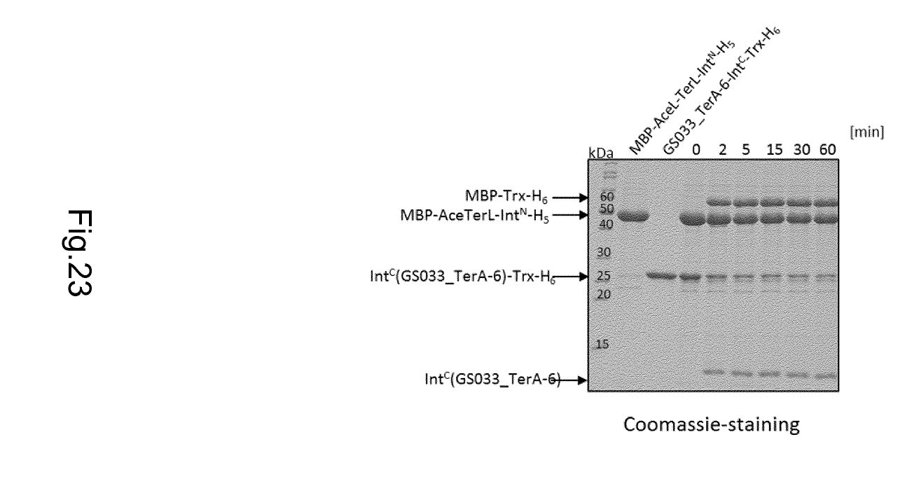

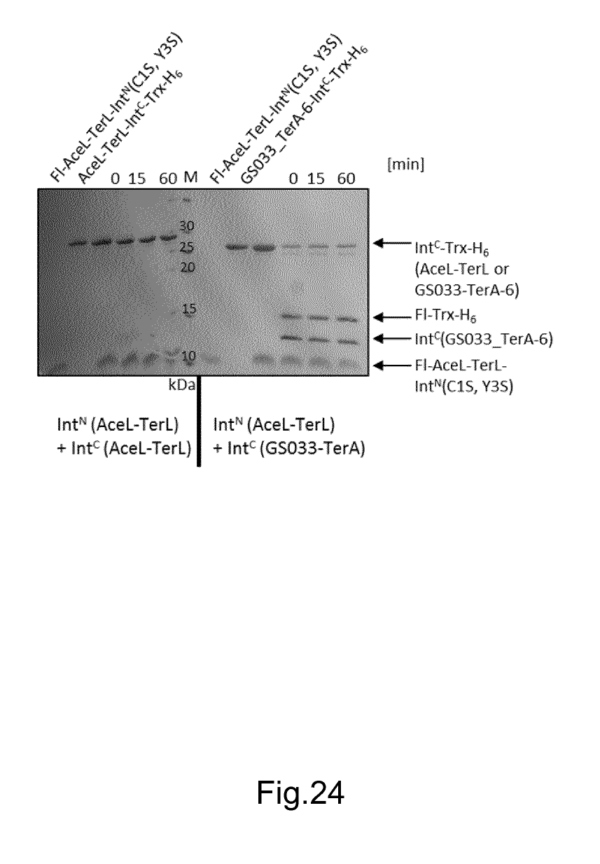

Moreover, it was unexpectedly found that the Int^{C} with SEQ ID NO:27 is not only capable of splicing with the wild-type Int^{N} sequence with SEQ ID NO:28, but is also capable of splicing with the Int^{N} with SEQ ID NO:5. In addition, this cross splicing proceeds with both the original Cys1 of SEQ ID NO:5 and with a Seri at this position (cf., FIGS. 23 and 24). The C1S substitution could be advantageous, for example in order to minimize the oxidation risk of the peptide.

In addition, the fact that the Int^{C} with SEQ ID NO:27 is capable of splicing with the Int^{N} with SEQ IS NO 5, i.e. cross-splices, is advantageous as it substantially extends the possibilities for use of both intein fragments. In other words, it could for example be envisaged to pre-assemble—possibly in form of a kit—an Int^{C}-EGFP fusion protein comprising SEQ ID NO:27 or merely the Int^{C} fragment, e.g., for easy protein labelling. The ready Int^{C}-EGFP fusion proteins could then be used with either the Int^{N} of SEQ ID NO:5, SEQ ID NO:28-33, whichever one is available, cheapest to generate or for other reasons most suitable for easy and robust protein labelling.

In still a further aspect, the present invention relates to an isolated polypeptide further comprising at least one C-terminal extein or at least one N-terminal extein sequence.

The term “extein” or “extein sequence” as used herein refers to the peptide sequences that link to form a new polypeptide after the intein has excised itself during splicing. In other words, the N-terminal and C-terminal exteins (also termed Ext^{N} and Ex^{C}) flank the Int^{N} and Int^{C} fragments, respectively prior to the trans-splicing reaction.

In various embodiments of this aspect of the present invention the isolated polypeptide comprises at least one C-terminal extein and least one N-terminal extein sequence.

In various embodiments of this aspect of the present invention the isolated polypeptide comprises exactly one C-terminal extein and exactly one N-terminal extein sequence.

In yet still other embodiments of this aspect of the present invention at least one of the peptide sequences of the isolated polypeptide selected from N-terminal intein, C-terminal intein, C-terminal extein and/or N-terminal extein is a recombinant protein.

In various embodiments, the extein sequence is heterologous with respect to the intein fragment to which it is coupled, i.e. the two sequences do not naturally occur together but have been artificially combined.

It is understood that all the above embodiments are similarly applicable to scenarios where the N-terminal intein fragment and the C-terminal intein fragment are part of separate molecules. In such cases each of the two molecules may comprise the respective extein sequence, i.e. the polypeptide comprising the N-terminal intein fragment comprises the N-terminal extein and the polypeptide comprising the C-terminal intein fragment comprises the C-terminal extein.

Through using extein sequences that are heterologous to the chosen intein sequences, the full power of split inteins can be exploited, as the extein sequences will be joined by a peptide bond following the trans-splicing reaction with virtually no trace of the previously existing intein sequences.

Thus, in a further aspect the invention relates to two isolated polypeptides as described above, for example in form of a combination or composition, wherein one of the isolated polypeptides comprises least one heterologous N-terminal extein fused to an N-terminal intein fragment having at least 70%, at least 80%, at least 85%, at least 90%, at least 95%, at least 99% or 100 sequence identity with the amino acid sequence set forth in any one of SEQ ID Nos. 2, 3, 4, 5, 28, 38, 44, 50, 56, 62, 68, 74, 80, 86, 92, 98, 104, 110, 116, 122, 128, 134, 140, 146, 152, 158, 164, 170, 176 and 182, and wherein the second one of the isolated polypeptides comprises at least one C-terminal extein sequence fused to a C-terminal intein fragment having at least 70%, at least 80%, at least 85%, at least 90%, at least 95%, at least 99% or 100 sequence identity with the amino acid sequence set forth in any one of SEQ ID Nos. 6, 27, 39, 45, 51, 57, 63, 69, 75, 81, 87, 93, 99, 105, 111, 117, 123, 129, 135, 141, 147, 153, 159, 165, 171, 177, 183, 194, 195, 196.

All embodiments disclosed above in relation to one isolated polypeptide are similarly applicable to such embodiments where two isolated polypeptides are used. This particularly applies to the above described combinations of intein fragments and their combination with flanking sequences.

Advantageously, the complementary Int^{N} and Int^{C} fragments with their respective extein sequences are used together, as thereby the full potential of split inteins is realized, e.g. in applications such as modification of a protein, protein lipidation, protein immobilization, protein backbone semisynthetic, artificial control of protein splicing by light and use as a molecular switch. This is especially the case, if the extein sequences are heterologous to the intein fragments and possibly to each other. As mentioned above in such a case the extein sequences, which can be synthesized separately, will be joined by a peptide bond following the trans-splicing reaction with virtually no trace of the previously existing intein sequences.

In various embodiments of all aspect of the present invention the isolated polypeptide is a contiguous cis splicing polypeptide.

The term “cis splicing polypeptide” as used herein refers to a polypeptide, which has a continuous polypeptide sequence.

In a preferred embodiment the contiguous cis splicing polypeptide has the amino acid sequence of SEQ ID NO:7 or 8.

In other embodiments of all aspects of the present invention, the isolated polypeptide is a trans splicing polypeptide, i.e. has a discontinuous polypeptide sequence that needs complementation by another trans splicing polypeptide to become active. In such embodiments each of the trans splicing polypeptides as such as well as the combination of both, for example in form of a non-covalently associated complex, is intended to be encompassed by the present invention.

In a further aspect the present invention relates to an isolated polypeptide further comprising at least one component selected from a solubility factor, a marker, a linker, an epitope, an affinity tag, a fluorophore or a fluorescent protein, a toxic compound or protein and a small-molecule or a small-molecule binding protein.

As used herein the term “marker” refers to a component allowing detecting directly or indirectly the molecules having said marker. Thus, in some embodiments the marker is a label such as a fluorophore.

As used herein the term “linker” refers to a chemical moiety that connects parts of the isolated polypeptide or the nucleic acids described herein. Thus, a linker may be especially employed in the case of cis splicing polypeptides.

As used herein the term “epitope” refers to an immunogenic amino acid sequence.

As used herein the term “affinity tag” refers to a polypeptide sequence, which has affinity for a specific capture reagent and which can be separated from a pool of proteins and thus purified on the basis of its affinity for the capture reagent.

As used herein the term “fluorophore” means a compound or group that fluoresces when exposed to and excited by a light source, i.e. it re-emits light.

As used herein the term “fluorescent protein” refers to a protein that is fluorescent, e.g., it may exhibit low, medium or intense fluorescence upon exposure to electromagnetic radiation.

As used herein the term “small molecule” refers to any molecule, or chemical entity, with a molecular weight of less than about 1,000 Daltons.

These additional features are advantageous in order to increase the range of applications for which the isolated polypeptide can be used.

Preferred solubility factors are maltose binding protein (MPB), SUMO (small-ubiquitin like modifier), StreptagII, a His-tag, SBP (streptavidin binding peptide) and GST (glutathione-S-transferase.

Especially preferred is MBP as an N-terminal tag.

This has the advantage that protein expression and solubility are significantly increased.

Preferred markers are green and red fluorescent proteins (EGFP and mRFP), Gaussia princeps luciferase Gluc.

In various embodiments of this aspect of the present invention the isolated polypeptide comprises a linker fragment inserted between the at least two intein fragments.

In still other various embodiments of this aspect of the present invention the linker fragment is selected from the amino acid sequence MG or the amino acid sequence MGSGGSG.

This has the advantage that protein expression of the contiguous cis splicing polypeptide is improved.

In various embodiments of all aspect of the present invention the isolated polypeptide has the highest splicing yield and splicing rate at 8° C. or 37° C.

In a further aspect the present invention relates an isolated nucleic acid molecule comprising a nucleotide sequence encoding for at least one polypeptide described above or a homolog, mutant or complement thereof.

The term “variant” as used herein refers to a nucleic acid molecule which is substantially similar in structure and biological activity to a nucleic acid molecule according to one of the claimed sequences.

The term “mutant” refers to a nucleic acid molecule the sequence of which has one or more nucleotides added, deleted, substituted or otherwise chemically modified in comparison to a nucleic acid molecule according to one of the claimed sequences, provided always that the mutant retains substantially the same properties as the nucleic acid molecule according to one of the claimed sequences.

As used herein, the term “complement” refers to the complementary nucleic acid of the used/known/discussed nucleic acid. This is an important concept since in molecular biology, complementarity is a property of double-stranded nucleic acids such as DNA and RNA as well as DNA:RNA duplexes. Each strand is complementary to the other in that the base pairs between them are non-covalently connected via two or three hydrogen bonds. Since there is in principle—exceptions apply for thymine/uracil and the tRNA wobble conformation—only one complementary base for any of the bases found in nucleic acids, one can reconstruct a complementary strand for any single strand.

However, for double stranded DNA the term “complement” can also refer to the complementary DNA (cDNA). cDNA can be synthesized from a mature mRNA template in a reaction catalyzed by the enzyme reverse transcriptase.

In various embodiments of this aspect of the present invention the isolated nucleic acid molecule has or comprises SEQ ID NO:19-23.

In further embodiments of this aspect of the invention the isolated nucleic acid molecule is comprised in a vector, preferably a plasmid.

The term “vector”, as used herein, refers to a molecular vehicle used to transfer foreign genetic material into another cell. The vector itself is generally a DNA sequence that consists of an insert (sequence of interest) and a larger sequence. The purpose of a vector to transfer genetic information to another cell is typically to isolate, multiply, or express the insert in the target cell.

The term “plasmid”, as used herein, refers to plasmid vectors, i.e. circular DNA sequences that are capable of autonomous replication within a suitable host due to an origin of replication (“ORI”).

Furthermore, a plasmid may comprise a selectable marker to indicate the success of the transformation or other procedures meant to introduce foreign DNA into a cell and a multiple cloning site which includes multiple restriction enzyme consensus sites to enable the insertion of an insert. Plasmid vectors called cloning or donor vectors are used to ease the cloning and to amplify a sequence of interest. Plasmid vectors called expression or acceptor vectors are specifically for the expression of a gene of interest in a defined target cell. Those plasmid vectors generally show an expression cassette, consisting of a promoter, the transgene and a terminator sequence. Expression plasmids can be shuttle plasmids containing elements that enable the propagation and selection in different host cells.

In further embodiments of this aspect of the at least one isolated nucleic acid molecule is comprised in a host cell.

The term “host cell”, as used herein refers to a transgenic cell, which is used as expression host. Said cell, or its progenitor, has thus been transfected with a suitable vector comprising the cDNA of the protein to be expressed.

In a further aspect the present invention relates to a protein expression system comprising an isolated polypeptide as described above or an isolated nucleic acid molecule as described above expressed from a plasmid in a host cell, e.g. E. coli.

In yet another aspect the present invention relates to a method using an isolated polypeptide as described above or an isolated nucleic acid molecule as described above, wherein the method is selected from the group consisting of modification of a protein, protein lipidation, protein immobilization, protein backbone semi-synthesis, regioselective protein side chain modification and artificial control of protein splicing by light, by a small molecule or a temperature change.

As used herein the term “protein lipidation” refers to the covalent modification of peptides, polypeptides and proteins with a variety of lipids, including fatty acids, isoprenoids, and cholesterol. Lipid modifications play important roles in the localization and function of proteins.

The term “semi-synthesis” as used herein refers to partial chemical synthesis, i.e. a type of chemical synthesis that uses compounds isolated from natural sources (e.g. plant material or bacterial or cell cultures) as starting materials. This is opposed to a total synthesis where large molecules are synthesized from a stepwise combination of small and cheap (usually petrochemical) building blocks.

The term “backbone semi-synthesis” as used herein refers to generation of a protein consisting of a polypeptide segment derived from recombinant protein expression and a segment obtain by organic peptide synthesis.

In a preferred embodiment of this aspect of the present invention the isolated polypeptide is modified in such a way it can be specifically induced to cleave, resulting in a separation of the intein and the N-terminal and/or C-terminal extein sequences. Such a modification can, for example, be achieved via point mutations in at least one of the extein sequences or by omitting one of the extein sequences.

Such a system could, e.g. be used for the purification of proteins with the subsequent cleavage of the employed affinity tag.

Protein trans-splicing (PTS) using inteins is especially well suited for these applications since the protein of interest (POI) will be assembled from two parts (fused as extein sequences to the intein fragments) and each of these fusion constructs can be prepared individually before the PTS reaction. Due to the small size the split intein fragments, one of the intein fusion proteins POI-Int^{N} or Int^{C}-POI can be obtained by using solid-phase peptide synthesis. Alternatively, both fusion proteins can be recombinant but treated individually with bioconjugation chemicals to regioselectively introduce a synthetic label.

Moreover, a method of protein modification employing an isolated polypeptide as described above can be carried out it in complex systems like a living cell or a cell extract.

In various embodiments of this aspect of the present invention the modification is a protein-terminal modification.

In various embodiments of this aspect of the present invention the N-terminal modification is protein labelling.

In a further aspect the present invention relates to a method for the ligation of at least one first peptide to at least one second peptide using an isolated polypeptide as described above or an isolated nucleic acid molecule as described above.

In this context it should be noted that the isolated polypeptide as described above or the isolated nucleic acid molecule as described above, respectively, can be used to generate one of the functional groups for the ligation reaction e.g. for native chemical ligation (NCL) or expressed protein ligation (EPL) reactions or can be employed to create the peptide bonds itself via intein mediated protein trans-splicing.

Thus, in various embodiments of this aspect of the present invention the method comprises covalently linking the N-terminus of the first protein to the C-terminus of the second protein, i.e. it is an intein mediated protein trans-splicing reaction.

In this context the isolated polypeptide described above is advantageous, because it allows for ligation at low concentrations and in the presence of other components.

In yet another aspect the present invention relates to the use of a polypeptide or a nucleic acid molecule as described above, wherein the use is selected from the group consisting of modification of a protein, protein lipidation, protein immobilization, protein backbone semisynthesis, regioselective protein side chain modification, and use as molecular switch. In various embodiments, the inteins and the intein-containing polypeptides described herein can be used for modification of proteins that have therapeutic utility, e.g. are used as pharmaceuticals. Such proteins include, without limitation, antibodies and antibody-like molecules.

In various aspects of this embodiment of the present invention the modification of a protein is selected from site-selective introduction of synthetic moieties into proteins and N-terminal modification.

In various aspects of this embodiment of the invention the described polypeptide or nucleic acids is used protein engineering or protein semi-synthesis.

Moreover, inteins have also been recognized as molecular switches that mediate protein splicing as an output signal only when a certain input signal is given. The latter represents the condition under which the intein is active. By fusing additional polypeptides that mediate a protein—protein interaction to the “free” termini of the split intein fragments the systems can be designed to either work as a biosensor or as an experimental tool to control biological activities that rely on the primary structure of the spliced polypeptide or protein.

In another aspect the present invention relates to a kit comprising a polypeptide or a nucleic acid molecule as described above.

In a preferred embodiment the kit comprises at least one polypeptide as described above comprising an Int^{N} fragment as described above fused to a marker.

In various embodiments of this embodiment of the intention the kit further comprises at least one component selected from the group consisting of at least one vector, at least one resin, DTT, at least one plasmid, at least one expression host, at least one loading buffer, at least one antibody.

As used herein the term “expression host” refers to prokaryotic and eukaryotic expression system hosts, including but not limited to bacteria.

Such a kit is advantageous, inter alia, for ligation and labelling of recombinant proteins, as no proteases, which potentially splice the target protein, have to be used.

In one embodiment of this aspect of the present invention the kit comprises a vector encoding the Int^{C} fragment, into which the protein of interest can be easily cloned. Thus, the Int^{C}-protein of interest fusion protein can be easily expressed.

In addition, in one embodiment of this aspect of the invention the kit comprises the Int^{N} fragment as synthetic peptide fused to a chemical modification desired for the protein of interest, e.g. a fluorophore, biotin etc. In a preferred embodiment the kit comprises the Int^{N} fragment as synthetic peptide fused to a variety of chemical modifications.

In an alternative embodiment the Int^{C} and Int^{N} fragments along with their respective components are comprised in separate kits.

In various embodiments of all aspect of the present invention the splice site of the isolated polypeptide is at amino acids 24-25 from the intein's N-terminal position.

This is advantageous as due to the small size of the N-terminal intein fragment the POI-Int^{N} complex can be obtained by using solid-phase peptide synthesis.

In various embodiments of all aspect of the present invention the N-terminal intein fragment of the isolated polypeptide comprises 24-25 amino acids and the C-terminal intein fragment of the isolated polypeptide comprises 104-105 amino acids.

This property has the advantage that small proteins and especially small intein fragments are more suitable for a range of applications such as chemical or biological, i.e. recombinant protein synthesis.

In various embodiments of all aspect of the present invention the isolated polypeptide is or is comprised in a recombinant protein and/or an antibody and/or a protein hormone.

Other embodiments are within the following non-limiting examples.

### EXAMPLES

### Example 1

### Isolation and Characterization of AceL-TerL

Several closely related phage genes with inteins were identified in metagenomic data from the Antarctic permanently stratified saline Ace Lake (F. M. Lauro, M. Z. DeMaere, S. Yau, M. V. Brown, C. Ng, D. Wilkins, M. J. Raftery, J. A. Gibson, C. Andrews-Pfannkoch, M. Lewis, J. M. Hoffman, T. Thomas, R. Cavicchioli, ISME J. 2011, 5, 879). The genes were of a T4 bacteriophage-type DNA packaging large subunit terminase, and were subsequently termed AceL-TerL inteins (from Ace lake terminase large subunit).

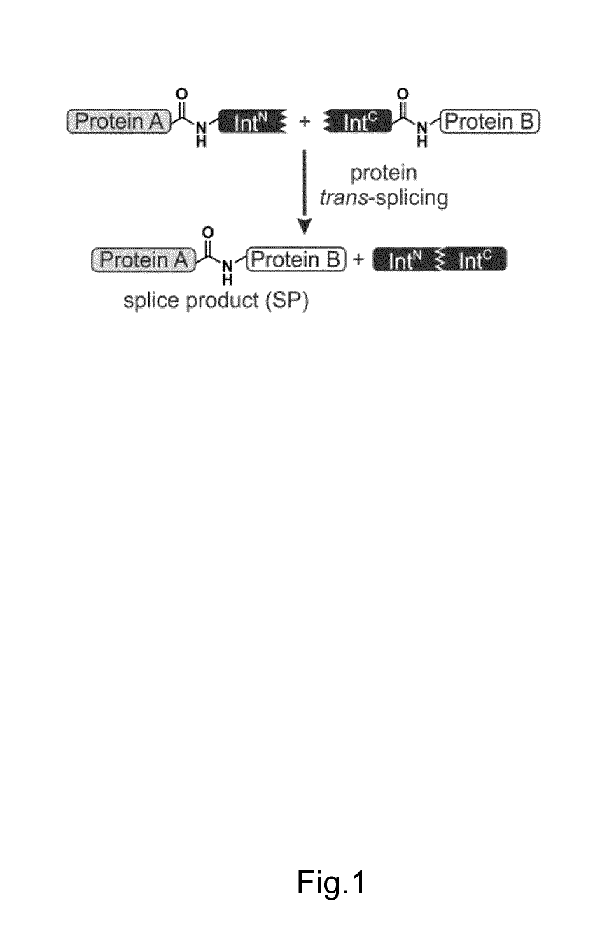

It was shown that these inteins have a novel split site corresponding to a probable surface loop region of the intein with no defined secondary structure following β-strand 3 and α-helix 1 of the typical intein structure (FIG. 2). In contrast, previously reported split sites close to the N-terminal splice junction were all created artificially.

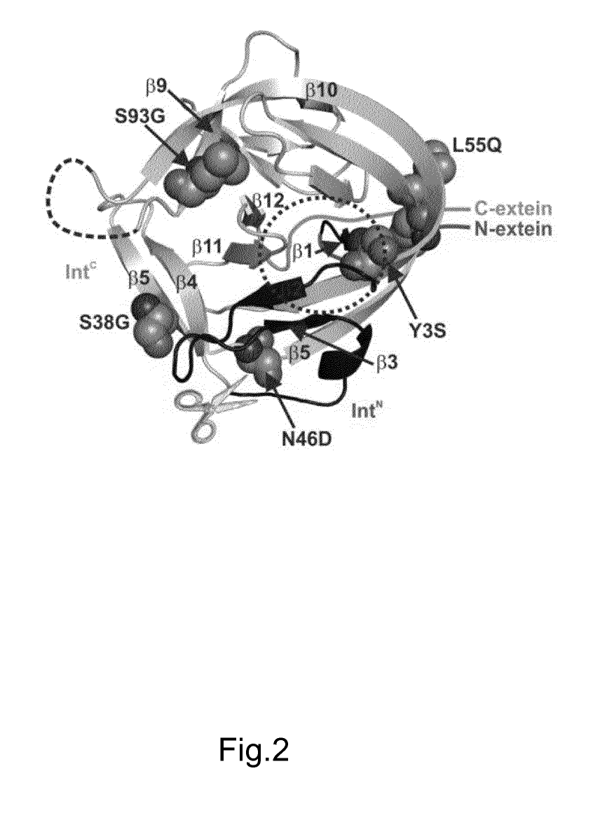

The intein with SEQ ID NO:1, simply termed AceL-TerL intein hereafter, was chosen for functional characterization. To this end, the Int^{N} of 25 aa was prepared by solid-phase peptide synthesis with three native N-extein residues, two lysine residues, and a 5(6)-carboxyfluoresceine moiety (FI-) to give pepWT^{N} (FI-KKEFE-IntN). The Int^{C} (aa 26-129) was recombinantly expressed in E. coli as a fusion with hexahistidine-tagged thioredoxin as a model protein (construct WT^{C}-Trx-H6) and purified using Ni-NTA chromatography. Upon mixing of the two components spontaneous protein trans-splicing was observed (FIG. 3). The formation of the splice product (SP) FI-KKEFE-Trx-H6 and of the excised intein fragments as by-products indicated that this intein was fully active.

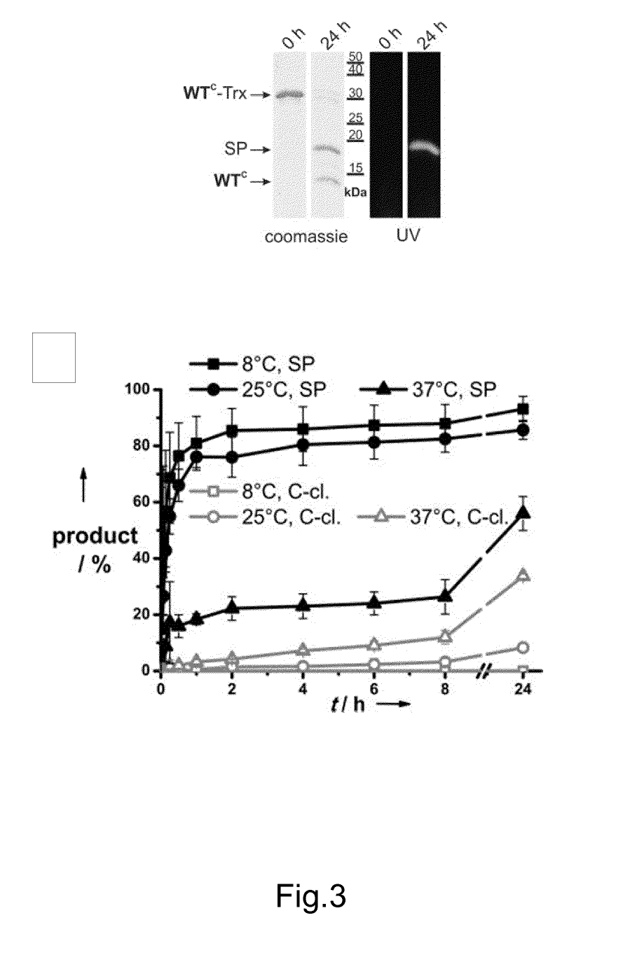

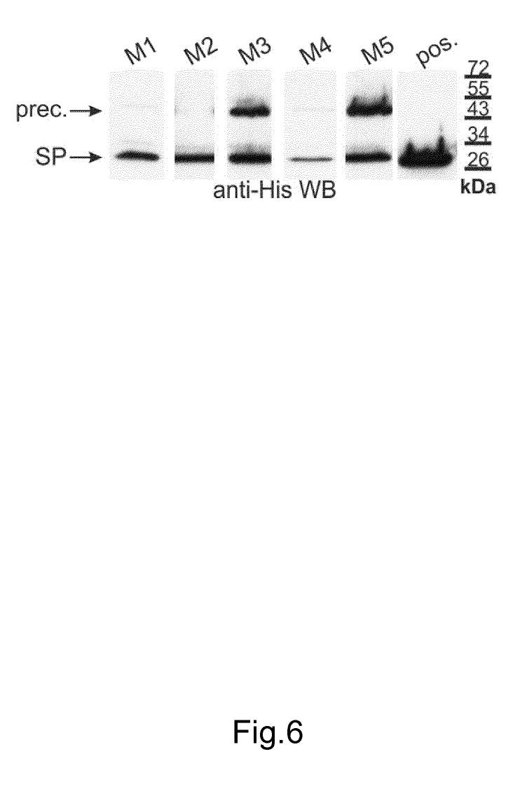

As demonstrated in (FIG. 7) a clear temperature dependence was observed from splicing assays at 8° C., 25° C., and 37° C., with the highest rate at 8° C. (k=1.7±0.2×10⁻³ s⁻¹; t½=7.2±1.1 min) and a ˜50-fold slower rate at 37° C. Importantly, no C-terminal cleavage side-product (Trx-H6) could be detected at 8° C. (FIG. 3 and FIG. 4). Such cleavage products are often observed for engineered inteins and may significantly limit the practical utility of the intein system. Together, these results indicated the excellent potential of this naturally split intein for protein trans-splicing applications, also aided by its total length of only 129 aa, being one of the shortest known inteins. The rate and yield of about 90% of the AceL-TerL intein at 8° C. is remarkable.

### Example 2

### Modification of Wild-Type AceL-TerL

Although low temperatures like 8° C. appear ideal to preserve potentially fragile proteins of interest in in vitro experiments, expression of the intein fusion proteins in E. coli has to be performed at higher temperatures. Furthermore, inteins with higher thermostability should be beneficial for high activity in diverse sequence contexts, potentially also at lower temperatures, and for cellular applications.

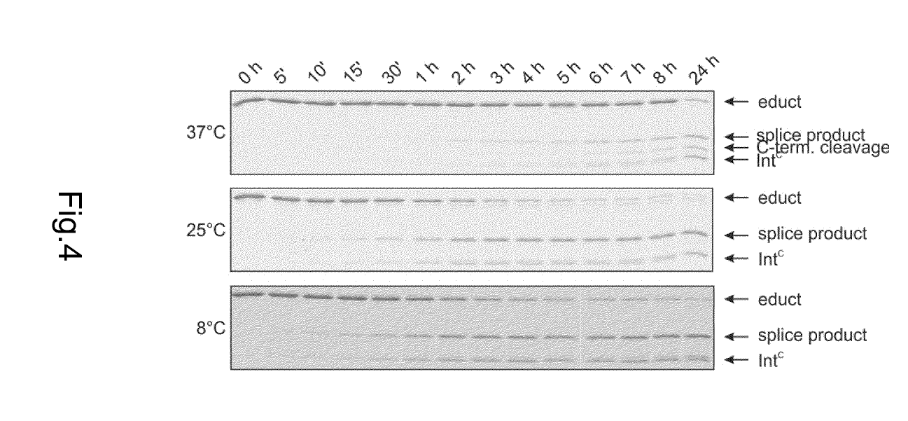

Thus, in order to generate modified AceL-TerL inteins with a higher activity at 37° C., the AceL-TerL intein was converted into a contiguous, cis-splicing intein by fragment fusion (FIG. 5) and inserted on the DNA level into the KanR gene. Active intein alleles capable of splicing out of the translated gene product can be selected because they render the host E. coli cells resistant to the antibiotic kanamycin. The non-mutated intein gave rise to colony growth at 25° C. under selective conditions, but not at 37° C. This finding correlated with protein splicing activity determined by Western blot analyses (data not shown). These results provided the basis for selection by temperature.

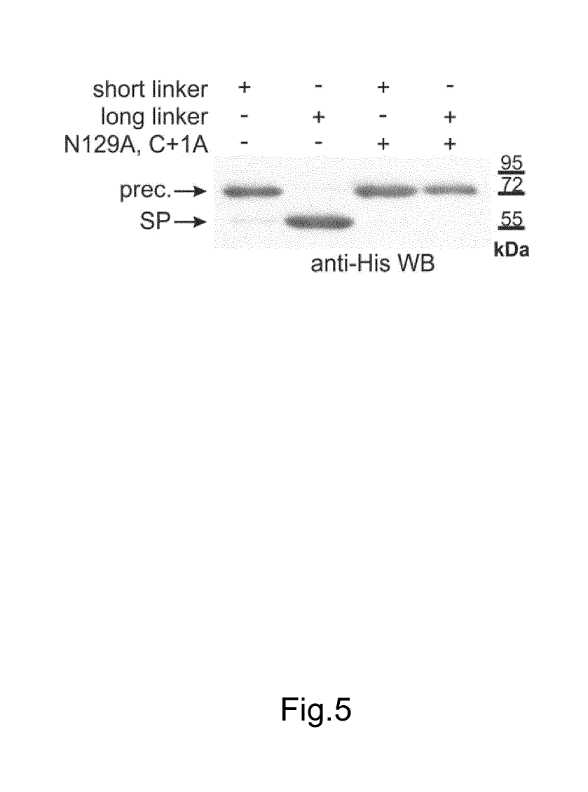

Subsequently, a library encoding mutant inteins was created using error prone PCR (epPCR) and used to transform E. coli cells. Randomly picked kanamycin-resistant colonies that were selected on plates at 37° C. were then re-streaked on plates with kanamycin concentrations of up to 150 μg/ml. Plasmids isolated from resistant clones were analyzed by DNA-sequencing. Five different mutant inteins, termed M1 to M5 (Table 1), were selected and confirmed by Western blotting to have acquired splicing activity at this elevated temperature (FIG. 6). The mutant inteins contained one to four amino acid substitutions, in both the IntN and IntC parts (Table 1). To discern the effect of individual mutations, an additional construct with the single L55Q mutation, termed M6 (Table 1), was created by site-directed mutagenesis.

TABLE 1
Mutations in the modified AceL-TertL intein fragments

| Mutant Name | Int^{N} | Int^{C} |
|---|---|---|
| M1 | A25T | N46D, L55Q |
| M2 | — | S38G, N46I, N54D, L55Q |
| M3 | Y3S | S93G |
| M4 | — | N46I, L55Q |
| M5 | — | N46D |
| M6 | — | L55Q |
| MX1 | — | N46D, L55Q |
| M31 | Y3S | N46D, L55Q |

### Example 3

### Characterization of the Modified AceL-TerL Intein Fragments

The effect of the mutations M1 to M6 was investigated. The Int^{C} fragments of the M1 to M6 mutants, termed M1^{C} to M6^{C}, were expressed and purified as Int^{C}-Trx-H6 fusion proteins, and the Int^{N} parts of the M1 and M3 mutants, termed M1^{N} and M3^{N}, were included in two synthetic peptides of the format FI-KKEFE-IntN (pepM1^{N} and pepM3^{N}, respectively). A clear improvement over the wild-type intein protein trans-splicing reactions at 37° C. could be observed for all the mutants. Rates were increased by about 2- to 14-fold (FIG. 9 and FIG. 11) and yields were increased to 65-85% after 24 h compared to about 60% for the wild-type intein, with concomitant decrease of the C-cleavage product (FIG. 9).

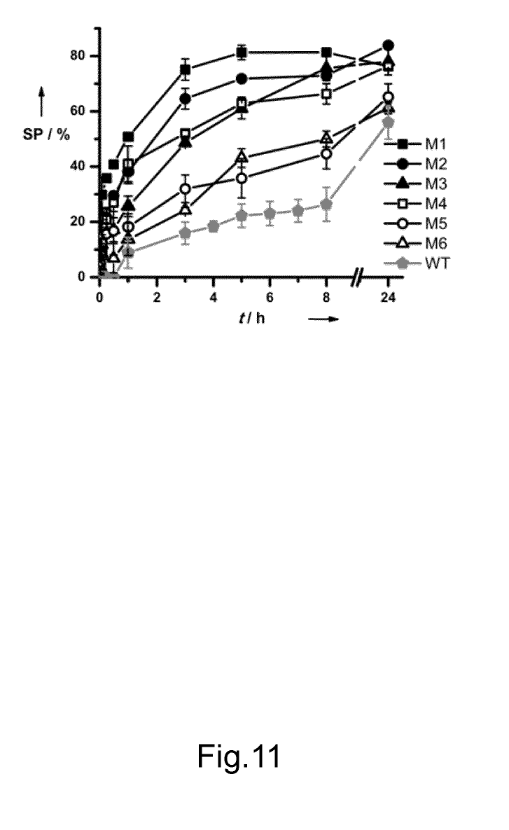

Moreover, the mutations seemed to have additive effects, as exemplified by the observation that the combined N46D and L55Q mutations (pepWT^{N}+M1^{C}) resulted in higher rates than the individual mutations (pepWT^{N}+M5^{C} and pepWT^{N}+M6^{C}, respectively) (FIG. 10 and FIG. 12).

### Example 4

### Further Optimization of the Modified AceL-TerL Intein Fragments

The Int^{N}-mutation of the M3 mutant (Y3S) were combined with the Int^{C}-mutations of the M1 (N46D, L55Q) or M2 (S38G, N461, N54D, L55Q) mutants (pepM3N+M1C and pepM3N+M2C). These combinations spliced ˜29-fold and ˜56-fold faster, respectively, than the wild-type at the selection temperature of 37° C. (FIG. 10).

As the combinations of pepM3^{N}+M1^{C} and pepWT^{N}+M1^{C} surprisingly demonstrated a superior ratio between splicing and cleavage yields these were chosen for subsequent experiments. They were termed MX1 mutant (pepWT^{N}+M1^{C}) and M31 mutant (pepM3^{N}+M1^{C}) (Table 1 and FIG. 10).

### Example 5

### N-Terminal Chemical Modification of Proteins

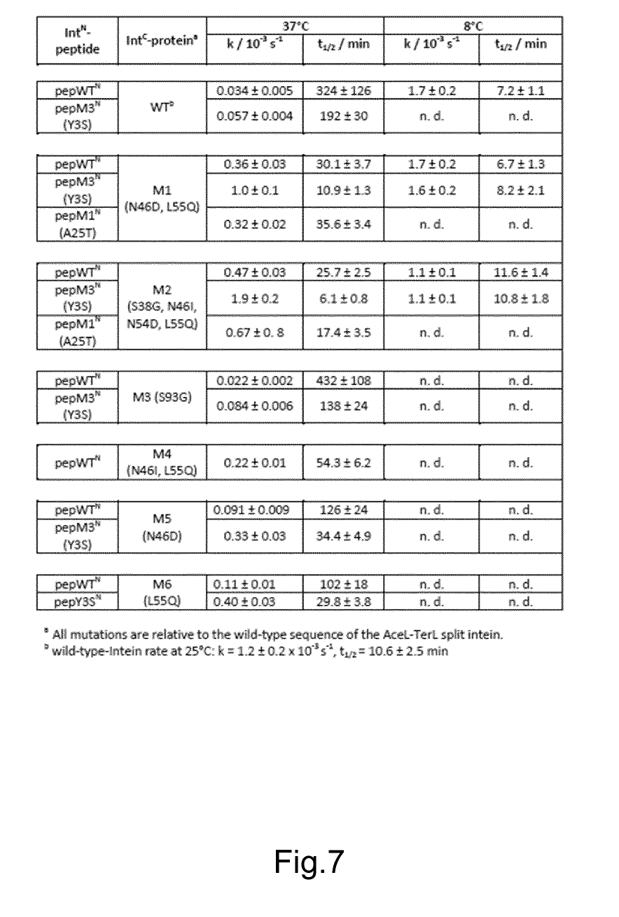

The maltose binding protein (MBP) was included as an N-terminal tag to give the construct MBP-Int^{C}-TycB1 with the AceL-TerL MX1 and M31. MBP improved semi-synthetic protein trans-splicing of the wild-type AceL-TerL intein and mutants (data not shown). In particular, the MX1 mutant was efficiently expressed, well soluble, and spliced at 8° C. to give yields of 80-95% with a 13-fold higher rate than the M86 DnaB mutant intein and a 15-fold higher rate than the unevolved wild-type AceL-TerL intein (FIG. 8). Similar results were obtained in a detailed kinetic study using Trx as the protein of interest (FIG. 8).

### Example 6

### Chemical Modification Using AceL-TerL Mutants

In order to demonstrate the applicability of the AceL-TerL intein mutants for the chemical modification of a diverse range of proteins of interest, the MX1 mutant was fused with green and red fluorescent proteins EGFP and mRFP, Gaussia princeps luciferase Gluc, as well as the murine E2 conjugating enzyme Ubc9 and the human protease SENP1 from the SUMO pathway.

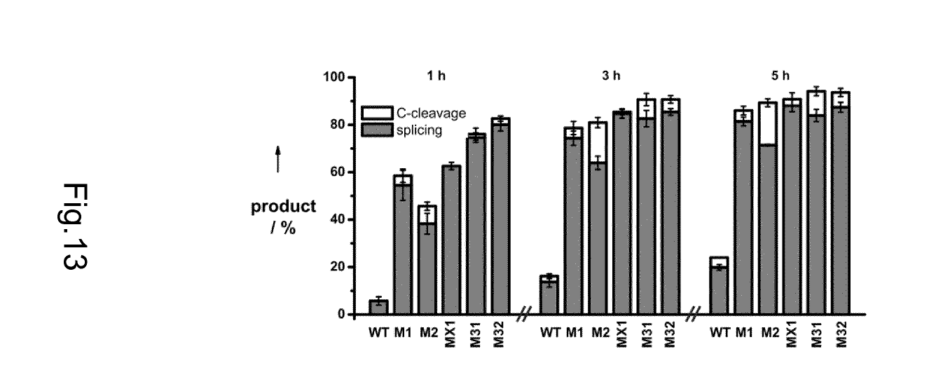

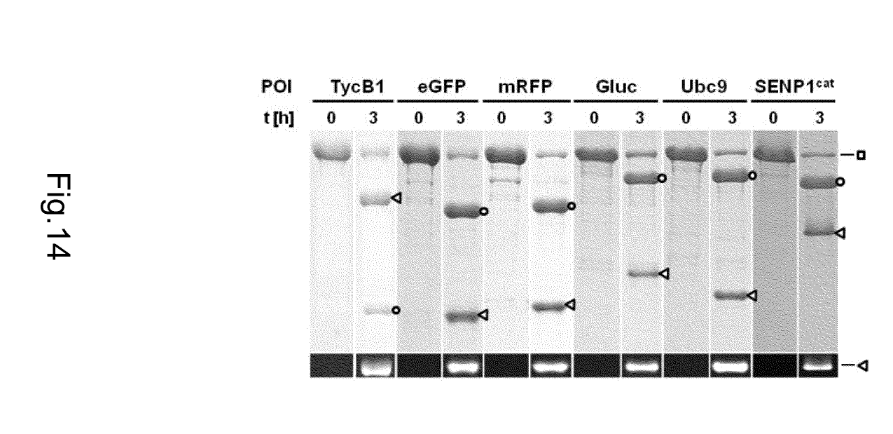

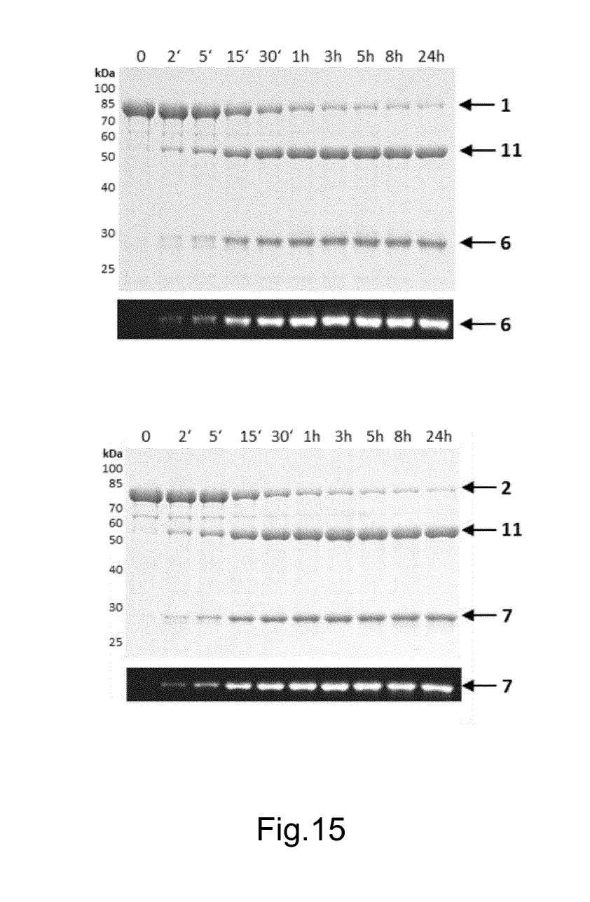

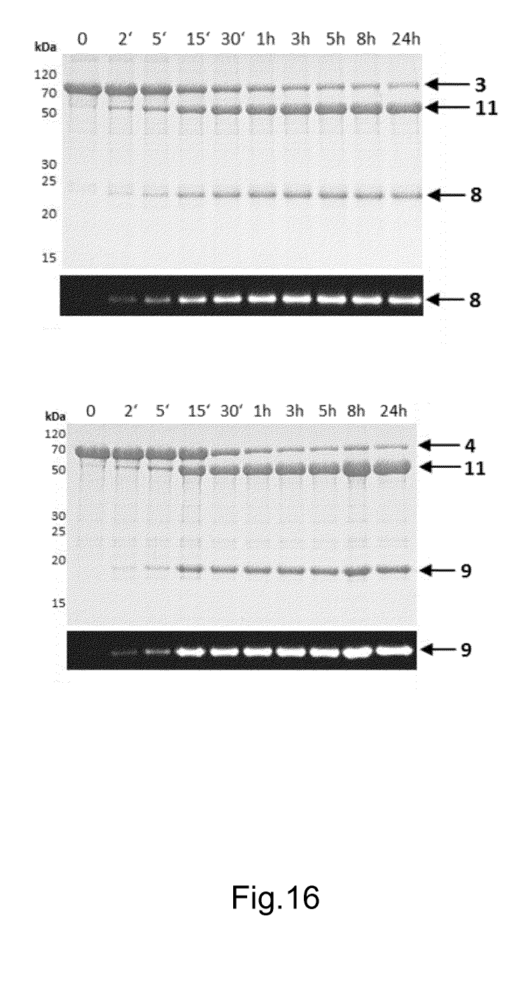

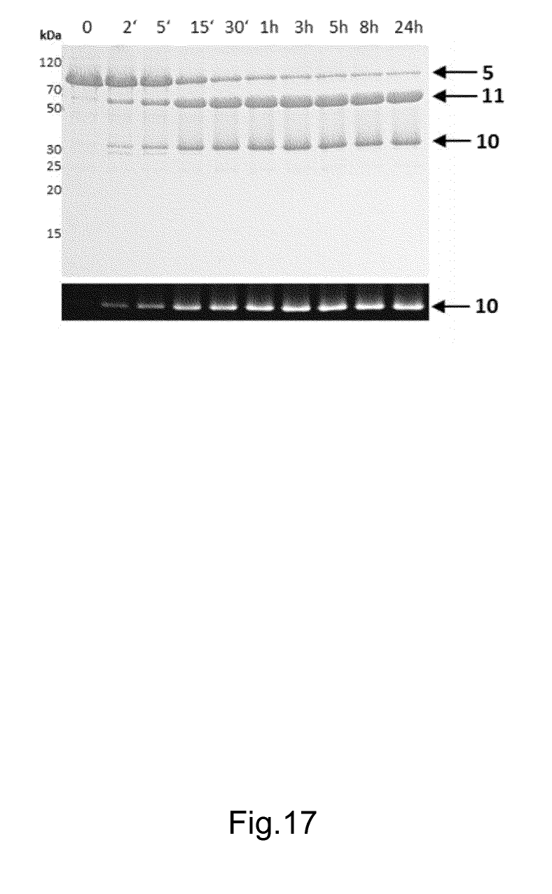

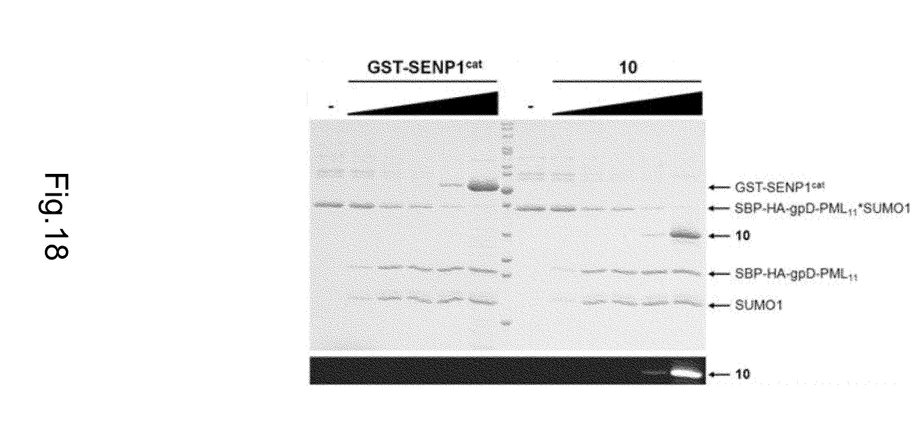

As shown in FIGS. 14-18, all tested proteins were efficiently modified with the synthetic fluorophore at the N-terminus, with ˜80% yields of the desired conjugates after 3 h at 8° C. For a biochemical characterization of the proteins, the fluorescently labeled enzymes TycB1 and SENP1 were prepared and purified on a preparative scale and could be shown to be fully catalytically active (FIGS. 15-18). These labeled proteins will prove useful in future biophysical studies.

### Example 7

### Characterization of the VidaL T4Lh-1 intein

The Int^{C} encoding fragment of the VidaL_T4Lh-1 intein (nt: SEQ ID NO:121; aa: SEQ ID NO:117) was cloned into an expression vector coding for the construct SBP-(VidaL_T4Lh-1)^{C}-Trx-His₆ (SBP=streptavidin binding peptide, Trx=thioredoxin, His₆=hexahistidine tag; amino acid (aa) sequence: SEQ ID NO:197; nucleotide (nt) sequence: SEQ ID NO:198). The protein was produced by overexpression in E. coli and purified from the supernatant after cell lysis using streptactin affinity chromatography. The Int^{N} fragment of the intein (aa: SEQ ID NO:116) was synthesized by solid-phase peptide synthesis as a part of the peptide

FI-LASCVHPDTKVTIRRKLC-OH (SEQ ID NO: 199; FI =
5,6-Carboxyfluoresceine; Int^{N} sequence
underlined).

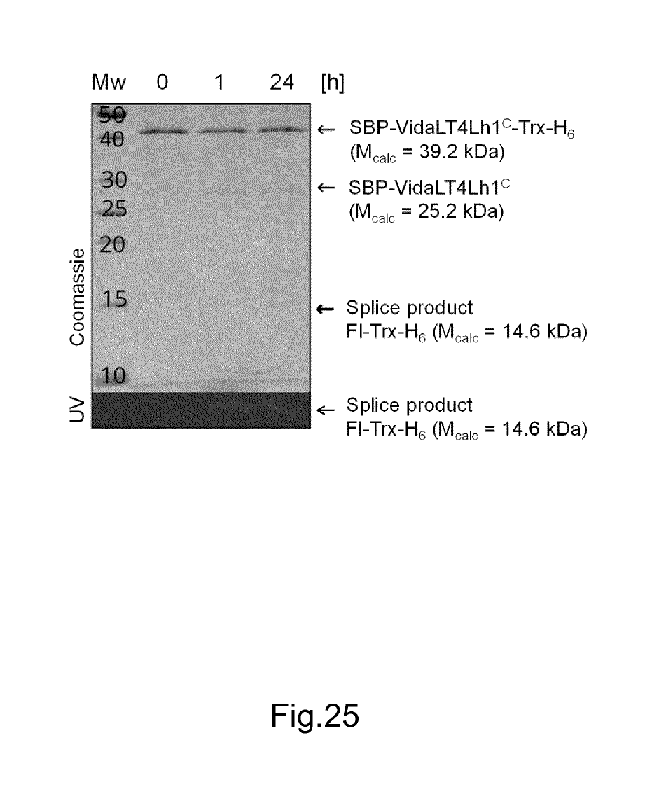

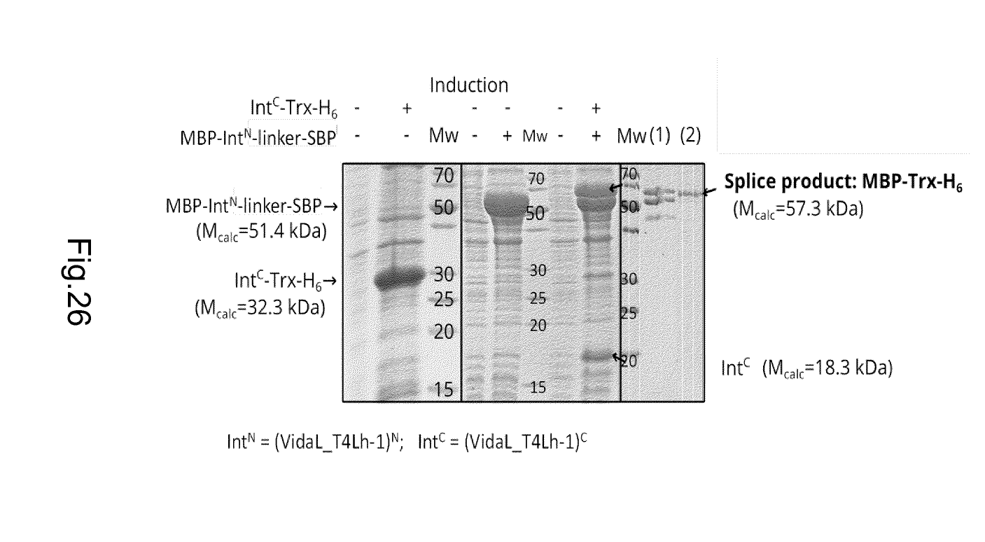

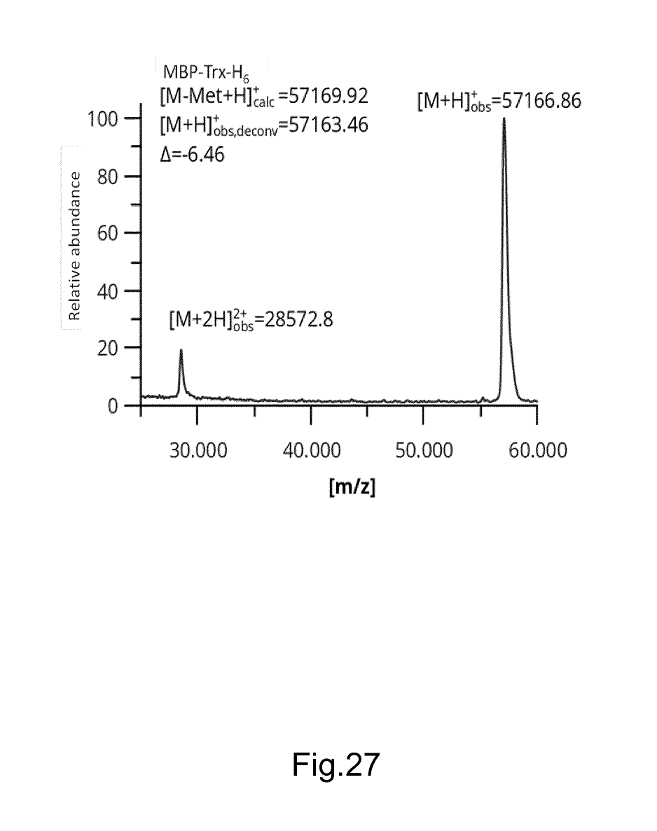

Following mixing of both fragments (concentrations for Int^{N}-construct 9 μM, for Int^{C}-fragment 9 μM) in splice buffer (50 mM Tris/HCl, 300 mM NaCl, 1 mM EDTA, pH 7.0) with 2 mM TCEP (tris-carboxyethylphosphine) incubation was carried out at 25° C. Aliquots were removed at the indicated time points and quenched by mixing with SDS PAGE sample buffer and boiling at 95° C. for 5 min. Shown is an analysis of the samples on a Coomassie-stained SDS PAGE gel (see FIG. 25; Mw=molecular weight marker). Before staining, the gel was also photographed under UV illumination, which revealed the fluorescently labeled band of the splice product (lower panel in FIG. 25). Formation of the expected new protein bands demonstrated the activity of the intein in semisynthetic protein trans-splicing. The split intein was also reconstituted by co-expression two constructs, containing either the Int^{N} or the Int^{C} fragment, in E. coli cells and observing protein trans-splicing in the cell extract of the cells. FIG. 26 shows a Coomassie-stained SDS PAGE gel, in which the expression of the individual (VidaL_T4Lh-1)^{C}-Trx-His₆ construct (aa: SEQ ID NO:200; nt: SEQ ID NO:201), the expression of the individual MBP-(VidaL_T4Lh-1)^{N}-linker-SBP construct (SBP=streptavidin binding peptide) (aa: SEQ ID NO:202; nt: SEQ ID NO:203), and the co-expression of both constructs is shown (from left to right; Mw=molecular weight marker). The new band appearing at 57.3 kDa is the splice product MBP-Trx-His₆. The two lanes labeled with (1) and (2) show the purified splice product after an amylose column (1) and a Ni-NTA column (2). The protein sample shown in lane (2) was then used for analysis by mass spectrometry, which further confirmed the identity of the splice product MBP-Trx-His₆ (FIG. 27; all masses shown all average masses).

### Example 8

### Characterization of the VidaL UvsX-2 Intein

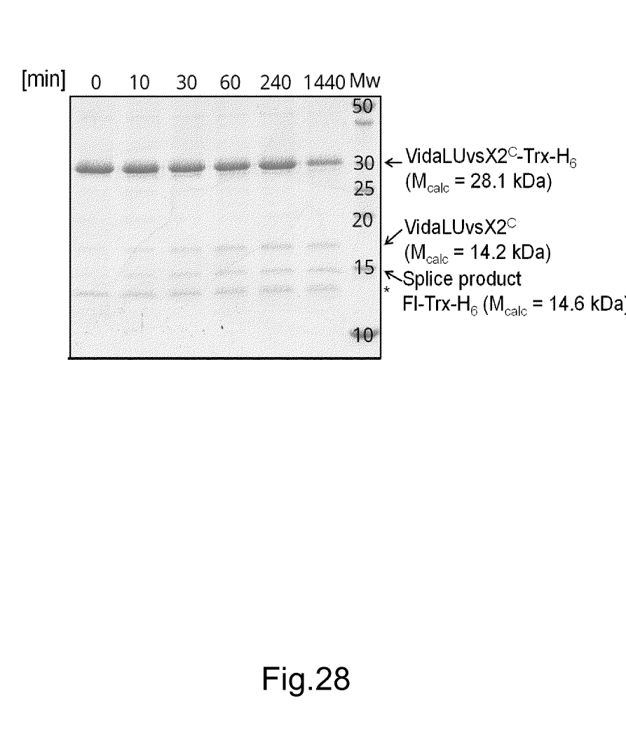

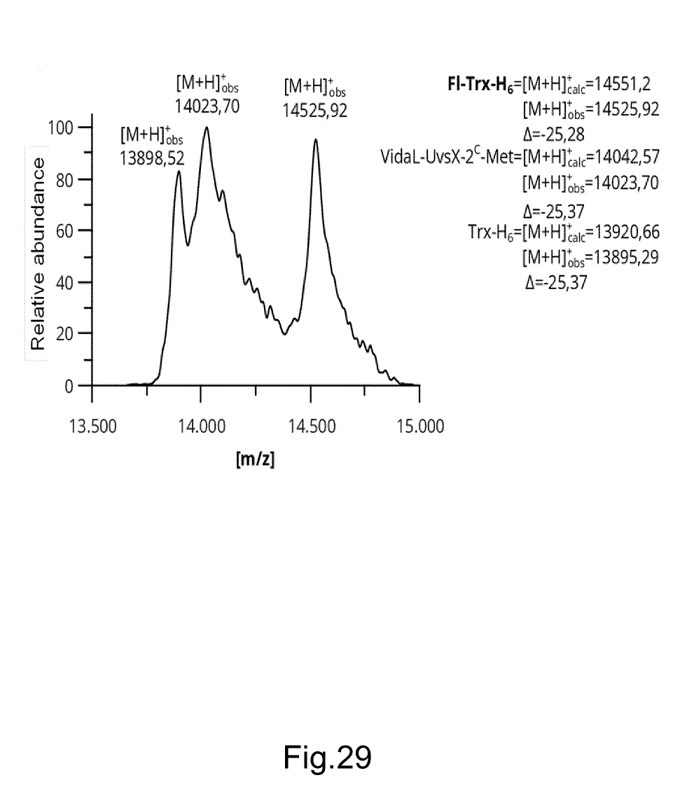

The Int^{C} encoding fragment of the VidaL_UvsX-2 intein (nt: SEQ ID NO:127; aa: SEQ ID NO:123) was cloned into an expression vector coding for the construct (VidaL_UvsX-2)^{C}-Trx-His₆ (Trx=thioredoxin, His₆=hexahistidine tag; aa: SEQ ID NO:204; nt: SEQ ID NO:205. The protein was produced by overexpression in E. coli and purified using Ni-NTA-chromatography. The Int^{N} fragment of the intein (SEQ ID NO:122) was synthesized by solid-phase peptide synthesis as a part of the peptide FI-ESGCLPKEAVVQIRLTKKGA-OH (SEQ ID NO:206; FI=5,6-Carboxyfluoresceine; Int^{N} sequence underlined). Following mixing of both fragments (concentrations for Int^{N}-construct 15 μM, for Int^{C}-fragment 15 μM) in splice buffer (50 mM Tris/HCl, 300 mM NaCl, 1 mM EDTA, pH 7.0) with 2 mM TCEP (tris-carboxyethylphosphine) incubation was carried out at 25° C. Aliquots were removed at the indicated time points and quenched by mixing with SDS PAGE sample buffer and boiling at 95° C. for 5 min. Shown is an analysis of the samples on a Coomassie-stained SDS PAGE gel (see FIG. 28; *=protein contamination; Mw=molecular weight marker). Formation of the expected new protein bands demonstrated the activity of the intein in semisynthetic protein trans-splicing. Furthermore, the molecular mass of the splice product FI-Trx-H6 as confirmed by a mass spectrometric analysis of the reaction mixture (see FIG. 29; average masses are given).

In summary, novel inteins with an unusually short N-terminal fragment of only 15, 16 or 25 amino acids were identified and significantly improved mutants of these intein were generated. These intein fragments and the corresponding inteins respectively, can serve as powerful and generally applicable tools for the N-terminal chemical modification of proteins using semisynthetic protein trans-splicing. Advantages of this approach over chemical ligation reactions include the low required reactant concentrations, the absence of non-proteinogenic functional groups to facilitate the reaction, and the orthogonality to the cellular chemical environment. The high activity of the new split inteins at low temperatures like 8° C. is of particular advantage for in vitro labelling experiments with fragile proteins.

TABLE 2
Sequences overview:

| SEQ ID | | |
|---|---|---|
| NO: | Name | Sequence |
| 1 | aa | CVYGDTMVETEDGKIKIEDLYKRLAMFRTNTNNIKILSPNGFSNFN |
| | WTAceL-TerL-11 | GIQKVERNLYQHIIFDDDTEIKTSINHPFGKDKILARDVKVGDYLNS |
| | | KKVLYNELVNENIFLYDPINVEKESLYITNGVVSHN |
| 2 | aa WTAceL-TerL-3 IntN | CVDGNTIVETEDGKIKIEDLYKKL |
| 3 | aa WTAceL-TerL-4 Int^{N} | CVDGNTIVETEDGKIKIEDLYKKM |
| 4 | aa WTAceL-TerL-5 Int^{N} | CVDGNTIVETEDGKIKIEDLYKKL |
| 5 | Aa WTAceL-TerL-11 Int^{N} | CVYGDTMVETEDGKIKIEDLYKRLA |
| 6 | aa | MFRTNTNNIKILSPNGFSNFNGIQKVERNLYQHIIFDDDTEIKTSINH |
| | WTAceL-TerL-11 Int^{C} | PFGKDKILARDVKVGDYLNSKKVLYNELVNENIFLYDPINVEKESL |
| | | YITNGVVSHN |
| 7 | aa | MEIEEGKLVIWINGDKGYNGLAEVGKKFEKDTGIKVTVEHPDKLEE |
| | (MBP-AceL-TerL^{cis}-FKBP- | KFPQVAATGDGPDIIFWAHDRFGGYAQSGLLAEITPDKAFQDKLYP |
| | His6) | FTWDAVRYNGKLIAYPIAVEALSLIYNKDLLPNPPKTWEEIPALDKE |
| | pIT063 | LKAKGKSALMFNLQEPYFTWPLIAADGGYAFKYENGKYDIKDVG |
| | | VDNAGAKAGLTFLVDLIKNKHMNADTDYSIAEAAFNKGETAMTIN |
| | | GPWAWSNIDTSKVNYGVTVLPTFKGQPSKPFVGVLSAGINAASPN |
| | | KELAKEFLENYLLTDEGLEAVNKDKPLGAVALKSYEEELAKDPRIA |
| | | ATMENAQKGEIMPNIPQMSAFWYAVRTAVINAASGRQTVDEALK |
| | | DAQTNSSSNNNNNNNNNNLGIEGRISEFEFECVYGDTMVETEDGKI |
| | | KIEDLYKRLAMGMFRTNTNNIKILSPNGFSNFNGIQKVERNLYQHII |
| | | FDDDTEIKTSINHPFGKDKILARDVKVGDYLNSKKVLYNELVNENI |
| | | FLYDPINVEKESLYITNGVVSHNCEFLSRNNGNGNGTRGVQVETISP |
| | | GDGRTFPKRGQTCVVHYTGMLEDGKKFDSSRDRNKPFKFMLGKQ |
| | | EVIRGWEEGVAQMSVGQRAKLTISPDYAYGATGHPGIIPPHATLVF |
| | | DVELLKLETSYGSRSHHHHHH |
| 8 | aa | MEIEEGKLVIWINGDKGYNGLAEVGKKFEKDTGIKVTVEHPDKLEE |
| | (MBP-AceL-TerL^{cis}-FKBP- | KFPQVAATGDGPDIIFWAHDRFGGYAQSGLLAEITPDKAFQDKLYP |
| | His6) | FTWDAVRYNGKLIAYPIAVEALSLIYNKDLLPNPPKTWEEIPALDKE |
| | pIT064 | LKAKGKSALMFNLQEPYFTWPLIAADGGYAFKYENGKYDIKDVG |
| | | VDNAGAKAGLTFLVDLIKNKHMNADTDYSIAEAAFNKGETAMTIN |
| | | GPWAWSNIDTSKVNYGVTVLPTFKGQPSKPFVGVLSAGINAASPN |
| | | KELAKEFLENYLLTDEGLEAVNKDKPLGAVALKSYEEELAKDPRIA |
| | | ATMENAQKGEIMPNIPQMSAFWYAVRTAVINAASGRQTVDEALK |
| | | DAQTNSSSNNNNNNNNNNLGIEGRISEFEFECVYGDTMVETEDGKI |
| | | KIEDLYKRLAMGSGGSGMFRTNTNNIKILSPNGFSNFNGIQKVERN |
| | | LYQHIIFDDDTEIKTSINHPFGKDKILARDVKVGDYLNSKKVLYNEL |
| | | VNENIFLYDPINVEKESLYITNGVVSHNCEFLSRNNGNGNGTRGVQ |
| | | VETISPGDGRTFPKRGQTCVVHYTGMLEDGKKFDSSRDRNKPFKF |
| | | MLGKQEVIRGWEEGVAQMSVGQRAKLTISPDYAYGATGHPGIIPP |
| | | HATLVFDVELLKLETSYGSRSHHHHHH |
| 9 | pIT065: inaktiv(N129A,C + 1A) | MEIEEGKLVIWINGDKGYNGLAEVGKKFEKDTGIKVTVEHPDKLEE |
| | | KFPQVAATGDGPDIIFWAHDRFGGYAQSGLLAEITPDKAFQDKLYP |
| | | FTWDAVRYNGKLIAYPIAVEALSLIYNKDLLPNPPKTWEEIPALDKE |
| | | LKAKGKSALMFNLQEPYFTWPLIAADGGYAFKYENGKYDIKDVG |
| | | VDNAGAKAGLTFLVDLIKNKHMNADTDYSIAEAAFNKGETAMTIN |
| | | GPWAWSNIDTSKVNYGVTVLPTFKGQPSKPFVGVLSAGINAASPN |
| | | KELAKEFLENYLLTDEGLEAVNKDKPLGAVALKSYEEELAKDPRIA |
| | | ATMENAQKGEIMPNIPQMSAFWYAVRTAVINAASGRQTVDEALK |
| | | DAQTNSSSNNNNNNNNNNLGIEGRISEFEFECVYGDTMVETEDGKI |
| | | KIEDLYKRLAMGMFRTNTNNIKILSPNGFSNFNGIQKVERNLYQHII |
| | | FDDDTEIKTSINHPFGKDKILARDVKVGDYLNSKKVLYNELVNENI |
| | | FLYDPINVEKESLYITNGVVSHAAEFLSRNNGNGNGTRGVQVETISP |
| | | GDGRTFPKRGQTCVVHYTGMLEDGKKFDSSRDRNKPFKFMLGKQ |
| | | EVIRGWEEGVAQMSVGQRAKLTISPDYAYGATGHPGIIPPHATLVF |
| | | DVELLKLETSYGSRSHHHHHH |
| 10 | pIT066: inaktiv(N129A,C + 1A) | MEIEEGKLVIWINGDKGYNGLAEVGKKFEKDTGIKVTVEHPDKLEE |
| | | KFPQVAATGDGPDIIFWAHDRFGGYAQSGLLAEITPDKAFQDKLYP |
| | | FTWDAVRYNGKLIAYPIAVEALSLIYNKDLLPNPPKTWEEIPALDKE |
| | | LKAKGKSALMFNLQEPYFTWPLIAADGGYAFKYENGKYDIKDVG |
| | | VDNAGAKAGLTFLVDLIKNKHMNADTDYSIAEAAFNKGETAMTIN |
| | | GPWAWSNIDTSKVNYGVTVLPTFKGQPSKPFVGVLSAGINAASPN |
| | | KELAKEFLENYLLTDEGLEAVNKDKPLGAVALKSYEEELAKDPRIA |
| | | ATMENAQKGEIMPNIPQMSAFWYAVRTAVINAASGRQTVDEALK |
| | | DAQTNSSSNNNNNNNNNNLGIEGRISEFEFECVYGDTMVETEDGKI |
| | | KIEDLYKRLAMGSGGSGMFRTNTNNIKILSPNGFSNFNGIQKVERN |
| | | LYQHIIFDDDTEIKTSINHPFGKDKILARDVKVGDYLNSKKVLYNEL |
| | | VNENIFLYDPINVEKESLYITNGVVSHAAEFLSRNNGNGNGTRGVQ |
| | | VETISPGDGRTFPKRGQTCVVHYTGMLEDGKKFDSSRDRNKPFKF |
| | | MLGKQEVIRGWEEGVAQMSVGQRAKLTISPDYAYGATGHPGIIPP |
| | | HATLVFDVELLKLETSYGSRSHHHHHH |
| 11 | aa M1^{N} | CVYGDTMVETEDGKIKIEDLYKRLT |
| 12 | aa M3^{N} | CVSGDTMVETEDGKIKIEDLYKRLA |
| 13 | aa M1^{C} | MFRTNTNNIKILSPNGFSNFDGIQKVERNQYQHIIFDDDTEIKTSINH |
| | | PFGKDKILARDVKVGDYLNSKKVLYNELVNENIFLYDPINVEKESL |
| | | YITNGVVSHN |
| 14 | aa M2^{C} | MFRTNTNNIKILGPNGFSNFIGIQKVERDQYQHIIFDDDTEIKTSINHP |
| | | FGKDKILARDVKVGDYLNSKKVLYNELVNENIFLYDPINVEKESLY |
| | | ITNGVVSHN |
| 15 | aa M3^{C} | MFRTNTNNIKILSPNGFSNFNGIQKVERNLYQHIIFDDDTEIKTSINH |
| | | PFGKDKILARDVKVGDYLNGKKVLYNELVNENIFLYDPINVEKESL |
| | | YITNGVVSHN |
| 16 | aa M4^{C} | MFRTNTNNIKILSPNGFSNFIGIQKVERNQYQHIIFDDDTEIKTSINHP |
| | | FGKDKILARDVKVGDYLNSKKVLYNELVNENIFLYDPINVEKESLY |
| | | ITNGVVSHN |
| 17 | aa M5^{C} | MFRTNTNNIKILSPNGFSNFDGIQKVERNLYQHIIFDDDTEIKTSINH |
| | | PFGKDKILARDVKVGDYLNSKKVLYNELVNENIFLYDPINVEKESL |
| | | YITNGVVSHN |
| 18 | aa M6^{C} | MFRTNTNNIKILSPNGFSNFNGIQKVERNQYQHIIFDDDTEIKTSINH |
| | | PFGKDKILARDVKVGDYLNSKKVLYNELVNENIFLYDPINVEKESL |
| | | YITNGVVSHN |
| 19 | DNA M1^{C} | ATGTTTCGTACCAACACCAACAACATTAAAATTCTGAGCCCGAA |
| | | CGGCTTTAGTAACTTTGACGGCATTCAGAAAGTGGAACGTAACC |
| | | AGTATCAGCATATTATTTTTGATGATGATACCGAAATTAAAACC |
| | | AGCATTAACCATCCGTTTGGCAAAGATAAAATTCTGGCGCGTGA |
| | | TGTGAAAGTGGGCGATTATCTGAACAGCAAAAAAGTGCTGTATA |
| | | ACGAACTGGTGAACGAAAACATTTTTCTGTATGATCCGATTAAC |
| | | GTGGAAAAAGAAAGCCTGTATATTACCAACGGCGTGGTGAGCC |
| | | ATAAC |
| 20 | DNA M2^{C} | ATGTTTCGTACCAACACCAACAACATTAAAATTCTGGGCCCGAA |
| | | CGGCTTTAGCAACTTTATCGGCATTCAGAAAGTGGAACGTGACC |
| | | AGTATCAGCATATTATTTTTGATGATGATACCGAAATTAAAACC |
| | | AGCATTAACCATCCGTTTGGCAAAGATAAAATTCTGGCGCGTGA |
| | | TGTGAAAGTGGGCGATTATCTGAACAGCAAAAAAGTGCTGTATA |
| | | ACGAACTGGTGAACGAAAACATTTTTCTGTATGATCCGATTAAC |
| | | GTGGAAAAAGAAAGCCTGTATATTACCAACGGCGTGGTGAGCC |
| | | ATAAC |
| 21 | DNA M3^{C} | ATGTTTCGTACCAACACCAACAACATTAAAATTCTGAGCCCGAA |
| | | CGGCTTTAGCAACTTTAACGGCATTCAGAAAGTGGAACGTAACC |
| | | TGTATCAGCATATTATTTTTGATGATGATACCGAAATTAAAACC |
| | | AGCATTAACCATCCGTTTGGCAAAGATAAAATTCTGGCGCGTGA |
| | | TGTGAAAGTGGGCGATTATCTGAACGGCAAAAAAGTGCTGTATA |
| | | ACGAACTGGTGAACGAAAACATTTTTCTGTATGATCCGATTAAC |
| | | GTGGAAAAAGAAAGCCTGTATATTACCAACGGCGTGGTGAGCC |
| | | ATAAC |
| 22 | DNA M4^{C} | ATGTTTCGTACCAACACCAACAACATTAAAATTCTGAGCCCGAA |
| | | CGGCTTTAGCAACTTTATCGGCATTCAGAAAGTGGAACGTAACC |
| | | AGTATCAGCATATTATTTTTGATGATGATACCGAAATTAAAACC |
| | | AGCATTAACCATCCGTTTGGCAAAGATAAAATTCTGGCGCGTGA |
| | | TGTGAAAGTGGGCGATTATCTGAACAGCAAAAAAGTGCTGTATA |
| | | ACGAACTGGTGAACGAAAACATTTTTCTGTATGATCCGATTAAC |
| | | GTGGAAAAGGAAAGCCTGTATATTACCAACGGCGTGGTGAGCC |
| | | ATAAC |
| 23 | DNA M5^{C} | ATGTTTCGTACCAACACCAACAACATTAAAATTCTGAGCCCGAA |
| | | CGGCTTTAGCAACTTTGACGGCATTCAGAAAGTGGAACGTAACC |
| | | TGTATCAGCATATTATTTTTGATGATGATACCGAAATTAAAACC |
| | | AGCATTAACCATCCGTTTGGCAAAGATAAAATTCTGGCGCGTGA |
| | | TGTGAAAGTGGGCGATTATCTGAACAGCAAAAAAGTGCTGTATA |
| | | ACGAACTGGTGAACGAAAACATTTTTCTGTATGATCCGATTAAC |
| | | GTGGAAAAAGAAAGCCTGTATATTACCAACGGCGTGGTGAGCC |
| | | ATAAC |
| 24 | Int^{N} WTAceL-TerL-11 | qtefe |
| | flanking sequence | |
| 25 | Int^{C} WTAceL-TerL-11 | ceflg |
| | flanking sequence | |
| 26 | GS033_TerA-6intein | SISQESYINIEVNGKVETIKIGDLYKKLSFNERKFNEMKLPESVVKN |
| | | NINLKIETPYGFENFYGVNKIKKDKYIHLEFTNGEKLKCSLDHPLSTI |
| | | DGIVKAKDLDKYTEVYTKFGGCFLKKSKVINESIELYDIVNSGLKH |
| | | LYYSNNIISHN |
| 27 | Wild type Int^{C} of | MKLPESVVKNNINLKIETPYGFENFYGVNKIKKDKYIHLEFTNGEK |
| | GS033_TerA-6 | LKCSLDHPLSTIDGIVKAKDLDKYTEVYTKFGGCFLKKSKVINESIE |
| | | LYDIVNSGLKHLYYSNNIISHN |
| 28 | Wild type Int^{N} of | SISQESYINIEVNGKVETIKIGDLYKKLSFNERKFNE |
| | GS033_TerA-6 | |
| 29 | Int^{N} of GS033_TerA-6 | CISQESYINIEVNGKVETIKIGDLYKKLSFNERKFNE |
| | with S1C substitution | |
| 30 | Int^{N} of GS033_TerA-6 (minus | SISQESYINIEVNGKVETIKIGDLYKKL |
| | 9 aa) | |
| 31 | Int^{N} of GS033_TerA-6 (minus | SISQESYINIEVNGKVETIKIGDLYKKL |
| | 9 aa synthetic) | |
| 32 | Int^{N} of GS033_TerA-6 (minus | SISQESYINIEVNGKVETIKIGDLYKKLSFNERK |
| | 3 aa synthetic) | |
| 33 | Int^{N} AceL-TerL-11 with C1S | SVSGDTMVETEDGKIKIEDLYKRLA |
| | and Y3S substitutions | |
| | (synthetic) | |
| 34 | Int^{N} GS033_TerA-6 flanking | kvefe |
| | sequence | |
| 35 | Int^{C} GS033_TerA-6 flanking | ceflg |
| | sequence | |
| 36 | DNA Int^{N} GS033_TerA-6 | AAGGTTGAGTTTGAGTCAATTTCTCAAGAATCTTACATAAATAT |
| | | CGAAGTTAATGGTAAGGTCGAAACAATTAAAATTGGCGATTTAT |
| | | ATAAAAAACTTTCATTTAACGAAAGAAAATTTAATGAGTGA |
| 37 | DNA Int^{C} GS033_TerA-6 | ATGAAATTACCAGAATCTGTAGTAAAAAACAATATCAACTTAAA |
| | | AATAGAAACTCCATATGGATTTGAGAATTTTTATGGAGTAAATA |
| | | AAATAAAGAAGGATAAGTATATACATTTAGAATTTACCAATGGT |
| | | GAAAAACTAAAGTGCTCTTTAGATCATCCATTATCAACAATTGA |
| | | TGGAATTGTAAAAGCAAAAGATTTAGACAAATATACAGAAGTA |
| | | TATACAAAATTTGGTGGATGCTTTCTAAAAAAATCAAAAGTTAT |
| | | TAATGAATCAATAGAATTATATGATATTGTAAACTCGGGACTAA |
| | | AGCATTTATATTATTCAAATAATATAATATCTCACAACTGCGAA |
| | | TTCTTAGGG |
| 38 | Int^{N} TerA-1_CP21-BP | CCLENTRVQVRNKYTNKIETLTIKELYARLQELKKS |
| 39 | Int^{C} TerA-1_CP21-BP | MSEIQDINPYEILTPQGFKPFVDIIKSIQTTGITITLEDSREISVTLDHK |
| | | FKHLNDYKEAKYFKVGDKLQCSKIIKIENIEGEFYEPLEVQDHEYIA |
| | | NDFINHN |
| 40 | Int^{N} TerA-1_CP21-BP | frgfs |
| | flanking sequence | |
| 41 | Int^{C} TerA-1_CP21-BP | cniiv |
| | flanking sequence | |
| 42 | DNA Int^{N} TerA-1_CP21-BP | TTTAGGGGATTCAGTTGCTGTCTCGAAAATACTCGAGTGCAGGT |
| | | AAGAAATAAATATACTAATAAAATAGAAACGCTTACCATAAAG |
| | | GAATTGTATGCTAGGTTACAAGAACTCAAAAAATCTTAA |
| 43 | DNA Int^{C} TerA-1_CP21-BP | GTGAGTGAAATCCAAGATATAAATCCATATGAAATATTAACACC |
| | | ACAAGGATTTAAACCTTTTGTTGATATCATTAAATCAATTCAAA |
| | | CAACTGGCATAACAATAACTTTAGAGGATTCAAGAGAGATATCA |
| | | GTTACATTAGATCACAAATTTAAACACTTAAATGATTATAAAGA |
| | | AGCCAAATATTTTAAAGTAGGTGATAAATTACAGTGTTCAAAAA |
| | | TTATTAAAATTGAAAATATTGAAGGTGAATTTTATGAACCTTTA |
| | | GAAGTTCAAGATCACGAGTATATAGCCAACGACTTTATAAATCA |
| | | TAATTGTAATATAATCGTT |
| 44 | Int^{N} CP81-BP_TerA | CVAGDTKITVRNKKTGVIEDITMEELYNRIG |
| 45 | Int^{C} CP81-BP_TerA | MYEVLTPNGFSDFDDISREKKDVYKVITEDDFIKVTKGHKFETPNG |
| | | FKQLKHLKINDLIKYKNKFSKIVLIDYVGVEYVYDLINVHKNNEYY |
| | | TNNFVSHN |
| 46 | Int^{N} CP81-BP_TerA flanking | ifide |
| | sequence | |
| 47 | Int^{c} CP81-BP_TerA flanking | cafid |
| | sequence | |
| 48 | DNA Int^{N} CP81-BP_TerA | ATTTTTATTGATGAATGTGTAGCTGGTGACACAAAAATTACAGT |
| | | TAGAAATAAGAAAACAGGTGTCATTGAAGATATAACAATGGAA |
| | | GAGTTATATAACAGAATAGGATAA |
| 49 | DNA Int^{C}P81-BP_TerA | ATGTATGAAGTACTAACACCAAATGGATTTAGTGATTTTGATGA |
| | | TATATCAAGAGAAAAAAAAGATGTATATAAAGTAATAACAGAA |
| | | GATGATTTTATAAAAGTAACAAAAGGTCATAAATTTGAAACACC |
| | | TAATGGTTTTAAACAATTAAAACATCTTAAAATTAATGATTTAA |
| | | TAAAATATAAAAATAAATTTTCAAAAATTGTTTTAATAGATTAT |
| | | GTTGGAGTAGAATATGTATATGATTTAATTAATGTACATAAAAA |
| | | TAACGAGTATTATACAAATAATTTTGTTTCACACAATTGTGCGTT |
| | | TATAGAT |
| 50 | Int^{N} AceL-1_ClpC-1 | CFSKKTSIKLRNKKTGDLEEIDISDLIYELHIS |
| 51 | Int^{C} AceL-1_ClpC-1 | MIKLYNKKQNKKFTKSYDLGDYQILTD SGYIGLVSLHETIPYEVWK |
| | | LKLSNGYELECADDHIIFDNEMNEIFVKNLELGDRVKVDDGYAVVI |
| | | ELVNTGLLESMYDFELVEDSNRRYYTNGILSHN |
| 52 | Int^{N} AceL-1_ClpC-1 flanking | sgvgk |
| | sequence | |
| 53 | Int^{C} AceL-1_ClpC-1flanking | telak |
| | sequence | |
| 54 | DNA Int^{N} AceL-1_ClpC-1 | AGCGGGGTTGGTAAATGTTTTTCTAAAAAAACATCAATAAAATT |
| | | AAGGAATAAAAAAACTGGTGATTTAGAAGAAATTGATATTTCTG |
| | | ATCTAATATATGAACTACACATTAGCTAA |
| 55 | DNA Int^{C} AceL-1_ClpC-1 | ATGATAAAATTATATAATAAAAAACAAAATAAAAAATTCACCA |
| | | AATCTTATGATTTGGGTGATTACCAAATACTAACTGATAGTGGA |
| | | TATATTGGTTTGGTCTCATTACATGAGACAATACCATATGAAGTT |
| | | TGGAAATTGAAATTATCTAATGGATATGAATTAGAGTGTGCTGA |
| | | TGATCATATTATTTTTGATAATGAAATGAATGAGATATTTGTAA |
| | | AGAATCTAGAATTAGGAGACAGAGTAAAAGTAGATGATGGATA |
| | | TGCTGTTGTTATAGAATTAGTAAATACTGGTCTATTAGAAAGTA |
| | | TGTATGATTTTGAGTTAGTAGAAGATTCAAATAGAAGGTATTAT |
| | | ACAAATGGTATTTTATCACACAACACAGAACTGGCTAAA |
| 56 | Int^{N} AceL-1 ClpC-2 | CVSPNTKIKIRNSSTGEISEVTIAEFNKMI |
| 57 | Int^{C} AceL-1_ClpC-2 | MKKIVKSVSVEGFEVLSDNGWVPIKNVHTTVPYELYNLRTANGLR |
| | | LECADNHIVFTSKLKEVYVKDLNVDDKIMTEDGVSLVSSIEKTKAK |
| | | VTMYDLEVDSEDHRYYTDGILSHN |
| 58 | Int^{N} AceL-1_ClpC-2 flanking | agvgk |
| | sequence | |
| 59 | Int^{C} AceL-1_ClpC-2 flanking | tslie |
| | sequence | |
| 60 | DNA Int^{N} AceL-1_ClpC-2 | GCAGGAGTAGGTAAATGCGTTAGTCCTAATACGAAGATTAAGAT |
| | | TAGGAACAGTAGCACTGGAGAAATTTCAGAAGTTACGATAGCG |
| | | GAATTCAATAAGATGATTTAA |
| 61 | DNA Int^{C} AceL-1_ClpC-2 | ATGAAAAAAATTGTTAAGAGTGTAAGTGTAGAAGGATTTGAGG |
| | | TACTCTCTGATAATGGATGGGTACCAATTAAAAATGTACATACC |
| | | ACTGTACCCTACGAACTCTATAACCTCCGTACAGCCAACGGTTT |
| | | GCGGTTAGAATGTGCAGACAATCATATCGTGTTTACTTCTAAGC |
| | | TAAAAGAGGTATATGTTAAAGACTTAAATGTTGACGATAAGATT |
| | | ATGACTGAGGATGGAGTATCTTTAGTATCATCAATTGAAAAGAC |
| | | TAAAGCTAAAGTAACGATGTATGATCTTGAAGTAGATAGTGAAG |
| | | ATCATCGTTACTATACTGACGGTATTCTTTCACATAACACTTCTC |
| | | TAATAGAA |
| 62 | Int^{N} AceL-1 RadA1-1 | CVHPNTLVKIKIDSTGEERTITVKDLHELIKSVK |
| 63 | Int^{C} AceL-1_RadA1-1 | MKRKFIESISADNISIMTDTGWEKVKGSHVTIEYKVFNLVTDRLSLQ |
| | | CADDHIVFKEDFSEVFVKDLEVGDLIQTVNGLESVTEVYETDDLVN |
| | | MHDLEIDSKNHRYYTDGILSHN |
| 64 | Int^{N} AceL-1_RadA1-1 | pgvgk |
| | flanking sequence | |
| 65 | Int^{C} AceL-1_RadA1-1 | ttlll |
| | flanking sequence | |
| 66 | DNA Int^{N} AceL-1_RadA1-1 | CCAGGAGTTGGTAAATGCGTCCATCCAAATACATTGGTAAAAAT |
| | | CAAAATTGATTCTACTGGTGAGGAGCGTACTATTACAGTCAAAG |
| | | ACCTCCACGAACTAATTAAATCTGTAAAATGA |
| 67 | DNA Int^{C} AceL-1_RadA1-1 | ATGAAACGTAAATTTATAGAAAGTATTTCTGCAGACAATATCAG |
| | | CATCATGACAGATACTGGTTGGGAAAAAGTTAAAGGTAGTCAC |
| | | GTTACAATTGAGTATAAAGTATTCAACCTTGTCACTGACAGGTT |
| | | ATCACTACAATGTGCAGATGATCATATCGTTTTTAAAGAGGACT |
| | | TCTCAGAGGTCTTTGTAAAGGACCTTGAGGTTGGTGATTTAATA |
| | | CAAACAGTAAACGGTTTAGAATCAGTTACTGAAGTATATGAAAC |
| | | AGACGACTTGGTAAATATGCACGATTTAGAAATTGATTCTAAAA |
| | | ACCATAGGTATTATACTGATGGAATTCTTTCACATAATACTACAT |
| | | TATTATTG |
| 68 | Int^{N} AceL-1_TerL-10 | CVSGDTKVTLKDNDTGKIINVNIEEMVSVSSLDV |
| 69 | Int^{C} AceL-1_TerL-10 | MEVGKMSKSYKVLSPSGFVDFAGIQKITRSKYRHFIFDDGTEIKCSL |
| | | NHRFGEEEIVASTLHHGTELQGKKILYAEDVEDDIDLYDLLNVANG |
| | | NLYYTNGLVSHN |
| 70 | Int^{N} AceL-1_TerL-10 | ntefe |
| | flanking sequence | |
| 71 | Int^{C} AceL-1_TerL-10 | ceflg |
| | flanking sequence | |
| 72 | DNA Int^{N} AceL-1_TerL-10 | AACACGGAGTTTGAGTGTGTTTCTGGTGATACAAAGGTTACTCT |
| | | CAAAGACAATGATACAGGAAAGATTATTAATGTAAATATTGAA |
| | | GAAATGGTGAGTGTGAGTTCTTTGGATGTATAA |
| 73 | DNA Int^{C} AceL-1_TerL-10 | ATGGAAGTTGGAAAGATGTCTAAAAGTTATAAAGTGTTATCACC |
| | | ATCAGGGTTTGTGGATTTTGCTGGTATTCAAAAAATAACACGCA |
| | | GCAAATATCGACATTTTATTTTTGATGATGGCACAGAAATCAAA |
| | | TGTTCGTTAAATCATAGATTTGGTGAAGAGGAAATAGTAGCCTC |
| | | AACACTCCATCACGGCACAGAGCTTCAGGGTAAAAAAATACTGT |
| | | ATGCAGAAGATGTTGAGGATGATATTGATTTATATGATTTGTTA |
| | | AATGTTGCCAATGGAAATCTTTACTACACCAACGGATTAGTATC |
| | | ACACAATTGTGAGTTCCTTGGC |
| 74 | Int^{N} AceL-1_TerL-2 | CFFFNTIISVETNNQQYETRIGILYYSMVSKERNLTILEKIKIKLYDLL |
| | | FILEKH |
| 75 | Int^{C} AceL-1_TerL-2 | MLRIFKRCLIYLIKKMIEFIELYEYKKISLDECDINKKILNSISLMDLK |
| | | VETDTGYETSSNIHITQPFKHYNIETVDGYEIICADNHILFDEEFNEV |
| | | FTKDLKIGDLIKTKNGNSVIKSIYIDTHKS SMFDLTIDHPNHRFYTNG |
| | | ILSHN |
| 76 | Int^{N} AceL-1_TerL-2 flanking | rqvgk |
| | sequence | |
| 77 | Int^{C} AceL-1_TerL-2flanking | tisss |
| | sequence | |
| 78 | DNA Int^{N} AceL-1_TerL-2 | AGGCAAGTTGGAAAATGTTTTTTTTTCAATACAATTATATCCGTT |
| | | GAAACCAATAATCAGCAATATGAAACTAGAATAGGAATTCTTTA |
| | | TTATTCAATGGTTTCAAAGGAAAGAAATTTAACTATTTTAGAGA |
| | | AAATTAAAATAAAATTATATGATTTATTATTCATTTTAGAAAAA |
| | | CATTAA |
| 79 | DNA Int^{C} AceL-1_TerL-2 | ATGCTTAGAATTTTTAAAAGGTGTTTAATTTATTTAATTAAAAAA |
| | | ATGATTGAATTTATTGAATTATATGAATATAAAAAAATCTCATT |
| | | AGATGAGTGTGATATAAATAAAAAAATATTAAACTCAATATCTC |
| | | TTATGGATTTAAAGGTAGAGACTGATACAGGATATGAAACATCA |
| | | TCTAATATACACATAACACAACCATTTAAACACTATAATATTGA |
| | | AACGGTTGATGGTTATGAAATAATATGTGCTGATAATCATATAT |
| | | TATTTGATGAGGAATTCAATGAAGTATTTACAAAGGATTTAAAA |
| | | ATAGGAGATTTAATTAAAACAAAAAATGGCAACAGTGTTATCA |
| | | AGAGTATTTATATAGACACACATAAGTCATCCATGTTTGACCTA |
| | | ACAATAGACCATCCAAACCACAGGTTCTATACAAATGGTATACT |
| | | TTCACATAATACGATATCATCTTCT |
| 80 | Int^{N} AceL-1_TerL-3 | CVDGNTIVETEDGKIKIEDLYKKL |
| 81 | Int^{C} AceL-1_TerL-3 | MFITNTDNIKILSPSGFSNFNGIQKVERNLYQHIIFDDESEIKTSINHPF |
| | | GKNKILARNVKVGDYLSSKKVLYNELVNEKIFLYDPINVEKENLYI |
| | | TNGVVSHN |
| 82 | Int^{N} AceL-1_TerL-3 flanking | qqefe |
| | sequence | |
| 83 | Int^{C} AceL-1_TerL-3 flanking | ceflg |
| | sequence | |
| 84 | DNA Int^{N} AceL-1_TerL-3 | CAACAAGAGTTTGAATGTGTTGACGGTAATACGATAGTCGAAAC |
| | | GGAAGATGGTAAAATAAAAATAGAAGATTTATATAAAAAATTG |
| | | TGA |
| 85 | DNA Int^{C} AceL-1_TerL-3 | ATGTTTATAACTAATACAGATAATATAAAAATATTAAGTCCAAG |
| | | TGGATTTTCTAATTTTAATGGTATTCAAAAGGTTGAAAGAAACC |
| | | TTTATCAACACATTATCTTTGATGATGAATCTGAAATAAAAACTT |
| | | CTATTAACCACCCTTTTGGTAAAAATAAAATATTAGCAAGAAAT |
| | | GTAAAAGTAGGAGATTATTTAAGTAGTAAAAAAGTATTATATAA |
| | | TGAGTTGGTTAATGAAAAAATATTTTTATATGACCCTATAAATG |
| | | TAGAAAAAGAAAACTTATATATTACTAACGGTGTTGTTTCTCAT |
| | | AATTGTGAGTTTTTAGGT |
| 86 | Int^{N} AceL-1_TerL-4 | CVDGNTIVETEDGKIKIEDLYKKM |
| 87 | Int^{C} AceL-1_TerL-4 | MFRTNTDNIKILSPSGFSIFNGIQKVERDLYQHIIFDDKSEIKTSINHPF |
| | | GKDKILARNIKVGDYLNSKKVLYNELVAEKITLYDPINVEKENLYIT |
| | | NGVISHN |
| 88 | Int^{N} AceL-1_TerL4 flanking | qqefe |
| | sequence | |
| 89 | Int^{C} AceL-1_TerL-4 flanking | ceflg |
| | sequence | |
| 90 | DNA Int^{N} AceL-1_TerL-4 | CAACAAGAGTTTGAGTGTGTGGATGGAAATACGATAGTCGAAA |
| | | CGGAAGATGGCAAAATAAAAATAGAAGATTTATATAAAAAAAT |
| | | GTGA |
| 91 | DNA Int^{C} AceL-1_TerL-4 | ATGTTTAGAACAAATACAGATAATATAAAAATTTTAAGTCCAAG |
| | | TGGGTTTTCTATTTTTAATGGCATTCAAAAGGTTGAAAGAGACC |
| | | TCTATCAACATATTATCTTTGATGATAAATCTGAAATAAAGACTT |
| | | CTATCAACCACCCCTTTGGTAAAGATAAAATATTAGCGAGAAAT |
| | | ATAAAGGTTGGTGATTATTTAAATAGTAAGAAAGTTTTATATAA |
| | | TGAGTTGGTCGCCGAAAAGATTACTTTATATGATCCTATAAATG |
| | | TAGAAAAAGAAAATTTATATATCACTAACGGTGTTATTTCTCAT |
| | | AATTGTGAGTTTTTAGGT |
| 92 | Int^{N} AceL-1_TerL-5 | CVDGNTIVETEDGKIKIEDLYKKL |
| 93 | Int^{C} AceL-1_TerL-5 | MFRTNTDNIKILSPSGFSNFNGIQKVERDLYQHIIFDDKSEIKTSINHP |
| | | FGKDKILARNIKVGDYLNSKKVLYNELVNEKITLYDPINVEKENLYI |
| | | TNGVISHN |
| 94 | Int^{N} AceL-1_TerL5 flanking | qqefe |
| | sequence | |
| 95 | Int^{C} AceL-1_TerL-5 flanking | ceflg |
| | sequence | |
| 96 | DNA Int^{N} AceL-1_TerL-5 | CAACAAGAGTTTGAATGTGTTGACGGTAATACGATAGTTGAAAC |
| | | GGAAGATGGTAAAATAAAAATAGAAGATTTATATAAAAAATTA |
| | | TAG |
| 97 | DNA Int^{C} AceL-1_TerL-5 | ATGTTTAGAACCAATACAGATAATATAAAAATATTAAGTCCAAG |
| | | TGGATTTTCTAATTTTAACGGCATTCAAAAGGTTGAAAGAGACC |
| | | TCTATCAACATATTATCTTTGATGATAAGTCTGAAATAAAAACTT |
| | | CTATTAACCACCCTTTTGGTAAAGATAAAATATTAGCGAGAAAT |
| | | ATAAAAGTAGGAGATTATTTAAATAGTAAGAAGGTTTTATATAA |
| | | TGAGTTGGTTAATGAAAAAATTACTTTATATGACCCTATAAATG |
| | | TAGAAAAAGAAAACTTATATATTACTAACGGTGTTATTTCTCAT |
| | | AATTGTGAGTTTTTAGGT |
| 98 | Int^{N} AceL-1_UvsW-3 -1 | CRTYDSTMDIDVGNSDFAEYLLNNSKK |
| 99 | Int^{C} AceL-1_UvsW-3 -1 | MKFNIPIGELAESIAKYKGVLLNDNCEINIKDLDCKVNTPSGTATINI |
| | | IIKKEKLEGIKLLLANGVEIKCANKHILRYNNADVFADSLAIGDSVE |
| | | TINGNVKVSSINNIDDTTFYDIGIDAPYLYYDADGVLHHN |
| 100 | Int^{N} AceL-1_UvsW-3 -1 | tgagk |
| | flanking sequence | |
| 101 | Int^{C} AceL-1_UvsW-3 -1 | titta |
| | flanking sequence | |
| 102 | DNA Int^{N} AceL-1_UvsW-3 -1 | ACTGGTGCAGGCAAATGTCGAACTTATGATTCTACAATGGATAT |
| | | AGATGTAGGTAATTCTGATTTTGCTGAATATTTGCTAAATAATA |
| | | GTAAGAAATAG |
| 103 | DNA Int^{C} AceL-1_UvsW-3 -1 | ATGAAATTTAACATACCAATAGGGGAACTAGCAGAGTCGATCG |
| | | CGAAGTACAAAGGAGTACTATTAAACGATAACTGCGAAATTAA |
| | | TATTAAAGATCTTGATTGTAAAGTTAATACACCATCAGGAACTG |
| | | CTACTATTAATATTATAATTAAAAAAGAAAAGTTAGAAGGCATA |
| | | AAACTATTACTTGCAAATGGTGTAGAAATAAAGTGTGCTAATAA |
| | | GCATATATTAAGATATAATAATGCAGACGTATTTGCAGATTCAT |
| | | TAGCAATTGGCGACTCGGTAGAAACTATTAACGGGAATGTTAAG |
| | | GTTAGTAGTATTAACAATATTGACGATACTACATTTTACGATATC |
| | | GGAATAGATGCACCGTACTTATATTATGATGCAGACGGAGTATT |
| | | ACATCATAATACAATTACCACAGCA |
| 104 | Int^{N} AceL-1 41-1 | CFFSDGEINTRNISNKEIKSIKIGKIFTNISKGHTNI |
| 105 | Int^{C} AceL-1_gp41 -1 | MLDNYEIIEADSLLEGKYDRPLYDKFIEAYEVDNLEVDTPNGWIKIE |
| | | GIGKTIEFYEWEIQTSGGKHLICADKHLLYRCDNMNFYNKKCDITEI |
| | | YCQDLNIGDFIMTKDGPEMLMDIYKNGNKSNMYDLQLSEGSNKQ |
| | | YYTNDILSHN |
| 106 | Int^{N} AceL-1_ 41 -lflanking | slwmq |
| | sequence | |
| 107 | Int^{C} AceL-1_gp41 -lflanking | tnggk |
| | sequence | |
| 108 | DNA Int^{N} AceL-1_gp41 -1 | ACAAACGGAGGAAAATGTTTTTTTAGTGATGGTGAGATAAATAC |
| | | TAGGAATATAAGCAATAAGGAAATAAAATCAATTAAAATAGGT |
| | | AAAATTTTTACCAACATTAGCAAGGGACATACTAACATTTAA |
| 109 | DNA Int^{C} AceL-1_gp41-1 | ATGTTAGATAATTATGAAATAATAGAAGCAGATTCTCTATTAGA |
| | | AGGAAAATATGATAGACCACTATATGATAAATTTATTGAAGCTT |
| | | ATGAAGTAGACAACTTAGAGGTTGATACACCAAATGGTTGGATA |
| | | AAGATAGAAGGAATTGGTAAAACTATTGAATTTTATGAATGGGA |
| | | AATACAAACATCTGGTGGAAAACATCTAATATGTGCAGATAAAC |
| | | ATCTATTATATAGGTGTGATAATATGAATTTTTATAATAAAAAA |
| | | TGTGACATAACAGAAATATACTGCCAAGATTTGAATATAGGTGA |
| | | TTTTATAATGACTAAGGATGGTCCTGAGATGTTGATGGATATTT |
| | | ATAAAAATGGTAATAAATCGAATATGTATGATTTACAATTATCA |
| | | GAAGGCTCTAATAAACAATACTACACAAATGATATACTTAGTCA |
| | | TAATTCACTTTGGATGCAA |
| 110 | Int^{N} AceL-1_gp46-1 | CVDESTLIDVQIIDFEPNLENLEFLDKTDEGKRIFLYIKKSNKSLYEKI |
| | | EKFRKGQ |
| 111 | Int^{C} AceL-1_gp46-1 | MLTLKIGDLYELSKNINILESDIRVSTPGGLKKVFAVDITAKNSDVFS |
| | | IKVNKHELLCSPDHLIRSEDMWVKSKDLKINSVIDTKYGKLTVKEIS |
| | | ILDIKSDLMDLHVDGSEYYTNDIISHN |
| 112 | Int^{N} AceL-1_gp46-1 flanking | ngsgk |
| | sequence | |
| 113 | Int^{C} AceL-1_gp46-1 flanking | sslld |
| | sequence | |
| 114 | DNA Int^{N} AceL-1_gp46-1 | AATGGTTCTGGTAAGTGTGTTGATGAGTCAACACTAATAGATGT |
| | | ACAAATAATTGATTTTGAGCCTAATTTAGAAAATTTAGAATTTTT |
| | | AGACAAAACGGATGAGGGAAAGAGGATTTTTCTATATATAAAG |
| | | AAATCTAATAAATCCTTGTATGAAAAAATTGAAAAATTTAGAAA |
| | | AGGTCAATAA |
| 115 | DNA Int^{C} AceL-1_gp46-1 | ATGTTAACATTAAAGATAGGTGATTTATATGAATTATCAAAAAA |
| | | TATAAATATTTTAGAATCAGACATCCGTGTATCTACCCCAGGTG |
| | | GATTGAAAAAGGTTTTTGCTGTTGATATAACAGCAAAAAATAGC |
| | | GATGTGTTTTCTATAAAAGTTAATAAACATGAACTACTTTGCTCA |
| | | CCAGATCATCTAATAAGATCAGAAGATATGTGGGTTAAATCTAA |
| | | AGATTTAAAAATAAATTCCGTAATAGATACAAAATATGGCAAAC |
| | | TTACTGTTAAGGAGATATCAATTTTGGATATAAAGAGTGATTTG |
| | | ATGGATTTACATGTGGATGGTAGTGAATATTACACTAATGATAT |
| | | AATTAGTCACAACTCATCACTATTAGAT |
| 116 | Int^{N} VidaL_T4Lh-1 | CVHPDTKVTIRRKLC |
| 117 | Int^{C} VidaL_T4Lh-1 | MKELLDLYTEKEINKLLERYTIDQIIDYSQPHVVSVGSIKEEMDSGN |
| | | FIFVDSPDGYVAVSDFVDKGNFEEYRFTYDKKIIRTNEGHLFQTHLG |
| | | WETSKNLYKMYLAGHPIYILHKNGGYKKIDIEKTGNVIPIVDIVVEH |
| | | KNHRYYTDGLSSHN |
| 118 | Int^{N} VidaL_T4Lh-1 flanking | vflas |
| | sequence | |
| 119 | Int^{C} VidaL_T4Lh-1 flanking | tnvgk |
| | sequence | |
| 120 | DNA Int^{N} VidaL_T4Lh-1 | GTATTTTTGGCTAGTTGTGTGCATCCAGATACAAAAGTAACAAT |
| | | TCGTAGAAAACTTTGTTAG |
| 121 | DNA Int^{C} VidaL_T4Lh-1 | ATGAAAGAATTGCTTGACTTATACACAGAAAAAGAAATAAATA |
| | | AATTATTAGAAAGATACACAATAGACCAGATTATAGACTACTCA |
| | | CAACCTCATGTGGTTTCTGTGGGTAGTATAAAAGAAGAAATGGA |
| | | TTCAGGAAATTTCATTTTTGTTGACAGCCCAGATGGTTACGTTGC |
| | | TGTTAGTGATTTTGTAGACAAAGGAAACTTTGAAGAATATAGGT |
| | | TTACATATGATAAAAAAATAATCCGAACAAACGAAGGTCACTTA |
| | | TTCCAAACACATTTGGGTTGGGAGACTTCTAAGAATTTATATAA |
| | | AATGTACTTAGCTGGTCACCCCATTTATATATTGCATAAAAATG |
| | | GTGGTTATAAAAAGATTGATATAGAAAAGACCGGAAACGTGAT |
| | | TCCTATCGTTGATATTGTGGTGGAACACAAAAACCATAGATATT |
| | | ATACGGATGGATTGTCCAGCCATAACACAAATGTGGGCAAG |
| 122 | Int^{N} VidaL_UvsX-2 | CLPKEAVVQIRLTKKG |
| 123 | Int^{C} VidaL_UvsX-2 | MIEEKKVTVQELRELYLSGEYTIEIDTPDGYQTIGKWFDKGVLSMV |
| | | RVATATYETVCAFNHMIQLADNTWVQACELDVGVDIQTAAGIQPV |
| | | MLVEDTSDAECYDFEVMHPNHRYYGDGIVSHN |
| 124 | Int^{N} VidaL_UvsX-2 flanking | sgksy |
| | sequence | |
| 125 | Int^{C} VidaL_UvsX-2 flanking | agesg |
| | sequence | |
| 126 | DNA Int^{N} VidaL_UvsX-2 | GCTGGCGAAAGTGGCTGTTTGCCAAAAGAAGCAGTAGTACAGA |
| | | TTCGATTAACAAAAAAAGGCTAG |
| 127 | DNA Int^{C} VidaL_UvsX-2 | ATGATTGAAGAAAAGAAAGTAACAGTACAAGAGCTTAGAGAGC |
| | | TATATCTCAGCGGCGAGTATACTATTGAGATTGACACACCGGAC |
| | | GGATATCAGACTATCGGAAAATGGTTTGACAAA6GGGTATTGTC |
| | | CATGGTTAGAGTTGCCACAGCCACTTACGAAACAGTGTGTGCAT |
| | | TTAATCATATGATTCAACTGGCTGACAATACGTGGGTACAAGCC |
| | | TGTGAGTTAGATGTAGGAGTAGATATACAAACGGCGGCAGGCA |
| | | TCCAGCCTGTTATGTTAGTCGAAGATACAAGTGATGCAGAGTGT |
| | | TACGATTTTGAAGTCATGCATCCGAATCATAGATATTACGGTGA |
| | | CGGAATTGTAAGCCATAACTCGGGGAAAAGTTAT |
| 128 | Int^{N} VidaL TerL-6-1 | SLAHETIVSINDNNTLTSMCIGDLYDYM |
| 129 | Int^{C} VidaL_TerL-6-1 | MDYHSNQVSRIFGVGMSKVHLGFKKNTKNLKVLTPNGHEEFYGIN |
| | | KIRVDEYIRIKFKEHKEIRCSIDHPFIQENDLPIKAKHIDKSKHIKCID |
| | | GFTTLEYSHVVNKQIELYDIVNSGSEYIYFSNGILSHN |
| 130 | Int^{N} VidaL_TerL-6-1 flanking | sveye |
| | sequence | |
| 131 | Int^{C} VidaL_TerL-6-1 flanking | ckfmg |
| | sequence | |
| 132 | DNA Int^{N} VidaL_TerL-6-1 | TCCGTGGAATATGAGAGTTTGGCACACGAAACTATAGTAAGTAT |
| | | AAATGATAATAACACACTAACAAGTATGTGCATTGGAGATTTAT |
| | | ATGACTATATGTAA |
| 133 | DNA Int^{C} VidaL_TerL-6-1 | TTGGATTACCACTCTAATCAAGTGTCTCGAATTTTTGGAGTTGGT |
| | | ATGAGCAAAGTACATCTAGGGTTTAAAAAGAACACTAAAAATTT |
| | | AAAAGTGTTAACACCAAATGGACACGAAGAATTCTACGGAATA |
| | | AACAAAATACGTGTCGATGAATATATACGAATAAAATTCAAAG |
| | | AACATAAAGAAATACGTTGCTCGATTGACCACCCGTTTATACAA |
| | | GAAAATGATTTACCAATAAAAGCAAAACATATTGATAAAAGCA |
| | | AACATATAAAATGTATTGATGGATTTACTACTTTAGAGTATTCG |
| | | CATGTTGTTAATAAACAAATTGAACTATATGATATTGTAAACTC |
| | | TGGTAGTGAGTACATATATTTTTCTAATGGGATATTAAGTCACA |
| | | ACTGTAAATTCATGGGT |
| 134 | Int^{N} TerL-7-1 VidaL | CLWGASTVNVFDSLTGKNIDIKLEDLYQKL |
| 135 | Int^{C} TerL-7-1_VidaL | MESYTFRKNTRYKIMTPAGYQNFGGIRKLNKNVHYIVELSNKKILK |
| | | CSTTHPFIYNDREIFANKLKVGSLLDSTSKKKISVISIELDKSKIDLYD |
| | | IVEVNNGNIFNVDGIVSHN |
| 136 | Int^{N} TerL-7-1_VidaL flanking | sqecd |
| | sequence | |
| 137 | Int^{C} TerL-7-1_VidaL flanking | c |
| | sequence | |
| 138 | DNA Int^{N} TerL-7-1_VidaL | TCCCAAGAATGCGATTGTTTGTGGGGCGCATCTACTGTAAATGT |
| | | ATTTGATAGTTTAACTGGAAAAAACATTGATATAAAACTCGAAG |
| | | ATTTGTATCAAAAACTTTAA |
| 139 | DNA Int^{C} TerL-7-1_VidaL | ATGGAATCATATACTTTTAGAAAAAACACAAGATATAAAATAAT |
| | | GACACCAGCAGGATATCAAAACTTTGGTGGTATTAGAAAATTGA |
| | | ATAAAAATGTACATTATATAGTTGAATTATCCAATAAAAAAATA |
| | | TTAAAATGTTCAACTACACATCCATTTATTTATAATGATAGAGA |
| | | GATATTTGCAAATAAATTAAAAGTCGGTAGTTTACTTGATAGTA |
| | | CTAGTAAAAAGAAAATTTCAGTAATATCAATTGAATTAGATAAA |
| | | TCAAAAATAGATTTATATGATATAGTAGAAGTAAATAATGGTAA |
| | | TATTTTTAATGTAGATGGTATTGTTTCACATAATTGT |
| 140 | Int^{N} VidaL_TerL-3 | CVSASTIITLQDTHGNIFDSQIGDLYNTIGK |
| 141 | Int^{C} VidaL_TerL-3 | MSKIFKENTNGYKVLTPAGFQDFAGVSMMGIKPLLRLEFERGAYV |
| | | ECTYDHKFYIDLETCKPAQDIAVGNTVVTSEGDIKLLNKIELGYSEP |
| | | VYDLIQVEGGHRYYTNKILSSN |
| 142 | Int^{N} VidaL_TerL-3 flanking | rreyg |
| | sequence | |
| 143 | Int^{C} VidaL_TerL-3 flanking | ceflv |
| | sequence | |
| 144 | DNA Int^{N} VidaL_TerL-3 | CGTCGTGAGTACGGTTGTGTGAGCGCATCTACAATCATTACTCT |
| | | CCAAGACACACACGGTAATATATTTGACTCACAAATAGGCGACT |
| | | TGTACAATACGATAGGTAAATAA |
| 145 | DNA Int^{C} VidaL_TerL-3 | ATGAGCAAGATTTTTAAAGAGAATACTAATGGATATAAGGTGTT |
| | | AACACCAGCGGGGTTTCAAGACTTTGCTGGTGTTAGCATGATGG |
| | | GAATAAAACCGTTGCTTCGGCTAGAGTTCGAGCGAGGCGCCTAC |
| | | GTCGAATGCACCTACGATCATAAATTTTACATAGACCTAGAAAC |
| | | TTGTAAGCCAGCCCAAGACATTGCAGTAGGAAACACTGTGGTTA |
| | | CTTCTGAGGGTGATATAAAATTACTCAACAAAATAGAACTGGGT |
| | | TATTCAGAACCTGTTTATGATCTTATACAAGTTGAAGGCGGCCA |
| | | CCGATATTACACAAACAAAATACTCAGCTCAAATTGCGAATTTT |
| | | TAGTA |
| 146 | Int^{N} VidaL_TerL-1 | CVQADTKYTIRNKISGDVLNVTAEEFHKMQKK |
| 147 | Int^{C} VidaL_TerL-1 | MKLSNFTNRKFIETIDASEWEVETCEGFKPIISSNKTIEYVVYKIELE |
| | | NGLSIKCADTHILIDKNLQEIYAKDSFNKIIFTKFGNSKVISVETLNIS |
| | | ENMYDLSVDSEDHTYYTDDILSHN |
| 148 | Int^{N} VidaL_TerL-1 flanking | rqqgk |
| | sequence | |
| 149 | Int^{C} VidaL_TerL-1 flanking | tttaa |
| | sequence | |
| 150 | DNA Int^{N} VidaL_TerL-1 | AGACAACAAGGTAAGTGCGTTCAAGCAGACACTAAATACACTA |
| | | TAAGAAACAAAATTAGTGGTGATGTGTTAAATGTTACAGCAGAA |
| | | GAATTCCACAAAATGCAGAAAAAATAA |
| 151 | DNA Int^{C} VidaL_TerL-1 | ATGAAGCTATCCAATTTCACCAATAGAAAATTTATAGAAACAAT |
| | | TGATGCTAGTGAATGGGAAGTAGAAACATGCGAAGGTTTCAAA |
| | | CCCATCATTAGTTCAAATAAAACTATTGAATATGTAGTCTATAA |
| | | AATTGAACTAGAAAATGGATTATCTATTAAATGTGCAGACACTC |
| | | ATATTTTAATAGATAAAAATTTGCAAGAAATTTATGCAAAAGAT |
| | | AGTTTTAATAAAATAATATTTACAAAGTTCGGAAACTCAAAAGT |
| | | TATTTCCGTAGAAACTTTAAATATATCTGAAAATATGTATGATCT |
| | | TTCTGTTGATTCAGAAGATCACACATACTATACAGATGATATCTT |
| | | ATCACATAATACCACGACCGCCGCA |
| 152 | Int^{N} VidaL_gp46-1 | CLCINTIVKVKNTKTGVIYETTIGELYNGAME |
| 153 | Int^{C} VidaL_gp46-1 | MSTISQTVNRKFVNSFSLVDLEIETDSGWQPVTDIHKTIPYTVWHIE |
| | | TQSGLTLDCADTHILFDHNYNEIFVKDIIPNQTKIISKHGPELVLTVIE |
| | | QSQQENMFDLTVDHPDHRFYSNNILSHN |
| 154 | Int^{N} VidaL_gp46-1 flanking | ngtgk |
| | sequence | |
| 155 | Int^{C} VidaL_gp46-1 flanking | ttvin |
| | sequence | |
| 156 | DNA Int^{N} VidaL_gp46-1 | AACGGAACGGGCAAGTGCCTTTGTATAAATACTATTGTAAAAGT |
| | | AAAAAACACCAAAACTGGGGTAATTTACGAAACTACAATAGGA |
| | | GAATTATACAATGGCGCGATGGAATAA |
| 157 | DNA Int^{C} VidaL_gp46-1 | ATGTCTACAATTTCTCAAACAGTAAATAGAAAATTTGTTAATAG |
| | | TTTCAGCTTAGTTGATCTTGAAATCGAAACAGATTCTGGATGGC |
| | | AGCCTGTTACTGACATACACAAGACTATTCCATACACAGTTTGG |
| | | CATATCGAAACCCAAAGCGGGCTGACTCTTGACTGTGCCGACAC |
| | | TCATATCTTGTTTGATCACAACTACAACGAGATCTTTGTCAAAG |
| | | ATATAATACCTAACCAGACTAAGATAATATCTAAGCACGGTCCT |
| | | GAATTAGTATTAACAGTAATCGAACAGTCTCAGCAAGAAAATAT |
| | | GTTTGATCTAACAGTTGATCATCCTGATCATCGTTTTTACTCAAA |
| | | CAATATCTTATCTCACAATACCACAGTGATAAAT |
| 158 | Int^{N} VidaL_gp41-1 | CVIAETEVKIIELYNIDSFIKTGILNSQGVVSWEETSSHGRTR |
| 159 | Int^{C} VidaL_gp41-1 | MNLLDKRVEWLSKWYPVDKLQQLSEDKLVLLYNNSQPKKVRMG |
| | | SLEGVPTSSYRISSPDGYVTAHAWRNKGTKECVTLTTDSGNSITAST |
| | | DHFFEMSDGKWKYAGCLFPGQCISTESGTETVTSVVAAGKHTVYD |
| | | FYIDHENHRYYTNGISSHN |
| 160 | Int^{N} VidaL_gp41-1 flanking | ifagg |
| | sequence | |
| 161 | Int^{C} VidaL_gp41-1 flanking | sgagk |
| | sequence | |
| 162 | DNA Int^{N} VidaL_gp41-1 | ATATTTGCCGGCGGATGTGTAATTGCCGAAACTGAGGTAAAAAT |
| | | AATTGAATTATACAACATTGACAGCTTTATTAAAACAGGAATAC |
| | | TTAACAGCCAAGGGGTCGTTTCGTGGGAAGAAACTTCTTCCCAC |
| | | GGTCGTACTCGATAG |
| 163 | DNA Int^{C} VidaL_gp41-1 | ATGAATTTACTAGATAAGAGAGTTGAGTGGTTATCTAAGTGGTA |
| | | CCCAGTAGACAAGTTACAACAATTATCAGAAGACAAGTTGGTAC |
| | | TCCTATATAACAATAGTCAGCCAAAAAAAGTCCGCATGGGATCG |
| | | TTGGAAGGTGTTCCGACCAGCAGTTATCGGATCAGCAGCCCCGA |
| | | CGGGTATGTTACCGCACATGCCTGGCGAAATAAAGGAACTAAA |
| | | GAGTGTGTTACTCTAACCACAGACTCTGGAAATTCTATAACTGC |
| | | TAGCACAGATCATTTTTTCGAAATGTCCGACGGCAAGTGGAAAT |
| | | ATGCAGGCTGTTTGTTTCCAGGACAGTGCATTAGCACAGAATCC |
| | | GGCACCGAAACAGTGACCAGCGTAGTAGCAGCAGGTAAGCACA |
| | | CAGTATACGACTTCTACATCGATCATGAAAATCACAGATACTAT |
| | | ACCAATGGAATAAGCAGTCATAACTCTGGTGCAGGCAAA |
| 164 | Int^{N} GS013_ter-3 | CLGGDTEIEILDDNGIVQKTSMENLYERL |
| 165 | Int^{C} GS013_ter-3 | MFKINKNIKVKTPDGFKDFSGIQKVYKPFYHWIIFDDGSEIKCSDNH |
| | | SFGKEKIKASTIKVDDILQEKKVLYNEIVEEGIYLYDLLDVGEDNLY |
| | | YSNNIVSHN |
| 166 | Int^{N} GS013_ter-3 flanking | rvefe |
| | sequence | |
| 167 | Int^{C} GS013_ter-3 flanking | ceflg |
| | sequence | |
| 168 | DNA Int^{N} GS013_ter-3 | CGGGTTGAGTTTGAATGTTTGGGTGGTGATACAGAGATTGAAAT |
| | | TTTGGATGATAATGGAATTGTACAAAAAACTTCTATGGAAAATT |
| | | TATATGAACGATTGTGA |
| 169 | DNA Int^{C} GS013_ter-3 | ATGTTTAAGATTAATAAAAATATTAAAGTAAAAACACCTGATGG |
| | | ATTTAAAGATTTTTCAGGAATACAAAAAGTTTATAAACCTTTTTA |
| | | CCATTGGATAATATTTGATGACGGATCAGAAATAAAATGCTCCG |
| | | ATAATCATTCTTTCGGAAAAGAAAAAATTAAGGCATCAACAATT |
| | | AAAGTTGATGATATTTTACAAGAAAAGAAAGTATTATATAATGA |
| | | AATAGTAGAAGAAGGAATTTATCTTTATGATTTACTTGATGTTG |
| | | GCGAAGACAaTCTTTACTATTCAAACAATATAGTATCACACAAC |
| | | TGCGAGTTCTTGGGT |
| 170 | Int^{N} GS013_ter-2 | CLGGDTEIEILDDNGIVQKTSMENLYERL |
| 171 | Int^{C} GS013_ter-2 | MSVGKMFKINKNIKVKTPDGFKDFSGIQKVYKPFYHWIIFDDGSEIK |
| | | CSDNHSFGKEKIKASTIKVDDILQEKKVLYNEIVEEGIYLYDLLDVG |
| | | EDNLYYSNNIVSHN |
| 171 | Int^{N} GS013_ter-2 flanking | rvefe |
| | sequence | |
| 173 | Int^{C} GS013_ter-2 flanking | ceflg |
| | sequence | |
| 174 | DNA Int^{N} GS013_ter-2 | CGTGTTGAGTTTGAATGTTTGGGTGGTGATACAGAGATTGAAAT |
| | | TTTGGATGATAATGGAATAGTACAAAAAACTTCTATGGAAAATT |
| | | TATATGAACGATTGTGA |
| 175 | DNA I Int^{C} GS013_ter-2 | ATGAGTGTTGGAAAAATGTTTAAGATTAATAAAAATATTAAAGT |
| | | AAAAACACCTGATGGATTTAAAGATTTTTCAGGAATACAAAAAG |
| | | TTTATAAACCTTTTTACCATTGGATAATATTTGATGACGGATCAG |
| | | AAATAAAATGCTCCGATAATCATTCTTTCGGAAAAGAAAAAATT |
| | | AAGGCATCAACAATTAAAGTTGATGATATTTTACAAGAAAAGA |
| | | AAGTATTATATAATGAAATAGTAGAAGAAGGAATTTATCTTTAT |
| | | GATTTACTTGATGTTGGCGAAGACAATCTTTACTATTCAAACAA |
| | | TATAGTATCACACAACTGCGAATTCTTAGGT |
| 176 | Int^{N} GS013_ter-1 | CFNTNTTVRLRNKLTGEIIEVTIGEFYEKIKKESNTDLP |
| 177 | Int^{C} GS013_ter-1 | MSKFIEEXXTDEWEVETPSGWQSFSGVGKTIEYEEWEVVTETGKSL |
| | | ICADKHILLNDKWQEVYCEDCSIDDCIQTKNXAEKILQLKKTSRIXN |
| | | MYDLLDVDNGNIFYSNEIVSHN |
| 178 | Int^{N} GS013_ter-1 flanking | rqtgk |
| | sequence | |
| 179 | Int^{C} GS013_ter-1 flanking | sttvv |
| | sequence | |
| 180 | DNA Int^{N} GS013_ter-1 | CGTCAGACGGGTAAATGTTTTAATACAAATACAACGGTAAGGTT |
| | | AAGGAATAAACTTACTGGAGAAATTATTGAAGTGACTATTGGAG |
| | | AATTTTATGAAAAAATCAAGAAAGAAAGTAATACTGATTTGCCT |
| | | TGA |
| 181 | DNA Int^{C} GS013_ter-1 | ATGTCTAAATTTATTGAAGAArTAmAAACTGATGAATGGGAAGT |
| | | AGAAACTCCTTCTGGATGGCAATCTTTTTCTGGGGTAGGAAAAA |
| | | CTATAGAATATGAAGAATGGGAGGTTGTAACCGAAACTGGAAA |
| | | ATCTCTTATATGTGCAGATAAACACATCTTATTAAATGATAAAT |
| | | GGCAAGAAGTTTATTGTGArGATTGTTCCATTGATGACTGTATAC |
| | | AAACAAAAAATkGCGCAGAAAAAATATTACaATTAAAAAAAAC |
| | | ATCAAGAATTyyTAATATGTATGATCTTCTTGATGTTGATAATGG |
| | | TAATATATTTTACAGTAATGAAATAGTTTCACACAATTCTACAA |
| | | CTGTTGTC |
| 182 | Int^{N} GS020_ter-7 | CVDGSSIITIKNKETNLIEKITIEELYNKLL |
| 183 | Int^{C} GS020_ter-7 | MKTNTKYEILGPEGFVDFKGIQKLKKKTRQIFFECGLTLRASYNHKI |
| | | YDYFGDEIIIKDVVIGSKIKSHNGYLIVNSIKDFDYESDVYDVIDSGD |
| | | SHLYYTNNIVSHN |
| 184 | Int^{N} GS020_ter-7 flanking | sqele |
| | sequence | |
| 185 | Int^{C} GS020_ter-7 flanking | cnflg |
| | sequence | |
| 186 | DNA Int^{N} GS020_ter-7 | TCGCAgGAATTAGAgtGTGtTGATGGTTCCTCAatTATaACTATAAA |
| | | AAACAAAGAGACAAATTTAATAGAAAAAATAACAATAGAAGAA |
| | | TTATACAATAAATTGTTATAG |
| 187 | DNA Int^{C} GS020_ter-7 | ATGAAAACTAACACAAAATATGAAATTTTAGGTCCTGAAGGATT |
| | | CGTCGATTTCAAAGGTATTCAAAAATTAAAAAAGAAAACTAGA |
| | | CAAATTTTTTTTGAGTGTGGACTAACATTACGAGCAAGTTATAA |
| | | CCACAAGATTTACGATTATTTTGGGGATGAAATTATAATTAAAG |
| | | ACGTAGTTATTGGTAGTAAAATCAAATCACATAATGGTTATTTA |
| | | ATTGTTAATAGTATCAAGGATTTTGATTATGAAAGTGACGTATA |
| | | TGACGTTATTGATTCAGGTGATTCACATTTATACTACACAAACA |
| | | ACATTGTTTCTCATAATTGTAATTTTCTTGGG |
| 188 | Int^{N} WT AceL-TerL-11 | TGTGTTTATGGTGATACAATGGTTGAAACAGAAGATGGTAAAAT |
| | original DNA sequence | AAAAATAGAAGATTTATATAAAAGGTTGGCA |
| 189 | Int^{C} WT AceL-TerL-11 | ATGTTTAGAACTAATACAAATAATATAAAAATATTAAGTCCAAA |
| | original DNA sequence | TGGATTTTCTAATTTTAATGGTATTCAAAAGGTTGAAAGAAACC |
| | | TTTATCAACACATTATCTTTGATGATGATACTGAAATAAAAACTT |
| | | CCATTAATCATCCTTTTGGTAAAGATAAAATATTAGCAAGAGAT |
| | | GTAAAAGTAGGAGATTATTTAAATAGTAAAAAGGTATTATATAA |
| | | TGAGTTGGTTAATGAAAATATATTTTTATATGATCCTATAAATGT |
| | | AGAAAAAGAAAGTTTATATATTACTAATGGTGTTGTTTCTCATA |
| | | ATTGT |
| 190 | Int^{N} WT AceL-TerL-11 | TGCGTGTATGGCGATACTATGGTGGAAACCGAAGATGGCAAAA |
| | codon optimised DNA | TTAAAATTGAAGATCTGTATAAACGTCTGGCC |
| | sequence | |
| 191 | Int^{C} WT AceL-TerL-11 | GGCATGTTTCGTACCAACACCAACAACATTAAAATTCTGAGCCC |
| | codon optimised DNA | GAACGGCTTTAGCAACTTTAACGGCATTCAGAAAGTGGAACGTA |
| | sequence | ACCTGTATCAGCATATTATTTTTGATGATGATACCGAAATTAAA |
| | | ACCAGCATTAACCATCCGTTTGGCAAAGATAAAATTCTGGCGCG |
| | | TGATGTGAAAGTGGGCGATTATCTGAACAGCAAAAAAGTGCTGT |
| | | ATAACGAACTGGTGAACGAAAACATTTTTCTGTATGATCCGATT |
| | | AACGTGGAAAAAGAAAGCCTGTATATTACCAACGGCGTGGTGA |
| | | GCCATAAC |
| 192 | DNA Int^{N} GS033_TerA-6 | AGCATTAGCCAGGAATCCTATATCAACATTGAGGTGAACGGGA |
| | codon optimised DNA | AAGTGGAAACCATCAAAATCGGCGACCTGTATAAAAAACTGTC |
| | sequence | CTTCAACGAGCGTAAATTCAACGAG |
| 193 | DNA Int^{C} GS033_TerA-6 | ATGAAACTGCCGGAGAGCGTGGTGAAAAACAACATCAACCTGA |
| | codon optimised DNA | AAATCGAAACCCCGTATGGCTTTGAGAACTTCTATGGTGTGAAC |
| | sequence | AAAATCAAAAAAGACAAATATATCCACCTGGAGTTTACCAACG |
| | | GCGAAAAACTGAAATGCTCCCTGGATCATCCTCTGTCTACCATT |
| | | GACGGCATCGTTAAAGCGAAAGATCTGGACAAATATACCGAGG |
| | | TCTATACGAAATTTGGTGGCTGCTTTCTGAAAAAATCCAAAGTG |
| | | ATCAACGAGTCCATCGAGCTGTATGATATCGTGAACTCTGGGCT |
| | | GAAACACCTGTATTATTCCAACAATATTATCAGTCACAAC |
| 194 | Int^{C} | MFITNTDNIKILSPSGFSNFNGIQKVERNLYQHIIFDDESEIKTSINHPF |
| | WTAceL-TerL-3 | GKNKILARNVKVGDYLSSKKVLYNELVNEKIFLYDPINVEKENLYI |
| | | TNGVVSHN |
| 195 | Int^{C} | MFRTNTDNIKILSPSGFSIFNGIQKVERDLYQHIIFDDKSEIKTSINHPF |
| | WTAceL-TerL-4 | GKDKILARNIKVGDYLNSKKVLYNELVAEKITLYDPINVEKENLYIT |
| | | NGVISHN |
| 196 | Int^{C} | MFRTNTDNIKILSPSGFSNFNGIQKVERDLYQHIIFDDKSEIKTSINHP |
| | WTAceL-TerL-5 | FGKDKILARNIKVGDYLNSKKVLYNELVNEKITLYDPINVEKENLYI |
| | | TNGVISHN |
| 197 | Amino acid sequence SBP- | MDEKTTGWRGGHVVEGLAGELEQLRARLEHHPQGQREPGASGGG |
| | (VidaL_T4Lh-1)^{C}-Trx-His₆ | GSSSNNNNNNNNNNLGIEGRISEFKELLDLYTEKEINKLLERYTIDQI |
| | | IDYSQPHVVSVGSIKEEMDSGNFIFVDSPDGYVAVSDFVDKGNFEE |
| | | YRFTYDKKIIRTNEGHLFQTHLGWETSKNLYKMYLAGHPIYILHKN |
| | | GGYKKIDIEKTGNVIPIVDIVVEHKNHRYYTDGLSSHNTNVGGSGG |
| | | TGMSDKIIHLTDDSFDTDVLKADGAILVDFWAEWCGPCKMIAPILD |
| | | EIADEYQGKLTVAKLNIDQNPGTAPKYGIRGIPTLLLFKNGEVAAT |
| | | KVGALSKGQLKEFLDANLAGSVDRSHHHHHH |
| 198 | DNA sequence of SBP- | ATGGGCACTAGTAAAGAACTGCTGGATCTGTATACCGAAAAAG |
| | (VidaL_T4Lh-1)^{C}-Trx-His₆ | AAATTAACAAACTGCTGGAACGCTATACCATTGATCAGATTATT |
| | encoding gene | GATTATAGCCAGCCGCATGTGGTGAGCGTGGGCAGCATTAAAG |
| | | AAGAAATGGATAGCGGCAACTTTATTTTTGTGGATAGCCCGGAT |
| | | GGCTATGTGGCGGTGAGCGATTTTGTGGATAAAGGCAACTTTGA |
| | | AGAATATCGCTTTACCTATGATAAAAAAATTATTCGCACCAACG |
| | | AAGGCCATCTGTTTCAGACCCATCTGGGCTGGGAAACCAGCAAA |
| | | AACCTGTATAAAATGTATCTGGCGGGCCATCCGATTTATATTCT |
| | | GCATAAAAACGGCGGCTATAAAAAAATTGATATTGAAAAAACC |
| | | GGCAACGTGATTCCGATTGTGGATATTGTGGTGGAACATAAAAA |
| | | CCATCGCTATTATACCGATGGCCTGAGCAGCCATAACACCAACG |
| | | TGGGCGGCAGCGGCGGTACCGGTATGAGCGATAAAATTATTCAC |
| | | CTGACTGACGACAGTTTTGACACGGATGTACTCAAAGCGGACGG |
| | | GGCGATCCTCGTCGATTTCTGGGCAGAGTGGTGCGGTCCGTGCA |
| | | AAATGATCGCCCCGATTCTGGATGAAATCGCTGACGAATATCAG |
| | | GGCAAACTGACCGTTGCAAAACTGAACATCGATCAAAACCCTG |
| | | GCACTGCGCCGAAATATGGCATCCGTGGTATCCCGACTCTGCTG |
| | | CTGTTCAAAAACGGTGAAGTGGCGGCAACCAAAGTGGGTGCAC |
| | | TGTCTAAAGGTCAGTTGAAAGAGTTCCTCGACGCTAACCTGGCC |
| | | GGCTCTGTCGACAGATCTCATCACCATCACCATCACTAA |
| 199 | (VidaL_T4Lh-1)^{N} peptide | LASCVHPDTKVTIRRKLC |
| 200 | Amino acid sequence | MGSTKELLDLYTEKEINKLLERYTIDQIIDYSQPHVVSVGSIKEEMD |
| | (VidaL_T4Lh-1)^{C}-Trx-His₆ | SGNFIFVDSPDGYVAVSDFVDKGNFEEYRFTYDKKIIRTNEGHLFQT |
| | (co-expression experiment) | HLGWETSKNLYKMYLAGHPIYILHKNGGYKKIDIEKTGNVIPIVDIV |
| | | VEHKNHRYYTDGLSSHNTNVGGSGGTGMSDKIIHLTDDSFDTDVL |
| | | KADGAILVDFWAEWCGPCKMIAPILDEIADEYQGKLTVAKLNIDQ |
| | | NPGTAPKYGIRGIPTLLLFKNGEVAATKVGALSKGQLKEFLDANLA |
| | | GSVDRSHHHHHH |
| 201 | DNA sequence of gene | ATGGGCACTAGTAAAGAACTGCTGGATCTGTATACCGAAAAAG |
| | fragment encoding | AAATTAACAAACTGCTGGAACGCTATACCATTGATCAGATTATT |
| | (VidaL_T4Lh-1)^{C}-Trx-His₆ | GATTATAGCCAGCCGCATGTGGTGAGCGTGGGCAGCATTAAAG |
| | (co expressionexperiment) | AAGAAATGGATAGCGGCAACTTTATTTTTGTGGATAGCCCGGAT |
| | | GGCTATGTGGCGGTGAGCGATTTTGTGGATAAAGGCAACTTTGA |
| | | AGAATATCGCTTTACCTATGATAAAAAAATTATTCGCACCAACG |
| | | AAGGCCATCTGTTTCAGACCCATCTGGGCTGGGAAACCAGCAAA |
| | | AACCTGTATAAAATGTATCTGGCGGGCCATCCGATTTATATTCT |
| | | GCATAAAAACGGCGGCTATAAAAAAATTGATATTGAAAAAACC |
| | | GGCAACGTGATTCCGATTGTGGATATTGTGGTGGAACATAAAAA |
| | | CCATCGCTATTATACCGATGGCCTGAGCAGCCATAACACCAACG |
| | | TGGGCGGCAGCGGCGGTACCGGTATGAGCGATAAAATTATTCAC |
| | | CTGACTGACGACAGTTTTGACACGGATGTACTCAAAGCGGACGG |
| | | GGCGATCCTCGTCGATTTCTGGGCAGAGTGGTGCGGTCCGTGCA |
| | | AAATGATCGCCCCGATTCTGGATGAAATCGCTGACGAATATCAG |
| | | GGCAAACTGACCGTTGCAAAACTGAACATCGATCAAAACCCTG |
| | | GCACTGCGCCGAAATATGGCATCCGTGGTATCCCGACTCTGCTG |
| | | CTGTTCAAAAACGGTGAAGTGGCGGCAACCAAAGTGGGTGCAC |
| | | TGTCTAAAGGTCAGTTGAAAGAGTTCCTCGACGCTAACCTGGCC |
| | | GGCTCTGTCGACAGATCTCATCACCATCACCATCACTAA |
| 202 | Amino acid sequence MBP- | MGTKTEEGKLVIWINGDKGYNGLAEVGKKFEKDTGIKVTVEHPDK |
| | (VidaL_T4Lh-1)^{N}-linker-SBP | LEEKFPQVAATGDGPDIIFWAHDRFGGYAQSGLLAEITPDKAFQDK |
| | (co-expression experiment) | LYPFTWDAVRYNGKLIAYPIAVEALSLIYNKDLLPNPPKTWEEIPAL |
| | | DKELKAKGKSALMFNLQEPYFTWPLIAADGGYAFKYENGKYDIKD |
| | | VGVDNAGAKAGLTFLVDLIKNKHMNADTDYSIAEAAFNKGETAM |
| | | TINGPWAWSNIDTSKVNYGVTVLPTFKGQPSKPFVGVLSAGINAAS |
| | | PNKELAKEFLENYLLTDEGLEAVNKDKPLGAVALKSYEEELAKDP |
| | | RIAATMENAQKGEIMPNIPQMSAFWYAVRTAVINAASGRQTVDEA |
| | | LKDAQTNSSSNNNNNNNNNNLGIEGRGTLELASCVHPDTKVTIRRK |
| | | LCSEFGSPRKVIKMESEERSMDEKTTGWRGGHVVEGLAGELEQLR |
| | | ARLEHHPQGQREP |
| 203 | DNA sequence of gene | ATGGGTACCAAAACTGAAGAAGGTAAACTGGTAATCTGGATTA |
| | fragment encoding MBP- | ACGGCGATAAAGGCTATAACGGTCTCGCTGAAGTCGGTAAGAA |
| | (VidaL_T4Lh-1)^{N}-linker-SBP | ATTCGAGAAAGATACCGGAATTAAAGTCACCGTTGAGCATCCGG |
| | (co-expression experiment) | ATAAACTGGAAGAGAAATTCCCACAGGTTGCGGCAACTGGCGA |
| | | TGGCCCTGACATTATCTTCTGGGCACACGACCGCTTTGGTGGCT |
| | | ACGCTCAATCTGGCCTGTTGGCTGAAATCACCCCGGACAAAGCG |
| | | TTCCAGGACAAGCTGTATCCGTTTACCTGGGATGCCGTACGTTA |
| | | CAACGGCAAGCTGATTGCTTACCCGATCGCTGTTGAAGCGTTAT |
| | | CGCTGATTTATAACAAAGATCTGCTGCCGAACCCGCCAAAAACC |
| | | TGGGAAGAGATCCCGGCGCTGGATAAAGAACTGAAAGCGAAAG |
| | | GTAAGAGCGCGCTGATGTTCAACCTGCAAGAACCGTACTTCACC |
| | | TGGCCGCTGATTGCTGCTGACGGGGGTTATGCGTTCAAGTATGA |
| | | AAACGGCAAGTACGACATTAAAGACGTGGGCGTGGATAACGCT |
| | | GGCGCGAAAGCGGGTCTGACCTTCCTGGTTGACCTGATTAAAAA |
| | | CAAACACATGAATGCAGACACCGATTACTCCATCGCAGAAGCTG |
| | | CCTTTAATAAAGGCGAAACAGCGATGACCATCAACGGCCCGTG |
| | | GGCATGGTCCAACATCGACACCAGCAAAGTGAATTATGGTGTAA |
| | | CGGTACTGCCGACCTTCAAGGGTCAACCATCCAAACCGTTCGTT |
| | | GGCGTGCTGAGCGCAGGTATTAACGCCGCCAGTCCGAACAAAG |
| | | AGCTGGCAAAAGAGTTCCTCGAAAACTATCTGCTGACTGATGAA |
| | | GGTCTGGAAGCGGTTAATAAAGACAAACCGCTGGGTGCCGTAG |
| | | CGCTGAAGTCTTACGAGGAAGAGTTGGCGAAAGATCCACGTATT |
| | | GCCGCCACCATGGAAAACGCCCAGAAAGGTGAAATCATGCCGA |
| | | ACATCCCGCAGATGTCCGCTTTCTGGTATGCCGTGCGTACTGCG |
| | | GTGATCAACGCCGCCAGCGGTCGTCAGACTGTCGATGAAGCCCT |
| | | GAAAGACGCGCAGACTAATTCGAGCTCGAACAACAACAACAAT |
| | | AACAATAACAACAACCTCGGGATCGAGGGAAGGGGTACGCTCG |
| | | AGCTGGCGAGCTGCGTGCATCCGGATACCAAAGTGACCATTCGC |
| | | CGCAAACTGTGCAGCGAATTCGGATCCCCGCGTAAAGTGATTAA |
| | | AATGGAATCTGAAGAAAGATCTATGGACGAAAAAACCACCGGT |
| | | TGGCGTGGTGGTCACGTTGTTGAAGGTCTGGCTGGTGAACTGGA |
| | | ACAGCTGCGTGCTCGTCTGGAACACCACCCGCAGGGTCAGCGTG |
| | | AACCCTAA |
| 204 | Amino acid sequence | MGTSIEEKKVTVQELRELYLSGEYTIEIDTPDGYQTIGKWFDKGVLS |
| | (VidaL_UvsX-2)^{C}-Trx-His₆ | MVRVATATYETVCAFNHMIQLADNTWVQACELDVGVDIQTAAGI |
| | | QPVMLVEDTSDAECYDFEVMHPNHRYYGDGIVSHNSGKGSGGTG |
| | | MSDKIIHLTDDSFDTDVLKADGAILVDFWAEWCGPCKMIAPILDEI |
| | | ADEYQGKLTVAKLNIDQNPGTAPKYGIRGIPTLLLFKNGEVAATKV |
| | | GALSKGQLKEFLDANLAGSVDRSHHHHHH |
| 205 | DNA sequence of | ATGGGCACTAGTATTGAAGAAAAAAAAGTGACCGTGCAGGAAC |
| | (VidaL_UvsX-2)^{C}-Trx-His₆ | TGCGCGAACTGTATCTGAGCGGCGAATATACCATTGAAATTGAT |
| | encoding gene | ACCCCGGATGGCTATCAGACCATTGGCAAATGGTTTGATAAAGG |
| | | CGTGCTGAGCATGGTGCGCGTGGCGACCGCGACCTATGAAACCG |
| | | TGTGCGCGTTTAACCATATGATTCAGCTGGCGGATAACACCTGG |
| | | GTGCAGGCGTGCGAACTGGATGTGGGCGTGGATATTCAGACCGC |
| | | GGCGGGCATTCAGCCGGTGATGCTGGTGGAAGATACCAGCGAT |
| | | GCGGAATGCTATGATTTTGAAGTGATGCATCCGAACCATCGCTA |
| | | TTATGGCGATGGCATTGTGAGCCATAACAGCGGCAAAGGCAGC |
| | | GGCGGTACCGGTATGAGCGATAAAATTATTCACCTGACTGACGA |
| | | CAGTTTTGACACGGATGTACTCAAAGCGGACGGGGCGATCCTCG |
| | | TCGATTTCTGGGCAGAGTGGTGCGGTCCGTGCAAAATGATCGCC |
| | | CCGATTCTGGATGAGATCGCTGACGAATATCAGGGCAAACTGAC |
| | | CGTTGCAAAACTGAACATCGATCAAAACCCTGGCACTGCGCCGA |
| | | AATATGGCATCCGTGGTATCCCGACTCTGCTGCTGTTCAAAAAC |
| | | GGTGAAGTGGCGGCAACCAAAGTGGGTGCACTGTCTAAAGGTC |
| | | AGTTGAAAGAGTTCCTCGACGCTAACCTGGCCGGCTCTGTCGAC |
| | | AGATCTCATCACCATCACCATCACTAA |
| 206 | (VidaL_UvsX-2)^{N} peptide | ESGCLPKEAVVQIRLTKKGA |

1. Isolated polypeptide comprising at least one intein or at least one intein fragment of said intein, wherein said intein is a naturally split intein with a N-terminal intein fragment split after 14-60 amino acids from the intein's N-terminal position, wherein the polypeptide further comprises at least one heterologous C-terminal extein and/or at least one heterologous N-terminal extein sequence.

2. Isolated polypeptide of claim 1 wherein said at least one intein fragment, is selected from the group comprising:
a) a N-terminal intein fragment having 100% sequence identity with SEQ ID NO: 2, 3, 4, 5, 28, 38, 44, 50, 56, 62, 68, 74, 80, 86, 92, 98, 104, 110, 116, 122, 128, 134, 140, 146, 152, 158, 164, 170, 176 or 182, or,
b) a N-terminal intein fragment having at least 70%, or 80% or 85% or 90% or 95% sequence identity with SEQ ID NO: 2, 3, 4, 5, 28, 38, 44, 50, 56, 62, 68, 74, 80, 86, 92, 98, 104, 110, 116, 122, 128, 134, 140, 146, 152, 158, 164, 170, 176 or 182, and/or
c) a C-terminal intein fragment having 100% sequence identity with SEQ ID NO: 6, 27, 39, 45, 51, 57, 63, 69, 75, 81, 87, 93, 99, 105, 111, 117, 123, 129, 135, 141, 147, 153, 159, 165, 171, 177, 183, 194, 195 or 196 or,
d) a C-terminal intein fragment having at least 70%, or 80% or 85% or 90% or 95% sequence identity with SEQ ID NO: 6, 27, 39, 45, 51, 57, 63, 69, 75, 81, 87, 93, 99, 105, 111, 117, 123, 129, 135, 141, 147, 153, 159, 165, 171, 177, 183, 194, 195 or 196, or
e) wherein the at least one intein has at least 70%, or 80% or 85% or 90% or 95% sequence identity with SEQ ID NO:1 or 26.

3. Isolated polypeptide according to claim 1, wherein said polypeptide comprises at least one N-terminal intein fragment and at least one C-terminal intein fragment, wherein
1) if said at least one N-terminal intein fragment is selected from SEQ ID NO: 5 then said at least one C-terminal intein fragment is selected from SEQ ID NO: 6, or
2) if said at least one N-terminal intein fragment is selected from SEQ ID NO: 5, 28-33 then said at least one C-terminal intein fragment is selected from SEQ ID NO: 27, or
3) if said at least one N-terminal intein fragment is selected from SEQ ID NO: 38 then said at least one C-terminal intein fragment is selected from SEQ ID NO: 39, or
4) if said at least one N-terminal intein fragment is selected from SEQ ID NO: 44 then said at least one C-terminal intein fragment is selected from SEQ ID NO: 45, or
5) if said at least one N-terminal intein fragment is selected from SEQ ID NO: 50 then said at least one C-terminal intein fragment is selected from SEQ ID NO: 51, or
6) if said at least one N-terminal intein fragment is selected from SEQ ID NO: 56 then said at least one C-terminal intein fragment is selected from SEQ ID NO: 57, or
7) if said at least one N-terminal intein fragment is selected from SEQ ID NO: 62 then said at least one C-terminal intein fragment is selected from SEQ ID NO: 63, or
8) if said at least one N-terminal intein fragment is selected from SEQ ID NO: 68 then said at least one C-terminal intein fragment is selected from SEQ ID NO: 69, or
9) if said at least one N-terminal intein fragment is selected from SEQ ID NO: 74 then said at least one C-terminal intein fragment is selected from SEQ ID NO: 75, or
10) if said at least one N-terminal intein fragment is selected from SEQ ID NO: 80 then said at least one C-terminal intein fragment is selected from SEQ ID NO: 81, or
11) if said at least one N-terminal intein fragment is selected from SEQ ID NO: 86 then said at least one C-terminal intein fragment is selected from SEQ ID NO: 87, or
12) if said at least one N-terminal intein fragment is selected from SEQ ID NO: 92 then said at least one C-terminal intein fragment is selected from SEQ ID NO: 93, or
13) if said at least one N-terminal intein fragment is selected from SEQ ID NO: 98 then said at least one C-terminal intein fragment is selected from SEQ ID NO: 99, or
14) if said at least one N-terminal intein fragment is selected from SEQ ID NO: 104 then said at least one C-terminal intein fragment is selected from SEQ ID NO: 105, or
15) if said at least one N-terminal intein fragment is selected from SEQ ID NO: 110 then said at least one C-terminal intein fragment is selected from SEQ ID NO: 111, or
16) if said at least one N-terminal intein fragment is selected from SEQ ID NO: 116 then said at least one C-terminal intein fragment is selected from SEQ ID NO: 117, or
17) if said at least one N-terminal intein fragment is selected from SEQ ID NO: 122 then said at least one C-terminal intein fragment is selected from SEQ ID NO: 123, or
18) if said at least one N-terminal intein fragment is selected from SEQ ID NO: 128 then said at least one C-terminal intein fragment is selected from SEQ ID NO: 129, or
19) if said at least one N-terminal intein fragment is selected from SEQ ID NO: 134 then said at least one C-terminal intein fragment is selected from SEQ ID NO: 135, or
20) if said at least one N-terminal intein fragment is selected from SEQ ID NO: 140 then said at least one C-terminal intein fragment is selected from SEQ ID NO: 141, or
21) if said at least one N-terminal intein fragment is selected from SEQ ID NO: 146 then said at least one C-terminal intein fragment is selected from SEQ ID NO: 147, or
22) if said at least one N-terminal intein fragment is selected from SEQ ID NO: 152 then said at least one C-terminal intein fragment is selected from SEQ ID NO: 153, or
23) if said at least one N-terminal intein fragment is selected from SEQ ID NO: 158 then said at least one C-terminal intein fragment is selected from SEQ ID NO: 159, or
24) if said at least one N-terminal intein fragment is selected from SEQ ID NO: 164 then said at least one C-terminal intein fragment is selected from SEQ ID NO: 165, or
25) if said at least one N-terminal intein fragment is selected from SEQ ID NO: 170 then said at least one C-terminal intein fragment is selected from SEQ ID NO: 171, or
26) if said at least one N-terminal intein fragment is selected from SEQ ID NO: 176 then said at least one C-terminal intein fragment is selected from SEQ ID NO: 177, or
27) if said at least one N-terminal intein fragment is selected from SEQ ID NO: 182 then said at least one C-terminal intein fragment is selected from SEQ ID NO: 183, or
28) if said at least one N-terminal intein fragment is selected from SEQ ID NO: 2 then said at least one C-terminal intein fragment is selected from SEQ ID NO: 194, or
29) if said at least one N-terminal intein fragment is selected from SEQ ID NO: 3 then said at least one C-terminal intein fragment is selected from SEQ ID NO: 195, or
30) if said at least one N-terminal intein fragment is selected from SEQ ID NO: 3 then said at least one C-terminal intein fragment is selected from SEQ ID NO: 196.

4. Isolated polypeptide according to claim 1, wherein said polypeptide at the N-terminal end and/or at the C-terminal end of said at least one intein fragment further comprises a sequence selected from:
1) in the case of SEQ ID NO: 5 and/or 6; SEQ ID NO: 25 and/or 26, respectively,
2) in the case of SEQ ID NO: 5, 28-33 and/or 27 SEQ ID NO: 34 and/or 35, respectively,
3) in the case of SEQ ID NO:38 and/or 39; SEQ ID NO: 40 and/or 41, respectively,
4) in the case of SEQ ID NO:44 and/or 45; SEQ ID NO: 46 and/or 47, respectively,
5) in the case of SEQ ID NO:50 and/or 51; SEQ ID NO: 52 and/or 53, respectively,
6) in the case of SEQ ID NO:56 and/or 57; SEQ ID NO: 58 and/or 59, respectively,
7) in the case of SEQ ID NO:62 and/or 63; SEQ ID NO: 64 and/or 65, respectively,
8) in the case of SEQ ID NO:68 and/or 69; SEQ ID NO: 70 and/or 71, respectively,
9) in the case of SEQ ID NO:74 and/or 75; SEQ ID NO: 76 and/or 77, respectively,
10) in the case of SEQ ID NO:80 and/or 81; SEQ ID NO: 82 and/or 83, respectively,
11) in the case of SEQ ID NO:86 and/or 87; SEQ ID NO: 88 and/or 89, respectively,
12) in the case of SEQ ID NO:92 and/or 93; SEQ ID NO: 94 and/or 95, respectively,
13) in the case of SEQ ID NO: 98 and/or 99; SEQ ID NO: 100 and/or 101, respectively,
14) in the case of SEQ ID NO: 104 and/or 105; SEQ ID NO: 106 and/or 107, respectively,
15) in the case of SEQ ID NO: 110 and/or 111; SEQ ID NO: 112 and/or 113, respectively,
16) in the case of SEQ ID NO:116 and/or 117; SEQ ID NO: 118 and/or 119, respectively,
17) in the case of SEQ ID NO:122 and/or 123; SEQ ID NO: 124 and/or 125, respectively,
18) in the case of SEQ ID NO:128 and/or 129; SEQ ID NO: 130 and/or 131, respectively,
19) in the case of SEQ ID NO:134 and/or 135; SEQ ID NO: 136 and/or 137, respectively,
20) in the case of SEQ ID NO:140 and/or 141; SEQ ID NO: 142 and/or 143, respectively,
21) in the case of SEQ ID NO:146 and/or 147; SEQ ID NO: 148 and/or 149, respectively,
22) in the case of SEQ ID NO:152 and/or 153; SEQ ID NO: 154 and/or 155, respectively,
23) in the case of SEQ ID NO:158 and/or 159; SEQ ID NO: 160 and/or 161, respectively,
24) in the case of SEQ ID NO:164 and/or 165; SEQ ID NO: 166 and/or 167, respectively,
25) in the case of SEQ ID NO:170 and/or 171; SEQ ID NO: 172 and/or 173, respectively,
26) in the case of SEQ ID NO:176 and/or 177; SEQ ID NO: 178 and/or 179, respectively,
27) in the case of SEQ ID NO:182 and/or 183; SEQ ID NO: 184 and/or 185, respectively.

5. Isolated polypeptide according to claim 1, wherein the N-terminal intein fragment has 100% sequence identity with a sequence selected from sequences comprising SEQ ID NO: 5, 11 or 12 or has at least 90% or 95% sequence identity with a sequence selected from sequences comprising SEQ ID NO: 5, 11 or 12 and/or,
wherein the C-terminal intein fragment has 100% sequence identity with a sequence selected from sequences comprising SEQ ID NO:6 or 13-18 or has at least 90% or 95% sequence identity with a sequence selected from sequences comprising SEQ ID NO:6 or 13-18.

6. Isolated polypeptide according to claim 1, wherein the N-terminal intein fragment is selected from sequences comprising SEQ ID NO: 5, 11 or 12 and/or,
wherein the C-terminal intein fragment is selected from sequences comprising SEQ ID NO:6 or 13-18.

7. Isolated polypeptide according to claim 1, wherein the polypeptide comprises the N-terminal intein fragment with SEQ ID NO: 5 or 12 and the C-terminal intein fragment with SEQ ID NO: 13.

8. Isolated polypeptide according to claim 1, wherein the N-terminal intein fragment has 100% sequence identity with a sequence selected from sequences comprising SEQ ID NO: 5, 28-33 or has at least 90% or 95% sequence identity with a sequence selected from sequences comprising SEQ ID NOs: 28-33 and/or,
wherein the C-terminal intein fragment has 100% sequence identity with SEQ ID NO: 27 or has at least 90% or 95% sequence identity with SEQ ID NO: 27.

9. (canceled)

10. Two isolated polypeptides according to claim 1,
wherein one of the isolated polypeptides comprises least one heterologous N-terminal extein fused to a N-terminal intein fragment having at least 70%, or 80% or 85% or 90% or 95% or 99% sequence identity with SEQ ID NO: 5, 28, 38, 44, 50, 56, 62, 68, 74, 80, 86, 92, 98, 104, 110, 116, 122, 128, 134, 140, 146, 152, 158, 164, 170, 176 or 182 and,
wherein the second one of the isolated polypeptides comprises at least one C-terminal extein sequence fused to a C-terminal intein fragment having at least 70%, or 80% or 85% or 90% or 95% or 99% sequence identity with SEQ ID NO: 6, 27, 39, 45, 51, 57, 63, 69, 75, 81, 87, 93, 99, 105, 111, 117, 123, 129, 135, 141, 147, 153, 159, 165, 171, 177 or 183.

11. Isolated polypeptide according to claim 1, wherein the polypeptide further comprises at least one component selected from a solubility factor, a marker, a linker, an epitope, an affinity tag, a fluorophore or a fluorescent protein, a toxic compound or protein and a small-molecule or a small-molecule binding protein.

12. Isolated nucleic acid molecule comprising a nucleotide sequence encoding for an isolated polypeptide of any one of claims 1 to 8, 10-11 or a homolog, variant or complement thereof.

13-15. (canceled)
